(19) United States
(12) Patent Application Publication
(10) Pub. No.: US 2021/0179554 A1
(43) Pub. Date: Jun. 17, 2021

(54) ONIUM SALT COMPOUND, CHEMICALLY AMPLIFIED RESIST COMPOSITION AND PATTERNING PROCESS

(71) Applicant: Shin-Etsu Chemical Co., Ltd., Tokyo (JP)

(72) Inventors: Takayuki Fujiwara, Joetsu-shi (JP); Tomomi Watanabe, Joetsu-shi (JP); Kazuhiro Katayama, Joetsu-shi (JP)

(73) Assignee: Shin-Etsu Chemical Co., Ltd., Tokyo (JP)

(21) Appl. No.: 17/111,909

(22) Filed: Dec. 4, 2020

(30) Foreign Application Priority Data
Dec. 12, 2019 (JP) 2019-224690

(51) Int. Cl. C07C 381/12; C07D 327/08; G03F 7/32; G03F 7/004; G03F 7/038; C08L 33/14

(52) U.S. Cl. CPC C07C 381/12; C07D 327/08; C08L 33/14; G03F 7/0045; G03F 7/0382; G03F 7/325

(57) ABSTRACT

An onium salt having formula (1) serving as an acid diffusion inhibitor and a chemically amplified resist composition comprising the acid diffusion inhibitor are provided. When processed by lithography, the resist composition exhibits a high sensitivity, and excellent lithography performance factors such as CDU and LWR.

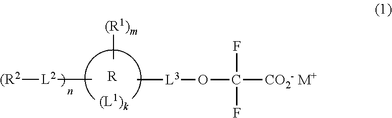

### CROSS-REFERENCE TO RELATED APPLICATION

This non-provisional application claims priority under 35 U.S.C. § 119(a) on Patent Application No. 2019-224690 filed in Japan on Dec. 12, 2019, the entire contents of which are hereby incorporated by reference.

### TECHNICAL FIELD

This invention relates to an onium salt compound, a chemically amplified resist composition, and a pattern forming process.

### BACKGROUND ART

To meet the demand for higher integration and operating speeds in LSIs, further miniaturization of the pattern rule is desired. The requirement to form resist patterns of high resolution necessitates not only to improve lithography properties as typified by pattern profile, contrast, mask error factor (MEF), depth of focus (DOF), critical dimension uniformity (CDU), and line width roughness (LWR), but also to minimize defects on the resist pattern after development.

As the pattern feature size is reduced, LWR becomes more noticeable. It is pointed out that LWR is affected by the segregation and agglomeration of a base polymer and an acid generator and acid diffusion. There is a propensity that LWR is degraded as the resist film becomes thinner. The degradation of LWR caused by resist film thinning to comply with further miniaturization becomes a serious problem.

For the EUV resist composition, it is necessary to achieve a high sensitivity, high resolution and low LWR at the same time. As the acid diffusion distance is shortened, the outcome is a smaller LWR, but a lower sensitivity. For example, when the PEB temperature is lowered, LWR becomes smaller, but sensitivity becomes lower. When the amount of an acid diffusion inhibitor or quencher added is increased, LWR becomes smaller, but sensitivity becomes lower. It is necessary to overcome the tradeoff relationship between sensitivity and LWR.

Studies have been made on various additives in order to overcome the tradeoff relationship between sensitivity and LWR. Means for enhancing sensitivity include the structural optimization of photoacid generators and acid diffusion inhibitors such as amines and weak acid onium salts and the addition of acid amplifiers. Patent Document 1 discloses an acid diffusion inhibitor of onium salt type having incorporated the mechanism that basicity is reduced by an acid. Yet a resist composition capable of meeting both sensitivity and LWR has not been developed.

As the acid diffusion inhibitor featuring improved LWR and other properties, Patent Documents 1 and 2 disclose onium salts containing anions having the following formulae.

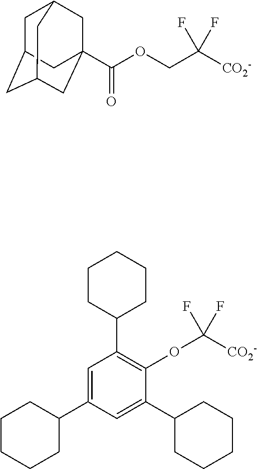

When these onium salts are used as an acid diffusion inhibitor, there are obtained no results satisfying various lithography factors for the current generation where ultrafine processing using ArF or EUV lithography is required.

### CITATION LIST

Patent Document 1: WO 2019/187445

Patent Document 2: JP 5904180 (U.S. Pat. No. 9,221,742)

### DISCLOSURE OF INVENTION

While resist patterns of high resolution are recently required, resist compositions comprising conventional acid diffusion inhibitors do not always meet lithography performance factors such as sensitivity, CDU, and LWR.

An object of the invention is to provide a chemically amplified resist composition which when processed by lithography using high-energy radiation such as KrF or ArF excimer laser, EB or EUV, exhibits a high sensitivity and is improved in lithography performance factors such as CDU and LWR. Another object is to provide an acid diffusion inhibitor used in the resist composition and a pattern forming process using the resist composition.

The inventors have found that a chemically amplified resist composition comprising an onium salt of carboxylic acid having a specific structure as an acid diffusion inhibitor exhibits a high sensitivity and improved lithography performance factors such as CDU and LWR, and is suited for high accuracy micropatterning.

In one aspect, the invention provides an onium salt compound having the formula (1).

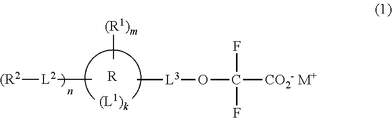

Herein m, n and k are each independently 0 or a positive integer, m+n+k is at least 1. R¹ is halogen, trifluoromethyl or trifluoromethoxy. R² is hydrogen or a C₁-C₁₅ hydrocarbyl group which may contain a heteroatom. L¹ is —C(═O)—, —C(═O)—O—, —S(═O)—, —S(═O)₂— or —S(═O)₂—O—. L² is *—C(═O)—, *—C(═O)—O—, *—S(═O)—, *—S(═O)₂— or *—S(═O)₂—O—, * designates a valence bond to the ring R. L³ is a single bond or C₁-C₁₅ hydrocarbylene group, some hydrogen in the hydrocarbylene group may be substituted by a heteroatom-containing moiety, —CH₂— in the hydrocarbylene group may be replaced by —O—, —C(═O)—, —S—, —S(═O)—, —S(═O)₂— or —N(R^{N})—, with the proviso that when L³ is a hydrocarbylene group, the carbon atom bonding to —OCF₂CO₂⁻ in the formula does not bond to a heteroatom other than the oxygen atom in the formula. R^{N} is hydrogen or a C₁-C₁₀ hydrocarbyl group, some hydrogen in the hydrocarbyl group R^{N} may be substituted by a heteroatom-containing moiety, —CH₂— in the hydrocarbyl group R^{N} may be replaced by —O—, —C(═O)—, or —S(═O)₂—. The ring R is a (m+n+1)-valent cyclic hydrocarbon group when k=0, and a (m+n+1)-valent cyclic hydrocarbon group containing k number of L¹ when k is a positive integer, some hydrogen in the cyclic hydrocarbon group may be substituted by a heteroatom-containing moiety, —CH₂— in the cyclic hydrocarbon group may be replaced by —O— or —S—. M⁺ is a sulfonium or iodonium cation.

In preferred embodiments, L³ is a single bond; the ring R is an aromatic hydrocarbon group; and m is an integer of at least 1.

In a preferred embodiment, the onium salt compound has the formula (2).

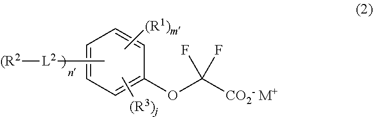

Herein R¹, R², L², and M⁺ are as defined above, m′ is an integer of 0 to 5, n′ is an integer of 0 to 5, and j is an integer of 0 to 4, 1≤m′+n′≤5 and 1≤m′+n′+j≤5. R³ is hydrogen, hydroxyl, carboxyl or a C₁-C₁₅ hydrocarbyl group, some hydrogen in the hydrocarbyl group may be substituted by a heteroatom-containing moiety, —CH₂— in the hydrocarbyl group may be replaced by —O— or —C(═O)—. When j is an integer of 2 to 4, a plurality of R³ may to be the same or different, or two R³ may bond together to form a ring with the carbon atoms to which they are attached.

In preferred embodiments, m′ is an integer of at least 1; and R¹ is iodine.

Also preferably, M⁺ is a cation having any one of the following formulae (M-1) to (M-4).

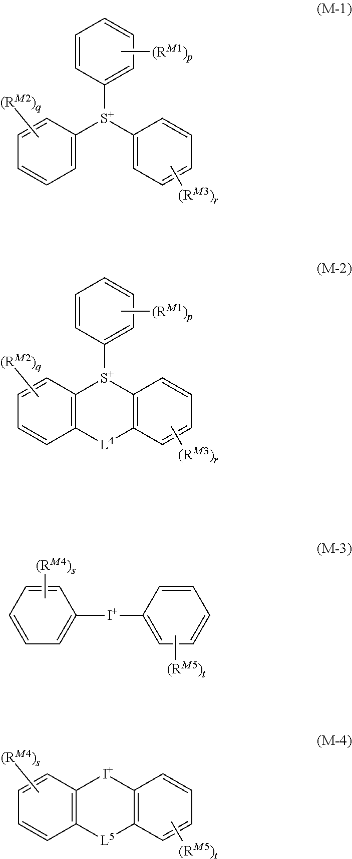

Herein R^{M1}, R^{M2}, R^{M3}, R^{M4}, and R^{M5} are each independently halogen, hydroxyl, or a C₁-C₁₅ hydrocarbyl group, some hydrogen in the hydrocarbyl group may be substituted by a heteroatom-containing moiety, —CH₂— in the hydrocarbyl group may be replaced by —O—, —C(═O)—, —S—, —S(═O)—, —S(═O)₂— or —N(R^{N})—. L⁴ and L⁵ are each independently a single bond, —CH₂—, —O—, —C(═O)—, —S—, —S(═O)—, —S(═O)₂— or —N(R^{N})—. R^{N} is hydrogen or a C₁-C₁₀ hydrocarbyl group, some hydrogen in the hydrocarbyl group may be substituted by a heteroatom-containing moiety, —CH₂— in the hydrocarbyl group may be replaced by —O—, —C(═O)— or —S(═O)₂—; p, q, r, s and t are each independently an integer of 0 to 5; when p is 2 or more, a plurality of R^{M1} may be the same or different, and two R^{M1} may bond together to form a ring with the carbon atoms on the benzene ring to which they are attached, when q is 2 or more, a plurality of R^{M2} may be the same or different, and two R^{M2} may bond together to form a ring with the carbon atoms on the benzene ring to which they are attached, when r is 2 or more, a plurality of R^{M3} may be the same or different, and two R^{M3} may bond together to form a ring with the carbon atoms on the benzene ring to which they are attached, when s is 2 or more, a plurality of R^{M4} may be the same or different, and two R^{M4} may bond together to form a ring with the carbon atoms on the benzene ring to which they are attached, when t is 2 or more, a plurality of R^{M5} may be the same or different, and two R^{M5} may bond together to form a ring with the carbon atoms on the benzene ring to which they are attached.

In a preferred embodiment, the onium salt compound has the following formula (3) or (4).

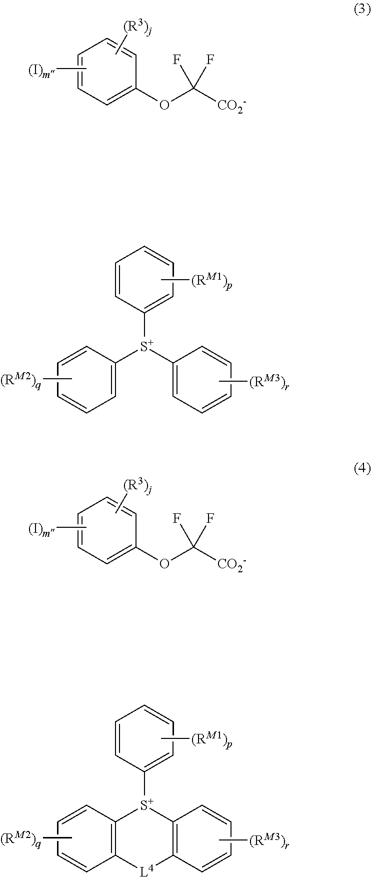

Herein R^{M1}, R^{M2}, R^{M3}, R³, L⁴, p, q, and r are as defined above, m is an integer of 1 to 5, and j is an integer of 0 to 4, and m″+j is from 1 to 5.

In another aspect, the invention provides an acid diffusion inhibitor comprising the onium salt compound defined above.

In a further aspect, the invention provides

a chemically amplified resist composition comprising (A) a base polymer adapted to change its solubility in a developer under the action of an acid, (B) a photoacid generator, (C) an acid diffusion inhibitor comprising the onium salt compound defined above, and (D) an organic solvent; or
a chemically amplified resist composition comprising (A′) a base polymer adapted to change its solubility in a developer under the action of an acid, the base polymer comprising recurring units having a function of generating an acid upon exposure to light, (C) an acid diffusion inhibitor comprising the onium salt compound defined above, and (D) an organic solvent.

In a preferred embodiment, the base polymer comprises recurring units having the formula (a) or recurring units having the formula (b).

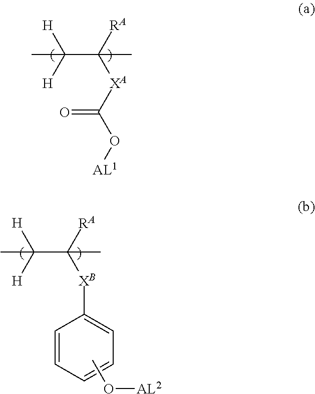

Herein R^{A} is hydrogen or methyl, X^{A} is a single bond, phenylene group, naphthylene group or (backbone)-C(═O)—O—X^{A1}—, X^{A1} is a C₁-C₁₅ hydrocarbylene group which may contain a hydroxyl moiety, ether bond, ester bond or lactone ring, X^{B} is a single bond or ester bond, AL¹ and AL² are each independently an acid labile group.

Preferably, the acid labile group has the formula (L1):

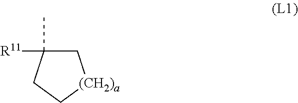

wherein R¹¹ is a C₁-C₇ hydrocarbyl group in which —CH₂— may be replaced by —O—, a is 1 or 2, and the broken line designates a valence bond.

In a preferred embodiment, the base polymer comprises recurring units having the formula (c):

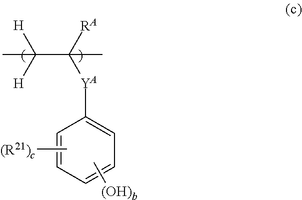

wherein R^{A} is hydrogen or methyl, Y^{A} is a single bond or ester bond, R²¹ is fluorine, iodine or a C₁-C₁₀ hydrocarbyl group in which —CH₂— may be replaced by —O— or —C(═O)—, b is an integer of 1 to 5, c is an integer of 0 to 4, and b+c is from 1 to 5.

Preferably, the recurring units having a function of generating an acid upon exposure to light are units of at least one type selected from the formulae (d1) to (d4).

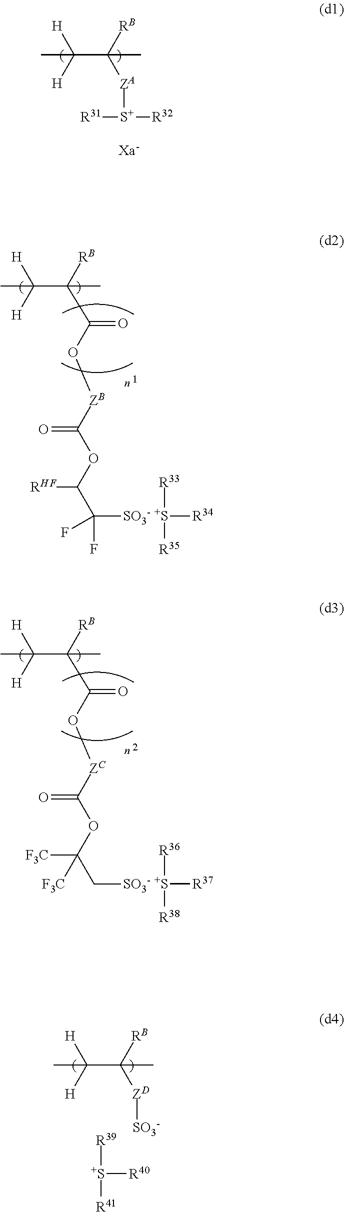

Herein R^{B} is hydrogen, fluorine, methyl or trifluoromethyl. Z^{A} is a single bond, phenylene group, —O—Z^{A1}—, —C(═O)—O—Z^{A1}— or —C(═O)—NH—Z^{A1}—, Z^{A1} is a C₁-C₂₀ hydrocarbylene group which may contain a heteroatom. Z^{B} and Z^{C} are each independently a single bond or a C₁-C₂₀ hydrocarbylene group which may contain a heteroatom. Z^{D} is a single bond, methylene, ethylene, phenylene, fluorinated phenylene, —O—Z^{D1}—, —C(═O)—O—Z^{D1}— or —C(═O)—NH—Z^{D1}—, Z^{D1} is an optionally substituted phenylene group. R³¹ to R⁴¹ are each independently a C₁-C₂₀ hydrocarbyl group which may contain a heteroatom, any two of Z^{A}, R³¹ and R³² may bond together to form a ring with the sulfur atom to which they are attached, any two of R³³, R³⁴ and R³⁵, any two of R³⁶, R³⁷ and R³⁸, and any two of R³⁹, R⁴⁰ and R⁴¹ may bond together to form a ring with the sulfur atom to which they are attached. R^{HF} is hydrogen or trifluoromethyl, n¹ is 0 or 1, n¹ is 0 when Z^{B} is a single bond, n² is 0 or 1, n² is 0 when Z^{C} is a single bond. Xa⁻ is a non-nucleophilic counter ion.

In a still further aspect, the invention provides a pattern forming process comprising the steps of applying the chemically amplified resist composition defined above to form a resist film on a substrate, exposing a selected region of the resist film to KrF excimer laser, ArF excimer laser, EB or EUV, and developing the exposed resist film in a developer.

In one preferred embodiment, the developing step uses an alkaline aqueous solution as the developer, thereby forming a positive pattern in which an exposed region of the resist film is dissolved away and an unexposed region of the resist film is not dissolved.

In another preferred embodiment, the developing step uses an organic solvent as the developer, thereby forming a negative pattern in which an unexposed region of the resist film is dissolved away and an exposed region of the resist film is not dissolved.

Typically, the organic solvent is at least one solvent selected from the group consisting of 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate.

### Advantageous Effects of Invention

The inventive chemically amplified resist composition comprising the onium salt compound as an acid diffusion inhibitor has a high sensitivity. When the resist composition is processed by lithography, a resist pattern having improved lithography performance factors such as CDU and LWR can be formed.

### DESCRIPTION OF THE PREFERRED EMBODIMENTS

The singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. The terms “group” and “moiety” are interchangeable. In chemical formulae, the broken line denotes a valence bond; Me stands for methyl, tBu for tert-butyl, Ac for acetyl, and Ph for phenyl. It is understood that for some structures represented by chemical formulae, there can exist enantiomers and diastereomers because of the presence of asymmetric carbon atoms. In such a case, a single formula collectively represents all such isomers. The isomers may be used alone or in admixture.

The abbreviations have the following meaning.

EB: electron beam
EUV: extreme ultraviolet
GPC: gel permeation chromatography
Mw: weight average molecular weight
Mw/Mn: molecular weight dispersity
PAG: photoacid generator
PEB: post-exposure bake
LWR: line width roughness
CDU: critical dimension uniformity

### Onium Salt

The invention provides an onium salt compound having the formula (1).

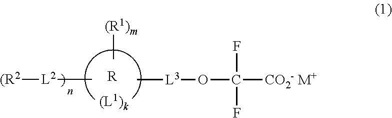

In formula (1), m, n and k are each independently 0 or a positive integer, m+n+k is at least 1. Preferably, m+n is at least 1, and more preferably m is an integer of at least 1. Also preferably m is an integer of 0 to 4, n is an integer of 0 to 4, k is an integer of 0 to 3, and m+n+k is from 1 to 5.

In formula (1), R¹ is halogen, trifluoromethyl or trifluoromethoxy, preferably fluorine, iodine, trifluoromethyl or trifluoromethoxy, more preferably iodine.

In formula (1), R² is hydrogen or a C₁-C₁₅ hydrocarbyl group which may contain a heteroatom. The C₁-C₁₅ hydrocarbyl group may be saturated or unsaturated and straight, branched or cyclic. Examples include alkyl groups such as methyl, ethyl, propyl, isopropyl, n-butyl, sec-butyl, tert-butyl, n-pentyl, n-hexyl, n-octyl, n-nonyl, n-decyl, 2-ethylhexyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, adamantylmethyl; alkenyl groups such as vinyl, allyl, propenyl, butenyl, hexenyl; unsaturated alicyclic hydrocarbyl groups such as cyclohexenyl; aryl groups such as phenyl, naphthyl, thienyl, 4-hydroxyphenyl, 4-methoxyphenyl, 3-methoxyphenyl, 2-methoxyphenyl, 4-ethoxyphenyl, 4-tert-butoxyphenyl, 3-tert-butoxyphenyl, 2-methylphenyl, 3-methylphenyl, 4-methylphenyl, 4-ethylphenyl, 4-tert-butylphenyl, 4-n-butylphenyl, 2,4-dimethylphenyl, 2,4,6-triisopropylphenyl, methylnaphthyl, ethylnaphthyl, methoxynaphthyl, ethoxynaphthyl, n-propoxynaphthyl, n-butoxynaphthyl, dimethylnaphthyl, diethylnaphthyl, dimethoxynaphthyl, diethoxynaphthyl; aralkyl groups such as benzyl, 1-phenylethyl, 2-phenylethyl, and combinations thereof. In the hydrocarbyl group, some hydrogen may be substituted by a moiety containing a heteroatom such as oxygen, sulfur, nitrogen or halogen, and a moiety containing a heteroatom such as oxygen, sulfur or nitrogen may intervene in a carbon-carbon bond, so that the group may contain a hydroxyl moiety, cyano moiety, carbonyl moiety, ether bond, ester bond, sulfonate bond, carbonate bond, lactone ring, sultone ring, carboxylic anhydride or haloalkyl moiety.

In formula (1), L¹ is —C(═O)—, —C(═O)—O—, —S(═O)—, —S(═O)₂— or —S(═O)₂—O—, preferably —C(═O)— or —C(═O)—O—.

In formula (1), L² is *—C(═O)—, *—C(═O)—O—, *—S(═O)—, *—S(═O)₂— or *—S(═O)₂—O—, preferably, *—C(═O)— or *—C(═O)—O—, wherein asterisk (*) designates a valence bond to the ring R.

In formula (1), L³ is a single bond or C₁-C₁₅ hydrocarbylene group. Some hydrogen in the hydrocarbylene group may be substituted by a heteroatom-containing moiety, and —CH₂— in the hydrocarbylene group may be replaced by —O—, —C(═O)—, —S—, —S(═O)—, —S(═O)₂— or —N(R^{N})—. R^{N} is hydrogen or a C₁-C₁₀ hydrocarbyl group, some hydrogen in the hydrocarbyl group R^{N} may be substituted by a heteroatom-containing moiety, and —CH₂— in the hydrocarbyl group R^{N} may be replaced by —O—, —C(═O)—, or —S(═O)₂—. It is noted that the constituent —CH₂— in the hydrocarbyl group may be one bonding to the ring R in formula (1). It is provided that when L³ is a hydrocarbylene group, the carbon atom bonding to —OCF₂CO₂⁻ in the formula does not bond to a heteroatom other than the oxygen atom in the formula. This means that in the following formula, the atoms (R*¹, R*² and R*³) to which C* bonds are hydrogen or carbon.

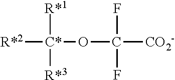

The hydrocarbylene group L³ may be saturated or unsaturated and straight, branched or cyclic. Examples thereof include alkanediyl groups such as methylene, ethylene, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, and tetradecane-1,14-diyl; cyclic saturated hydrocarbylene groups such as cyclopentanediyl, cyclohexanediyl, norbornanediyl, and adamantanediyl; arylene groups such as phenylene, methylphenylene, ethylphenylene, n-propylphenylene, isopropylphenylene, n-butylphenylene, isobutylphenylene, sec-butylphenylene, tert-butylphenylene, dimethylphenylene, diethylphenylene, naphthylene, methylnaphthylene, ethylnaphthylene, n-propylnaphthylene, isopropylnaphthylene, n-butylnaphthylene, isobutylnaphthylene, sec-butylnaphthylene, tert-butylnaphthylene, dimethylnaphthylene, diethylnaphthylene, and combinations thereof. Some hydrogen in the hydrocarbylene group may be substituted by a moiety containing a heteroatom such as oxygen, sulfur, nitrogen or halogen, and —CH₂— in the hydrocarbylene group may be replaced by —O—, —C(═O)—, —S—, —S(═O)—, —S(═O)₂— or —N(R^{N})—, so that the group may contain a hydroxyl moiety, cyano moiety, carbonyl moiety, ether bond, ester bond, amide bond, thioether bond, sulfinyl moiety, sulfonyl moiety, sulfonate bond, carbonate bond, lactone ring, sultone ring, carboxylic anhydride or haloalkyl moiety. R^{N} is as defined and exemplified above.

Preferably L³ is a single bond.

Examples of the group R²-L²- are given below, but not limited thereto.

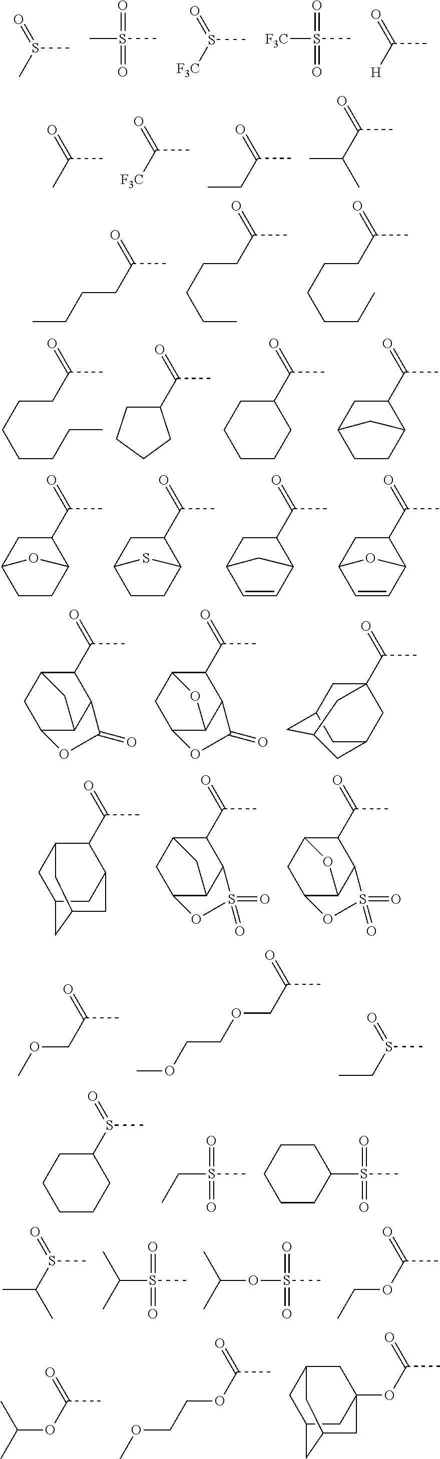

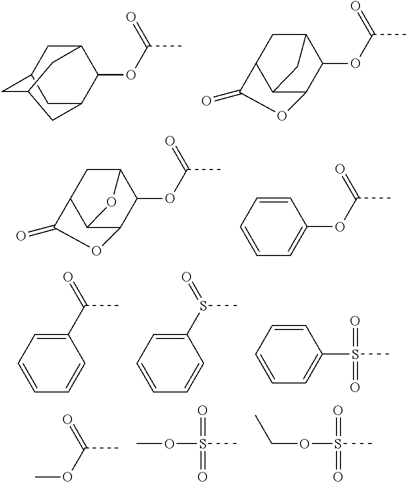

Herein the broken line designates a valence bond to the ring R.

In formula (1), the ring R is a (m+n+1)-valent cyclic hydrocarbon group when k=0, and a (m+n+1)-valent cyclic hydrocarbon group containing k number of L¹ when k is a positive integer. That is, the cyclic hydrocarbon group is obtained by removing (m+n+1) number of hydrogen atoms on the ring from a cyclic hydrocarbon or a cyclic hydrocarbon containing k number of L¹.

The cyclic hydrocarbon may be a compound composed solely of a ring or rings, or a compound composed of a ring or rings in which some or all of the hydrogen atoms on the ring(s) are substituted by hydrocarbyl moieties. The number of carbon atoms to form the ring(s) is preferably 3 to 15. The hydrocarbyl group preferably has 1 to 15 carbon atoms. Where a plurality of hydrocarbyl groups are contained, they may be the same or different and may bond together to form a ring with the carbon atoms to which they are attached. Further, some or all of the hydrogen atoms in the ring and/or hydrocarbyl group may be substituted by a moiety containing a heteroatom such as oxygen, sulfur, nitrogen or halogen, and —CH₂— in the ring and/or hydrocarbyl group may be replaced by —O— or —C(═O)—.

Examples of the cyclic hydrocarbon providing ring R wherein k=0 are shown below, but not limited thereto.

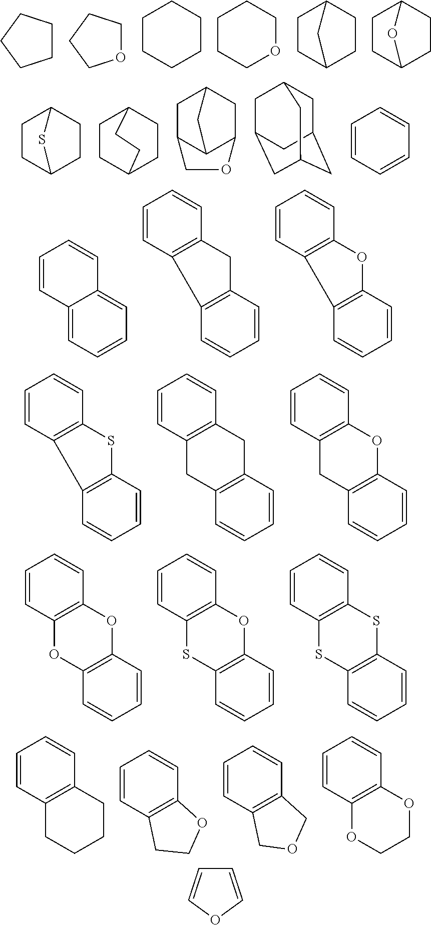

Examples of the cyclic hydrocarbon providing ring R wherein k≥1 are shown below, but not limited thereto.

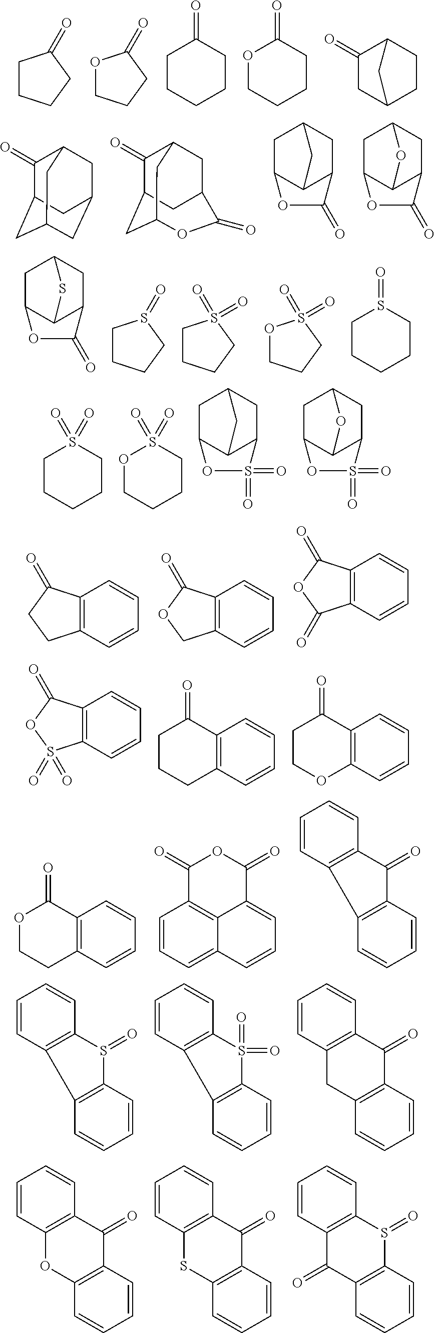

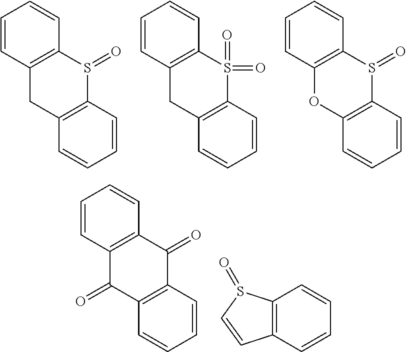

The ring R is preferably selected from benzene, adamantane, fluorene, and 1,9-dihydroanthracene rings, the foregoing rings in which —CH₂— is replaced by —C(═O)—, —S—, —S(═O)— or —S(═O)₂—, and the foregoing rings containing a norbornane lactone ring, more preferably the foregoing rings containing an aromatic ring, even more preferably benzene ring.

Of the onium salt compounds having formula (1), those compounds having the formula (2) are preferred.

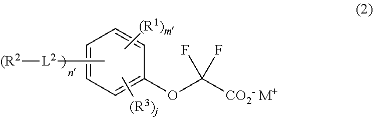

In formula (2), R¹, R², L², and M⁺ are as defined above.

In formula (2), R³ is hydrogen, hydroxyl, carboxyl or a C₁-C₁₅ hydrocarbyl group. The hydrocarbyl group may be saturated or unsaturated and straight, branched or cyclic. Examples include alkyl groups such as methyl, ethyl, propyl, isopropyl, n-butyl, sec-butyl, tert-butyl, n-pentyl, n-hexyl, n-octyl, 2-ethylhexyl, n-nonyl, n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, adamantylmethyl; alkenyl groups such as vinyl, allyl, propenyl, butenyl, hexenyl; unsaturated alicyclic hydrocarbyl groups such as cyclohexenyl; aryl groups such as phenyl, naphthyl, thienyl, 4-hydroxyphenyl, 4-methoxyphenyl, 3-methoxyphenyl, 2-methoxyphenyl, 4-ethoxyphenyl, 4-tert-butoxyphenyl, 3-tert-butoxyphenyl, 2-methylphenyl, 3-methylphenyl, 4-methylphenyl, 4-ethylphenyl, 4-tert-butylphenyl, 4-n-butylphenyl, 2,4-dimethylphenyl, 2,4,6-triisopropylphenyl, methylnaphthyl, ethylnaphthyl, methoxynaphthyl, ethoxynaphthyl, n-propoxynaphthyl, n-butoxynaphthyl, dimethylnaphthyl, diethylnaphthyl, dimethoxynaphthyl, diethoxynaphthyl; aralkyl groups such as benzyl, 1-phenylethyl, 2-phenylethyl, and combinations thereof.

Some or all of the hydrogen atoms in the hydrocarbyl group may be substituted by a moiety containing a heteroatom such as oxygen, sulfur, nitrogen or halogen, and —CH₂— in the hydrocarbyl group may be replaced by —O— or —C(═O)—, so that the group may contain a hydroxyl moiety, cyano moiety, carbonyl moiety, ether bond, ester bond, carbonate bond, lactone ring, carboxylic anhydride or haloalkyl moiety. It is noted that the constituent —CH₂— in the hydrocarbyl group may be one bonding to a carbon atom on the benzene ring in formula (2). In this case, R³ may be a hydrocarbyloxy, hydrocarbylcarbonyl, hydrocarbylcarbonyloxy, or hydrocarbyloxycarbonyl group.

Of these, R³ is preferably selected from hydrogen, alkyl groups such as methyl and tert-butyl, hydroxyl, carboxyl, alkoxy groups such as methoxy, 2-methoxyethoxy, and tert-butoxy, alkoxyalkoxy groups such as methoxymethoxy, alkoxycarbonyloxy groups such as tert-butoxycarbonyloxy, alkylcarbonyloxy groups such as acetoxy and trifluoroacetoxy, and alkoxycarbonyl groups such as tert-butoxycarbonyl; more preferably from hydrogen, hydroxyl, carboxyl, alkoxy, alkoxycarbonyl, and alkylcarbonyloxy groups.

In formula (2), m′ is an integer of 0 to 5, n′ is an integer of 0 to 5, and j is an integer of 0 to 4, 1≤m′+n′≤5 and 1≤m′+n′+j≤5; preferably m′ is an integer of 0 to 3, n′ is an integer of 0 to 2, and j is an integer of 0 to 4, 1≤m′+n′≤4 and 1≤m′+n′+j≤5; 113 more preferably m′ is an integer of 1 to 3, n′ is an integer of 0 to 2, and j is an integer of 0 to 4, 1≤m′+n′≤4 and 1≤m′+n′+j≤5.

When j is an integer of 2 to 4, a plurality of R³ may be the same or different, or two R³ may bond together to form a ring with the carbon atoms to which they are attached. Examples of the ring are shown below, but not limited thereto.

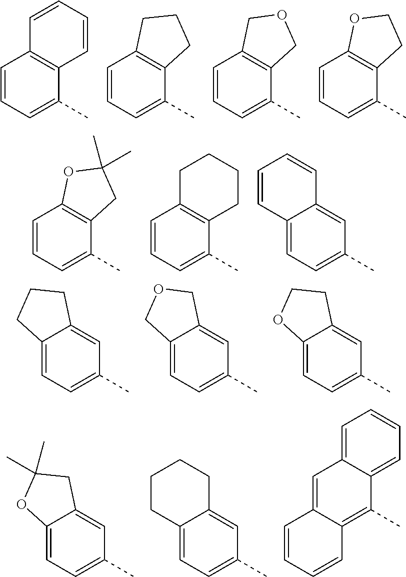

Herein the broken line designates a point of attachment to —OCF₂CO₂⁻.

In formulae (1) and (2), M⁺ is a sulfonium or iodonium cation, preferably containing at least one aromatic ring.

The preferred sulfonium or iodonium cation is selected from the following formulae (M-1) to (M-4).

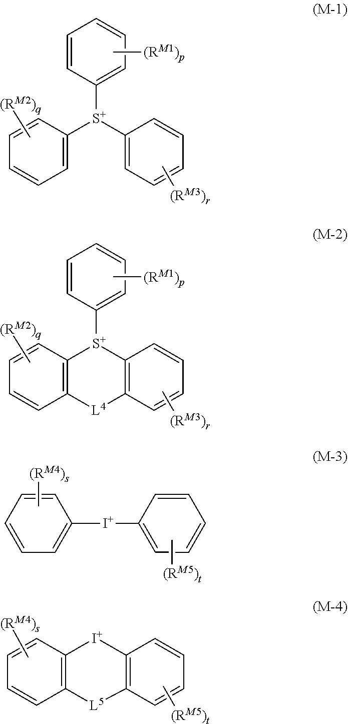

In formulae (M-1) to (M-4), R^{M1}, R^{M2}, R^{M3}, R^{M4}, and RMS are each independently halogen, hydroxyl, or a C₁-C₁₅ hydrocarbyl group. Suitable halogen atoms include fluorine, chlorine, bromine and iodine. The C₁-C₁₅ hydrocarbyl group may be saturated or unsaturated and straight, branched or cyclic. Examples include alkyl groups such as methyl, ethyl, propyl, isopropyl, n-butyl, sec-butyl, tert-butyl, n-pentyl, n-hexyl, n-octyl, 2-ethylhexyl, n-nonyl, n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, adamantylmethyl; alkenyl groups such as vinyl, allyl, propenyl, butenyl, hexenyl; unsaturated alicyclic hydrocarbyl groups such as cyclohexenyl; aryl groups such as phenyl, naphthyl, thienyl, 4-hydroxyphenyl, 4-methoxyphenyl, 3-methoxyphenyl, 2-methoxyphenyl, 4-ethoxyphenyl, 4-tert-butoxyphenyl, 3-tert-butoxyphenyl, 2-methylphenyl, 3-methylphenyl, 4-methylphenyl, 4-ethylphenyl, 4-tert-butylphenyl, 4-n-butylphenyl, 2,4-dimethylphenyl, 2,4,6-triisopropylphenyl, methylnaphthyl, ethylnaphthyl, methoxynaphthyl, ethoxynaphthyl, n-propoxynaphthyl, n-butoxynaphthyl, dimethylnaphthyl, diethylnaphthyl, dimethoxynaphthyl, diethoxynaphthyl; aralkyl groups such as benzyl, 1-phenylethyl, 2-phenylethyl, and combinations thereof.

Some or all of the hydrogen atoms in the hydrocarbyl group may be substituted by a moiety containing a heteroatom such as oxygen, sulfur, nitrogen or halogen, so that the group contains a hydroxyl, cyano, or haloalkyl moiety. Also, —CH₂— in the hydrocarbyl group may be replaced by —O—, —C(═O)—, —S—, —S(═O)—, —S(═O)₂— or —N(R^{N})— wherein R^{N} is as defined above. The constituent —CH₂— in the hydrocarbyl group may be one bonding to a carbon atom on the benzene ring in formulae (M-1) to (M-4). In this case, R^{M1} to RMS may be hydrocarbyloxy, hydrocarbylcarbonyloxy, hydrocarbylthio, hydrocarbylcarbonyl, hydrocarbylsulfonyl, or hydrocarbylamino.

In formulae (M-2) and (M-4), L⁴ and L⁵ are each independently a single bond, —CH₂—, —O—, —C(═O)—, —S—, —S(═O)—, —S(═O)₂— or —N(R^{N})—, wherein R^{N} is as defined above.

In formulae (M-1) to (M-4), p, q, r, s and t are each independently an integer of 0 to 5. When p is 2 or more, a plurality of R^{M1} may be the same or different, and two R^{M1} may bond together to form a ring with the carbon atoms on the benzene ring to which they are attached. When q is 2 or more, a plurality of R^{M2} may be the same or different, and two R^{M2} may bond together to form a ring with the carbon atoms on the benzene ring to which they are attached. When r is 2 or more, a plurality of R^{M3} may be the same or different, and two R^{M3} may bond together to form a ring with the carbon atoms on the benzene ring to which they are attached. When s is 2 or more, a plurality of R^{M4} may be the same or different, and two R^{M4} may bond together to form a ring with the carbon atoms on the benzene ring to which they are attached. When t is 2 or more, a plurality of R^{M5} may be the same or different, and two R^{M5} may bond together to form a ring with the carbon atoms on the benzene ring to which they are attached.

Examples of the sulfonium cation having formula (M-1) are given below, but not limited thereto.

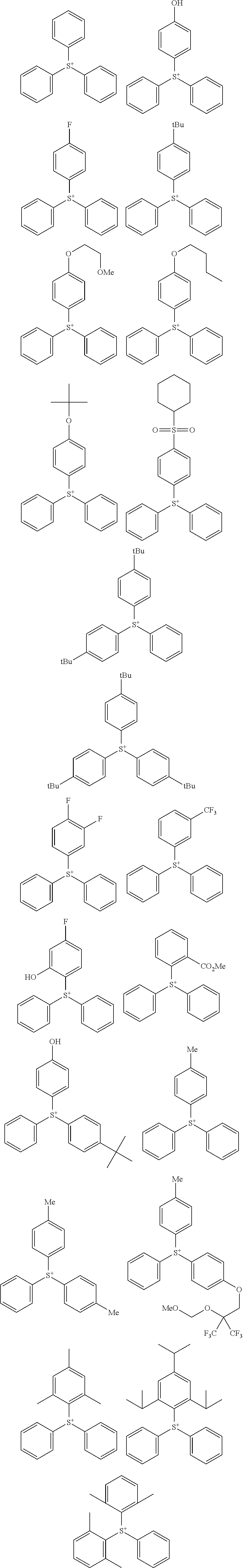

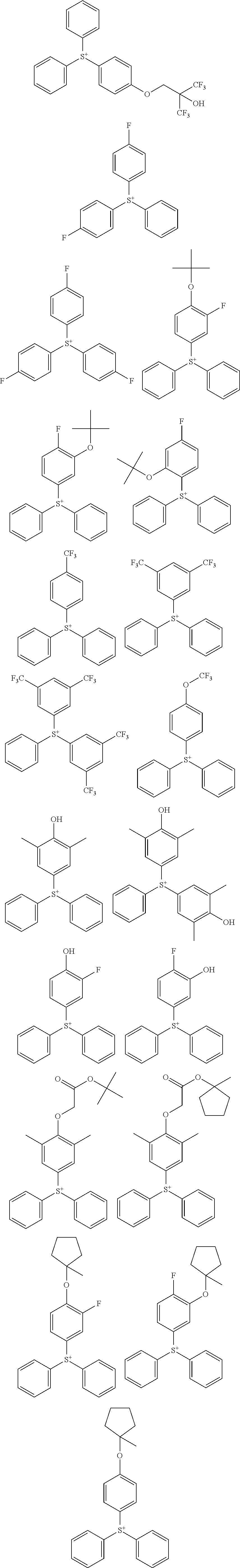

Examples of the sulfonium cation having formula (M-2) are given below, but not limited thereto.

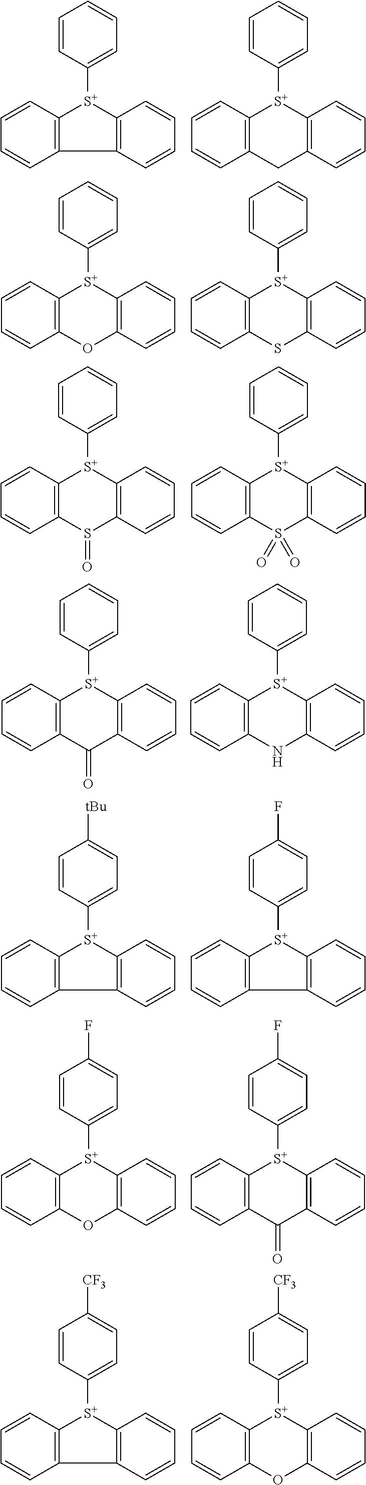

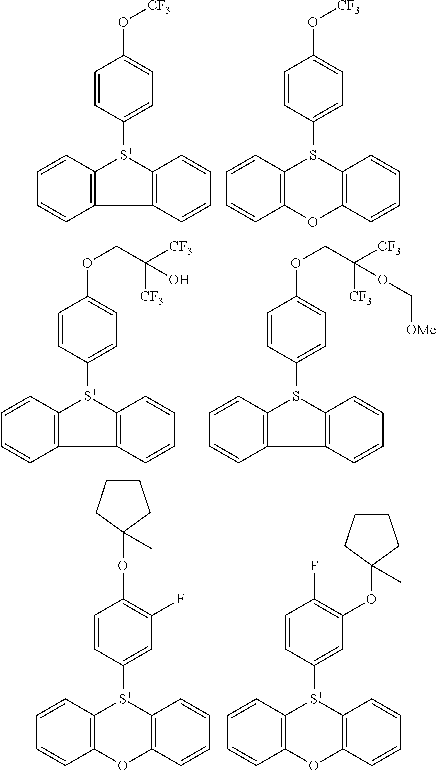 [SMILES: FC(F)(F)Oc1ccc(-[s+]2c3ccccc3c3ccccc32)cc1 ; FC(F)(F)Oc1ccc([S+]2c3ccccc3Oc3ccccc32)cc1 ; OC(COc1ccc(-[s+]2c3ccccc3c3ccccc32)cc1)(C(F)(F)F)C(F)(F)F ; COCOC(COc1ccc(-[s+]2c3ccccc3c3ccccc32)cc1)(C(F)(F)F)C(F)(F)F ; CC1(Oc2ccc([S+]3c4ccccc4Oc4ccccc43)cc2F)CCCC1 ; CC1(Oc2cc([S+]3c4ccccc4Oc4ccccc43)ccc2F)CCCC1]

Examples of the iodonium cation having formula (M-3) are given below, but not limited thereto.

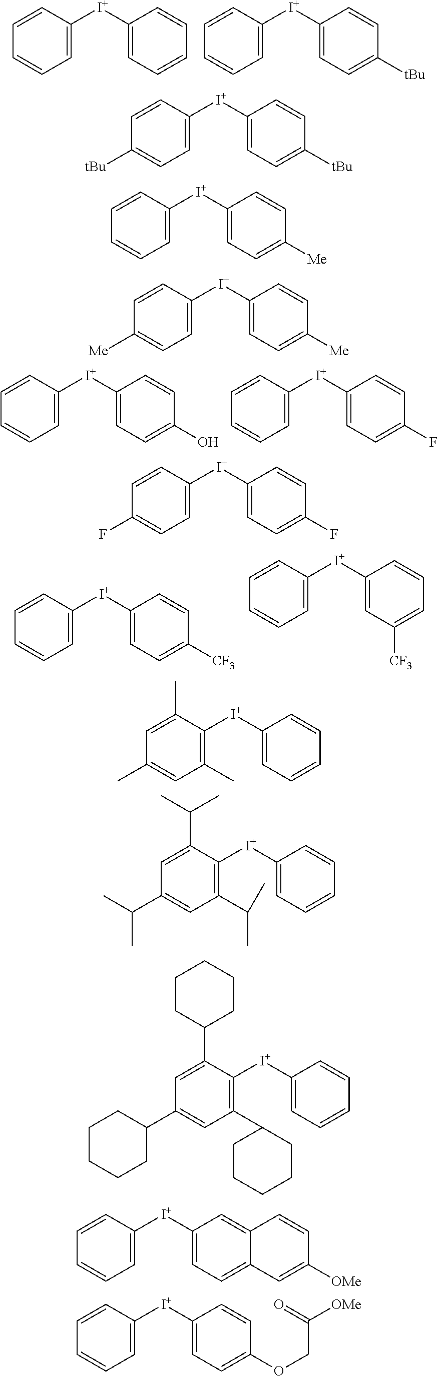

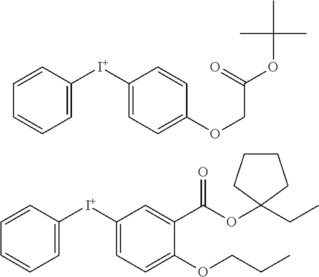 [SMILES: CC(C)(C)OC(=O)COc1ccc([I+]c2ccccc2)cc1 ; CCCOc1ccc([I+]c2ccccc2)cc1C(=O)OC1(CC)CCCC1]

Examples of the iodonium cation having formula (M-4) are given below, but not limited thereto.

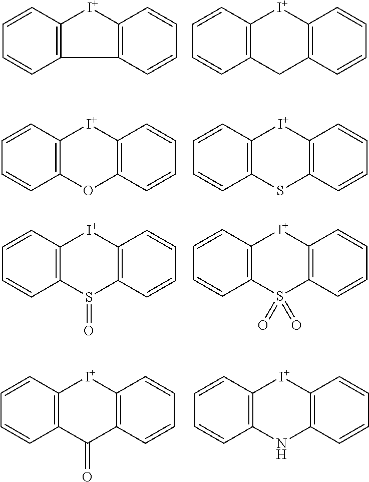

Suitable sulfonium cations other than the sulfonium cations having formulae (M-1) and (M-2) are given below, but not limited thereto.

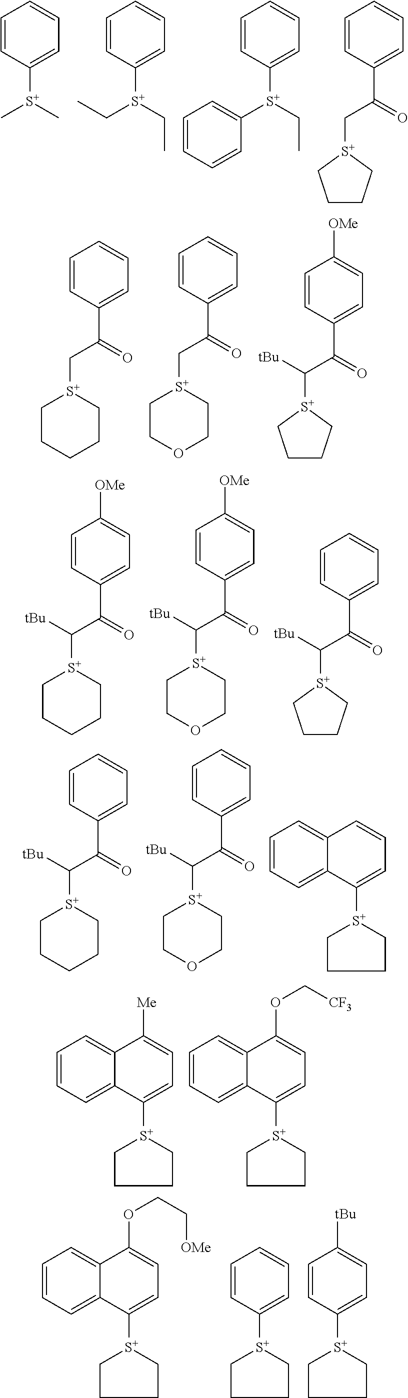

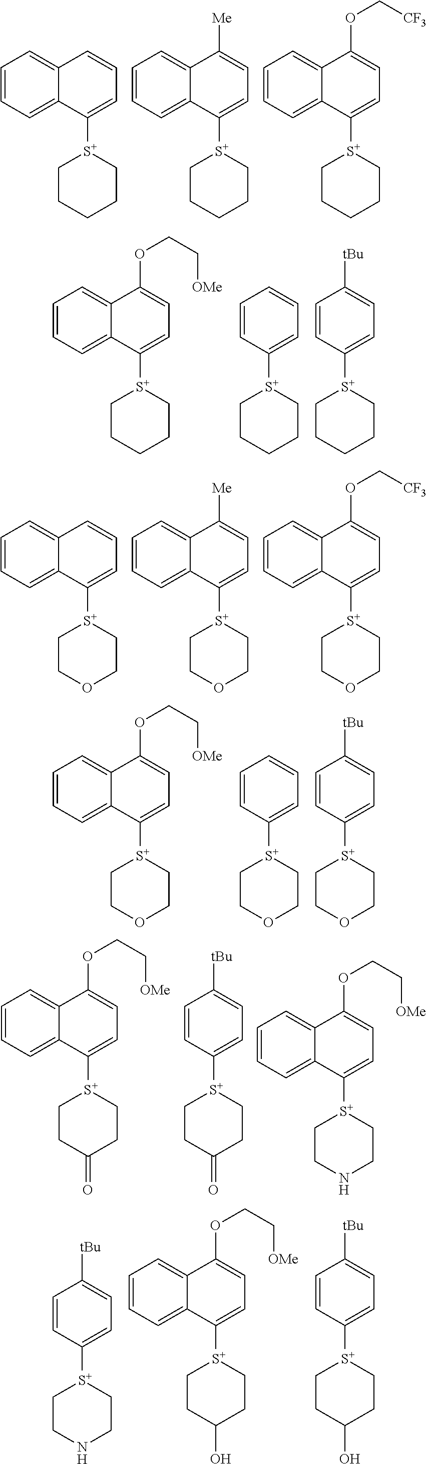

Of the compounds having formula (2), compounds having the following formulae (3) and (4) are more preferred.

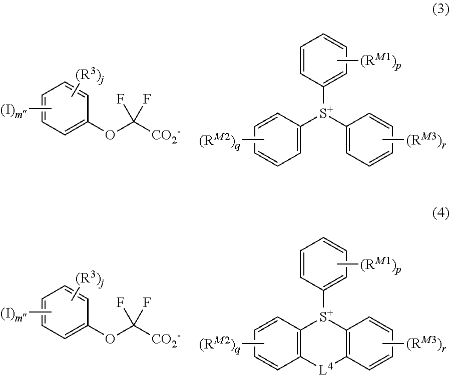

Herein R^{M1}, R^{M2}, R^{M3}, R³, L⁴, p, q, and r are as defined above; m″ is an integer of 1 to 5, j is an integer of 0 to 4, and m″+j is from 1 to 5.

Examples of the anion in the onium salt compound having formula (1) are shown below, but not limited thereto.

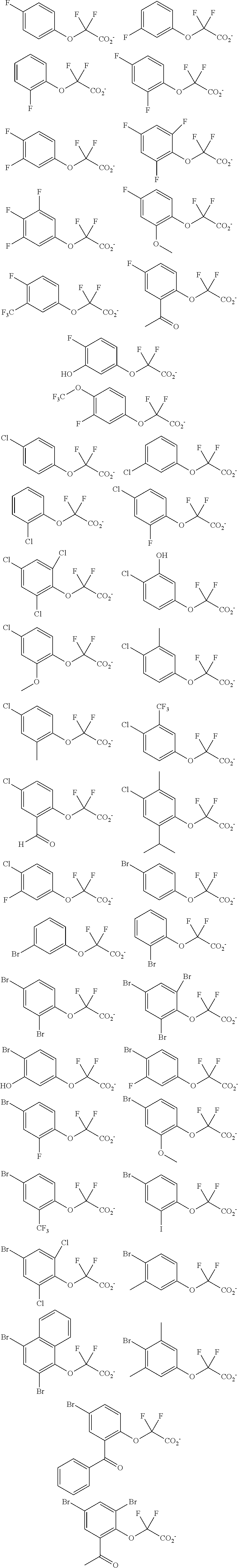

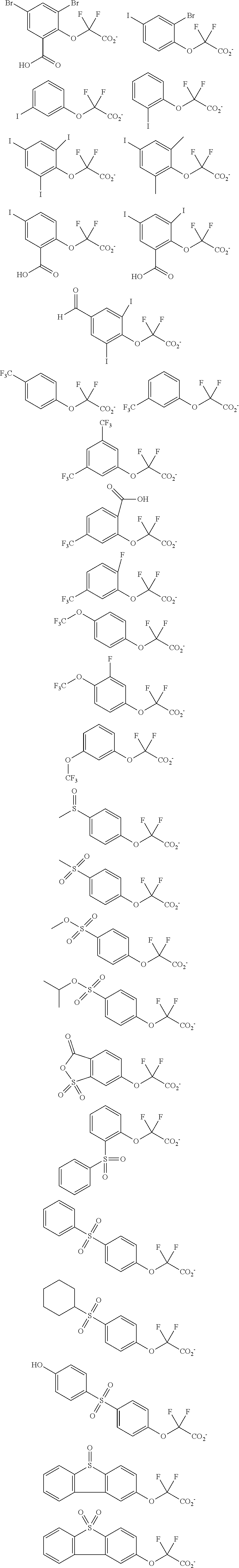

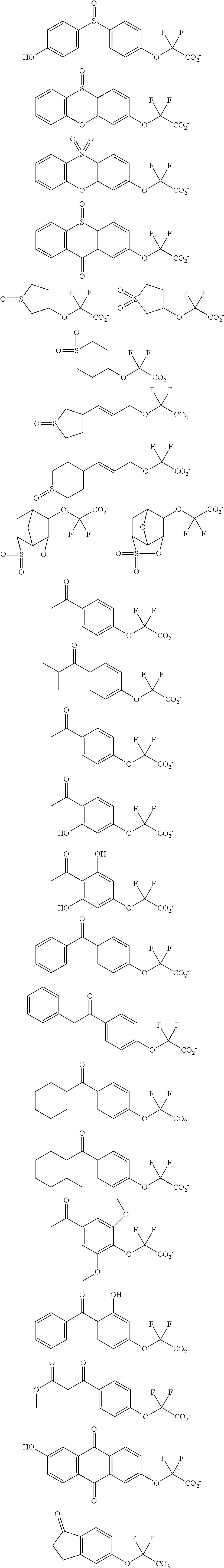

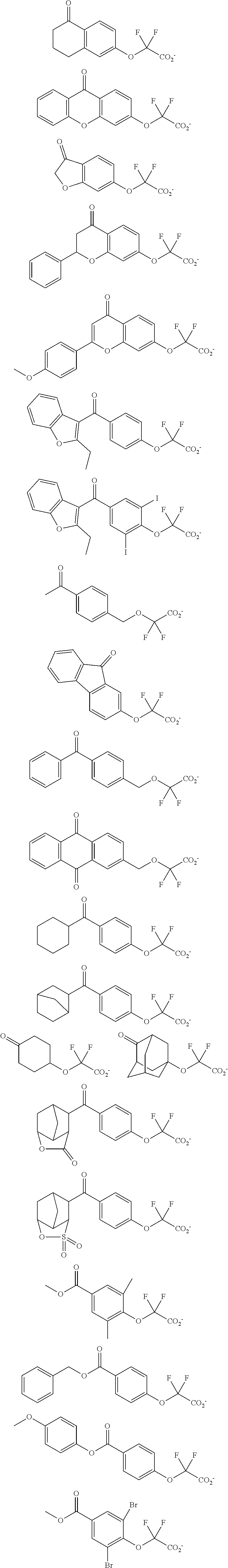

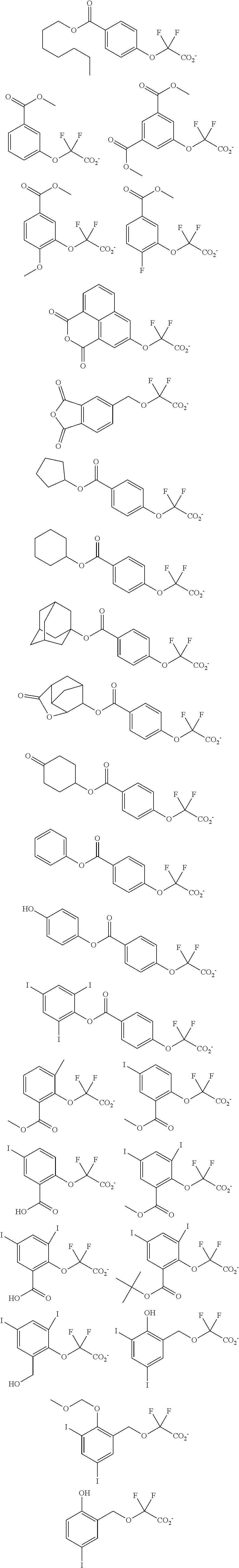

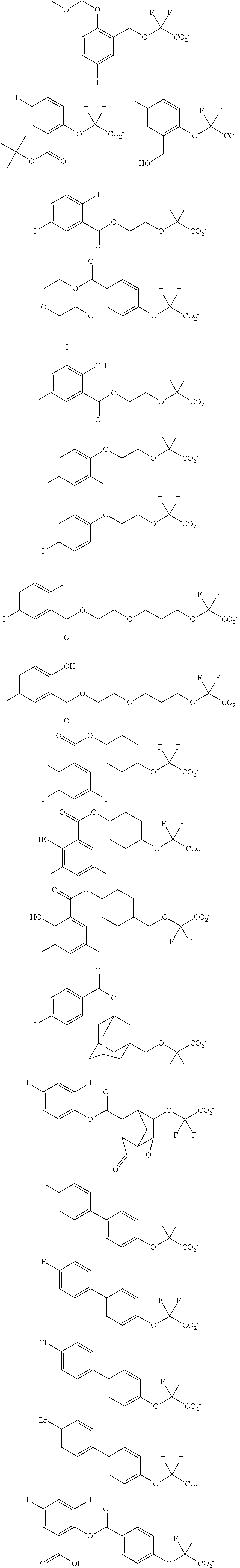

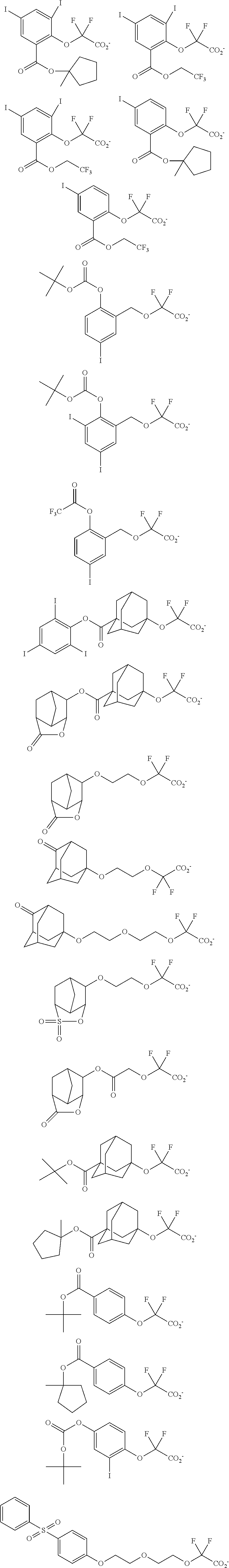

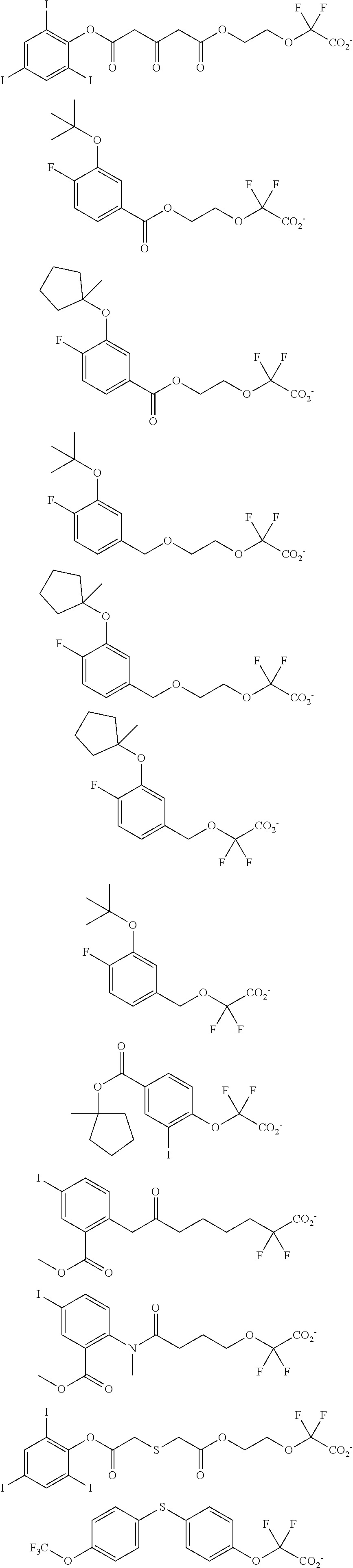

Of these, the following anions are preferred.

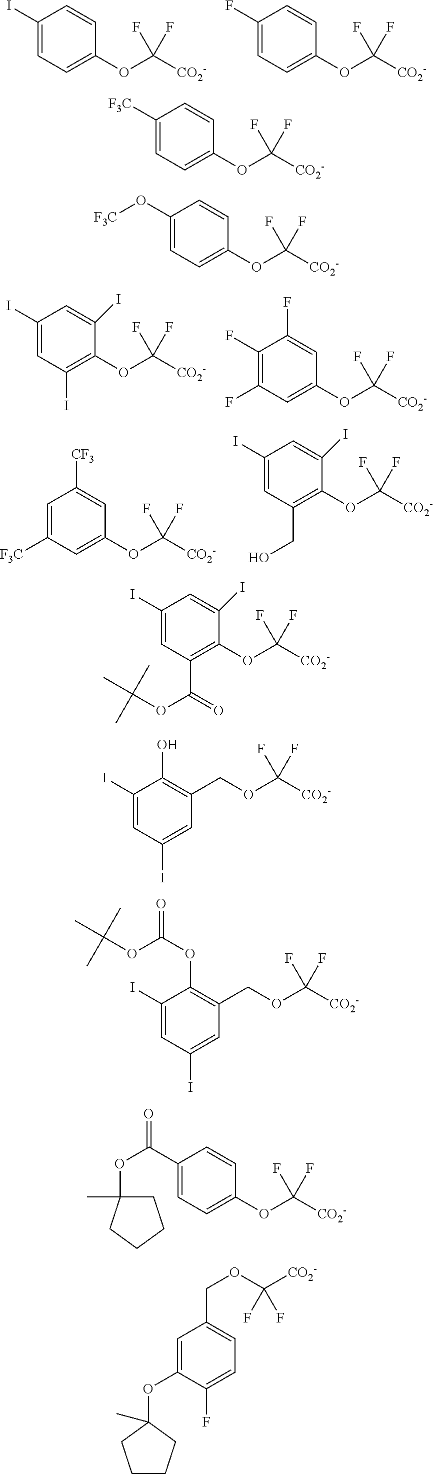

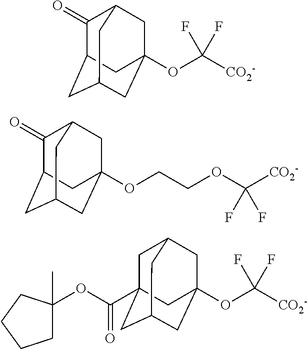

Exemplary structures for the onium salt compound of the invention include arbitrary combinations of cations with anions, both as exemplified above.

The onium salt compound of formula (1) may be synthesized, for example, according to the following scheme.

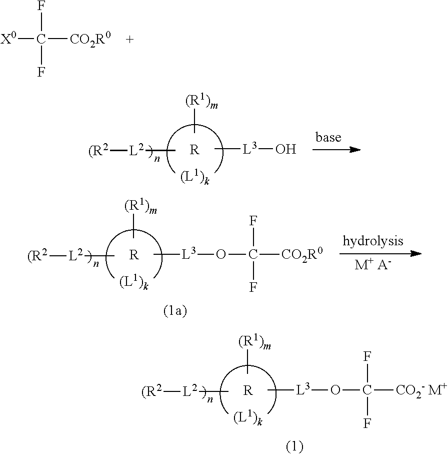

Herein R¹, R², L¹, L², L³, R, m, n, k, and M⁺ are as defined above. X⁰ is chlorine, bromine or iodine. R⁰ is a C₁-C₅ hydrocarbyl group. A⁻ is an anion.

The first step is a nucleophilic substitution reaction of an α-haloacetate with an alcohol in the presence of a base to synthesize an intermediate compound (1a). The haloacetate compounds wherein X⁰ is chlorine or bromine and R⁰ is methyl or ethyl are commercially available. Examples of the base used herein include organic bases such as triethylamine, diisopropylethylamine, pyridine, 2,6-lutidine, and diazabicycloundecene, and inorganic bases such as sodium carbonate, potassium carbonate, cesium carbonate, sodium hydroxide, potassium hydroxide, sodium hydride, and potassium hydride.

The nucleophilic substitution reaction may be performed under suitable conditions, typically in a solvent, preferably an aprotic polar solvent such as dimethyl sulfoxide, N,N-dimethylformamide, or N-methylpyrrolidone, and at a temperature from 40° C. to the boiling point of the solvent. When the alcohol has a functional group which is unstable under the reaction conditions, or a reactive site in addition to the desired hydroxyl group, intermediate compound (1a) is obtainable by etherifying the alcohol in the protected state and effecting deprotection reaction.

In the second step, intermediate compound (1a) is hydrolyzed in a standard way to cleave the ester moiety R⁰. The resulting carboxylate or carboxylic acid is subjected to salt exchange with an onium salt of the desired cation having the formula: M⁺A⁻, whereby the desired onium salt compound (1) is synthesized. It is noted that A⁻ is preferably a chloride, bromide, iodide, methylsulfate or methanesulfonate anion because exchange reaction takes place in a quantitative manner. The salt exchange in the second step is readily accomplished by any well-known method, for example, with reference to JP-A 2007-145797.

The synthesis method mentioned above is merely exemplary and the method is not limited thereto.

A chemically amplified resist composition comprising the inventive onium salt compound is improved in sensitivity, LWR and CDU. Although the detail is not well understood, the following reason is presumed. The onium salt compound has a carboxylate anion which is substituted with fluorine at α-position. As compared with conventional acid diffusion inhibitors of carboxylic salt type, the conjugated acid has a high acidity, providing a high sensitivity. As compared with acid diffusion inhibitors of alkanesulfonic acid type having a similarly high acidity, the onium salt compound has a satisfactory quenching ability, which leads to improvements in lithography performance like LWR and CDU.

The inventive onium salt compound is characterized by having a carbonyl group, ester bond, sulfinyl group, sulfonyl group or sulfonate bond. These groups have a superior acid diffusion suppression ability to ether and thioether bonds. Therefore, the chemically amplified resist composition comprising the inventive onium salt compound exhibits a high contrast and is improved in lithography performance. In processing by the EUV lithography, these groups are effective for restraining the diffusion of secondary electrons, as compared with hydroxyl groups, ether bonds, and thioether bonds. Particularly when the carbonyl carbon or sulfonyl sulfur of these groups bonds to an aromatic ring, the conjugated system is elongated to restrain the diffusion of secondary electrons to a greater extent. Since the acid diffusion is thus restrained, there can be formed a pattern with improvements in lithography performance.

In the EUV lithography involving exposure to high-energy radiation, some ester bonds or sulfonate bonds can be cleaved. Now that the inventive onium salt compound has an ester bond attached to the ring R via carbonyl carbon or a sulfonate bond attached to the ring R via sulfur, upon occurrence of cleavage of a bond, a carboxylic acid or sulfonic acid is generated on the anion matrix side, from which a high contrast and improvements in lithography performance are expectable. Also, when hydrolytic reaction occurs in part during development in an alkaline developer, the inventive onium salt compound having an ester bond or sulfonate bond is accompanied by the creation of a carboxylate or sulfonate on the matrix side, whereby developer solubility is improved and development defects are reduced. Equivalent effects are expectable even when the ring R is a lactone or sultone ring.

The inventive onium salt compound contains halogen, trifluoromethyl or trifluoromethoxy in its anion. Halogen atoms are known for efficient absorption of EUV as compared with hydrogen, carbon, nitrogen and oxygen atoms. Thus a chemically amplified resist composition comprising the inventive onium salt compound having halogen, especially iodine exhibits a high sensitivity in the EUV lithography. Iodine, which is an atom of large size, is sterically bulky, from which an acid diffusion-suppressing effect is expectable. The trifluoromethyl or trifluoromethoxy group is also sterically bulky and efficiently absorptive to EUV due to the inclusion of three fluorine atoms, from which a high sensitivity and an acid diffusion-suppressing effect are expectable. Therefore, the chemically amplified resist composition comprising the inventive onium salt compound having halogen, trifluoromethyl or trifluoromethoxy in its anion, when processed by the EUV lithography, exhibits a high sensitivity, controlled acid diffusion, and improved lithography performance.

Patent Document 1 discloses anions having the following formulae (a) to (e).

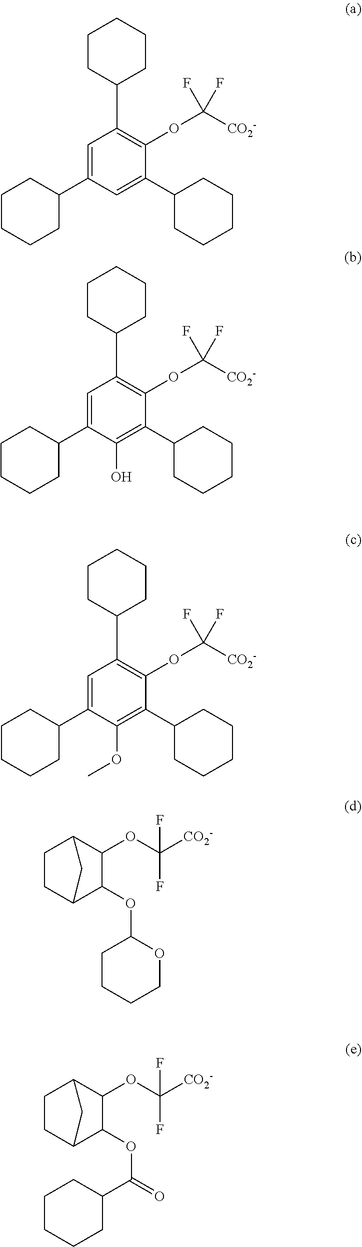

Since the anions of formulae (a) to (e) do not possess a partial structure (carbonyl group, ester bond, sulfinyl group, sulfonyl group or sulfonate bond) which is specific to the present invention, they exert a poor acid diffusion-suppressing effect in the EUV lithography, as compared with the anion possessing the specific partial structure. They exhibit a low sensitivity as compared with the anion having halogen, trifluoromethyl or trifluoromethoxy. Also, the anion of formula (e) has an ester bond, but its bonding direction is reverse to the ester bond in the inventive onium salt compound. When the bond is cleaved upon exposure or development, the anion of formula (e) generates a hydroxyl group, which is inferior in defectiveness and other properties as compared with the inventive onium salt compound designed to generate a carboxylic acid (anion) or sulfonic acid (anion). Further, since the anions of formulae (a) to (e) do not contain halogen, trifluoromethyl or trifluoromethoxy, they exhibit a low sensitivity in the EUV lithography as compared with the inventive onium salt compound. As compared with the salt compounds described in Patent Document 1, the inventive onium salt compound containing the specific partial structure is excellent in lithography properties. Such outstanding effects are unexpectable from Patent Document 1.

### Chemically Amplified Resist Composition

Another embodiment of the invention is a chemically amplified resist composition comprising (A) a base polymer adapted to change its solubility in a developer under the action of an acid, (B) a photoacid generator, (C-1) an acid diffusion inhibitor comprising the inventive onium salt compound, and (D) an organic solvent as essential components, and if necessary, (C-2) an acid diffusion inhibitor other than the inventive onium salt compound, (E) a surfactant, and (F) other components.

A further embodiment of the invention is a chemically amplified resist composition comprising (A′) a base polymer adapted to change its solubility in a developer under the action of an acid, the base polymer comprising recurring units having a function of generating an acid upon exposure to light, (C-1) an acid diffusion inhibitor comprising the inventive onium salt compound, and (D) an organic solvent as essential components, and if necessary, (B) a photoacid generator, (C-2) an acid diffusion inhibitor other than the inventive onium salt compound, (E) a surfactant, and (F) other components.

### (A) Base Polymer

Component (A) is a base polymer adapted to change its solubility in a developer under the action of an acid. It is preferably a polymer comprising recurring units having the formula (a) or recurring units having the formula (b), which are also referred to as recurring units (a) and (b), respectively.

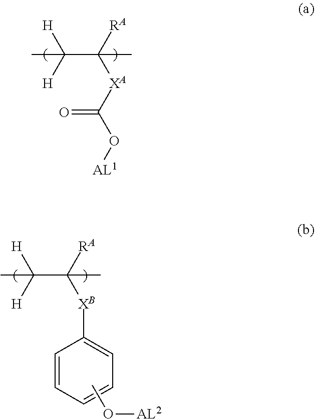

In formulae (a) and (b), R^{A} is hydrogen or methyl. X^{A} is a single bond, phenylene group, naphthylene group or (backbone)-C(═O)—O—X^{A1}—, wherein X^{A1} is a C₁-C₁₅ hydrocarbylene group which may contain a hydroxyl moiety, ether bond, ester bond or lactone ring. X^{B} is a single bond or ester bond. AL¹ and AL² are each independently an acid labile group. The hydrocarbylene group may be saturated or unsaturated and straight, branched or cyclic.

While the acid labile groups AL¹ and AL² are not particularly limited, suitable acid labile groups include C₄-C₂₀ tertiary hydrocarbyl groups, trialkylsilyl groups in which each alkyl moiety has 1 to 6 carbon atoms, and C₄-C₂₀ oxoalkyl groups. With respect to the structure of these acid labile groups, reference should be made to U.S. Pat. No. 9,164,384 (JP-A 2014-225005, paragraphs [0016]-[0035]).

Acid labile groups having the following formula (L1) are preferred as AL¹ and AL².

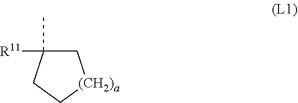

In formula (L1), R¹¹ is a C hydrocarbyl group in which —CH₂— may be replaced by —O—, and “a” is 1 or 2.

Of the acid labile groups AL¹ and AL², the following groups are most preferred.

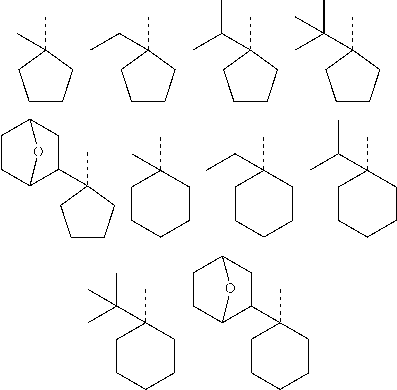

A resist composition comprising a base polymer containing recurring units (a) or (b) having an acid labile group and the inventive onium salt compound is improved in lithography performance. Although the detail is not well understood, the following reason is presumed. When a tertiary alicyclic hydrocarbyl group having formula (L1) is bonded to the ester site, the group becomes more acid labile or decomposable due to steric repulsion than other chainlike tertiary alkyl groups such as tert-butyl and tert-pentyl. Also, as compared with acid labile groups having adamantane ring, the acid labile group having formula (L1) allows for easy progress of acid-aided elimination reaction, tending to provide a high sensitivity. Therefore, when a tertiary alicyclic hydrocarbyl group is incorporated in the polarity switch unit of the base polymer in a resist composition, the dissolution contrast between exposed and unexposed regions is increased. While the inventive onium salt compound serves as an acid diffusion inhibitor, the carboxylic acid generated after quenching of a strong acid has a relatively high acidity. When the inventive onium salt compound is used in combination with acid labile group units having high reactivity, the acid generated after quenching promotes elimination reaction, though to a slight extent, leading to an improvement in contrast. As a result, lithography performance is improved. Although the acid labile group of tertiary ether type as represented by formula (b) is typically low in acid-aided elimination reactivity, the elimination reaction is promoted in the co-presence of a protonic hydroxyl group having high acidity like phenol. As a result, there are obtained similar effects to the aforementioned tertiary ester type.

Examples of the structure having formula (a) wherein X^{A} is a variant include the structures described in U.S. Pat. No. 9,164,384 (JP-A 2014-225005, paragraph [0015]). Of these, preferred structures are shown below. Herein R^{A} and AL¹ are as defined above.

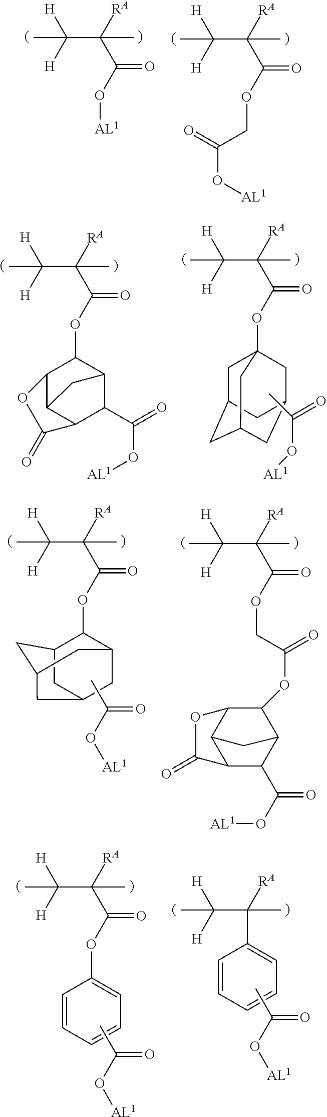

Examples of the recurring unit (a) are given below, but not limited thereto. Herein R^{A} is as defined above.

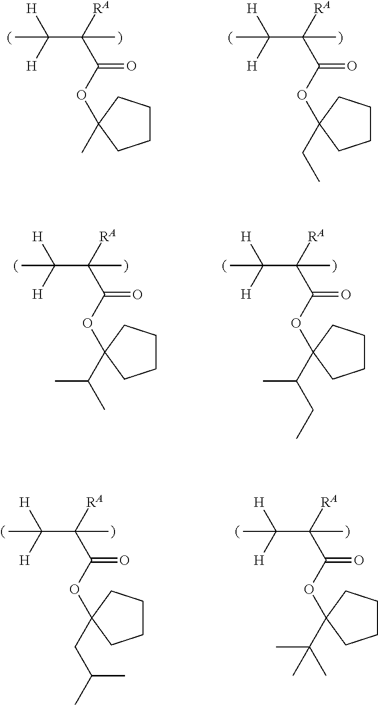

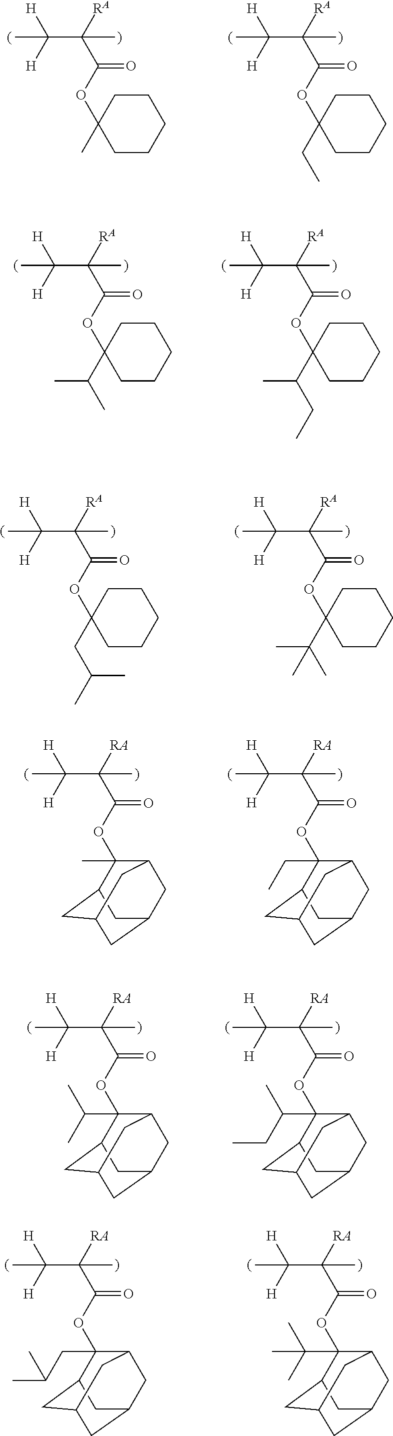

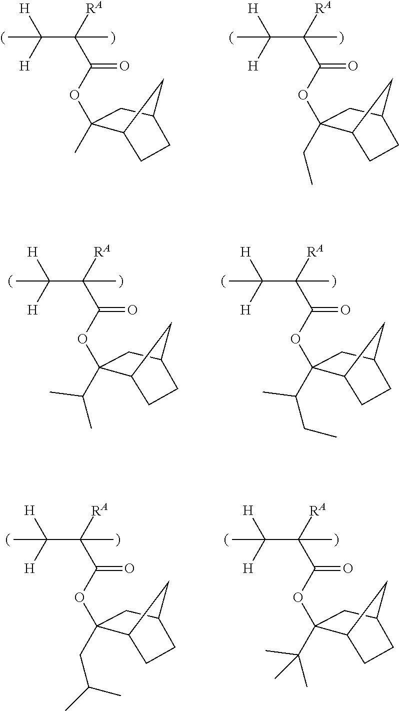

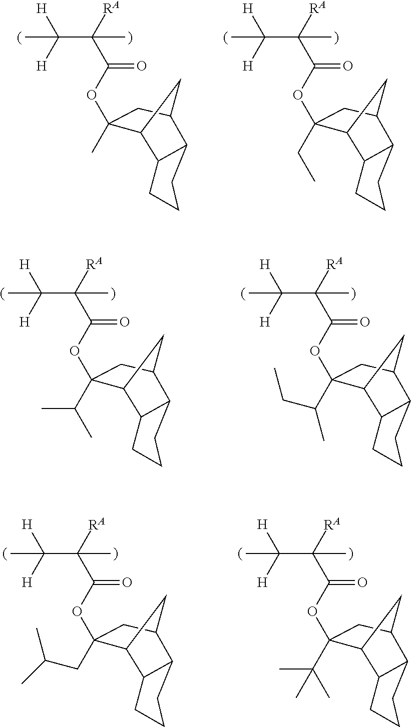

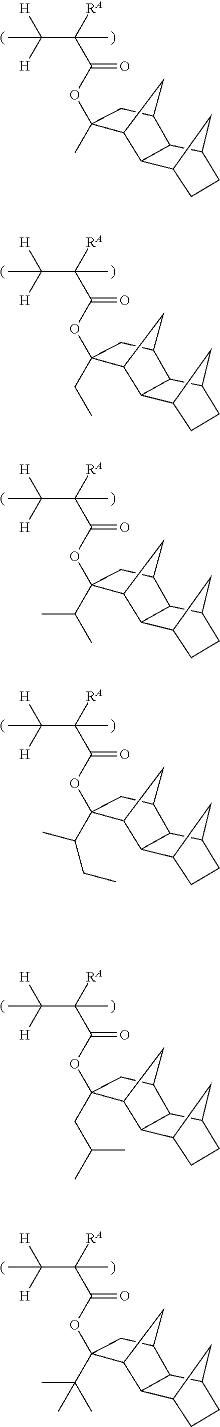

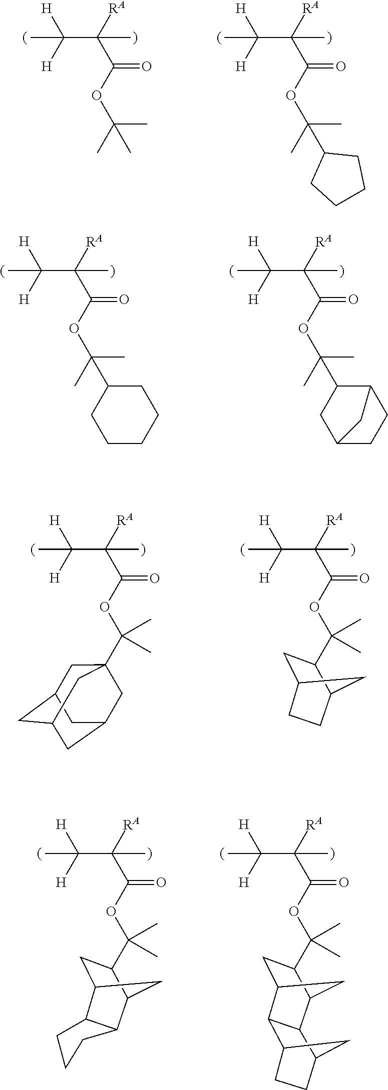

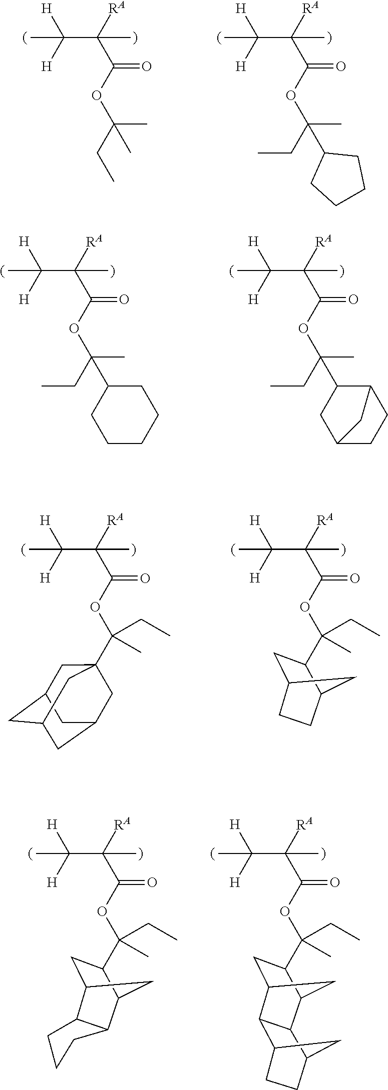

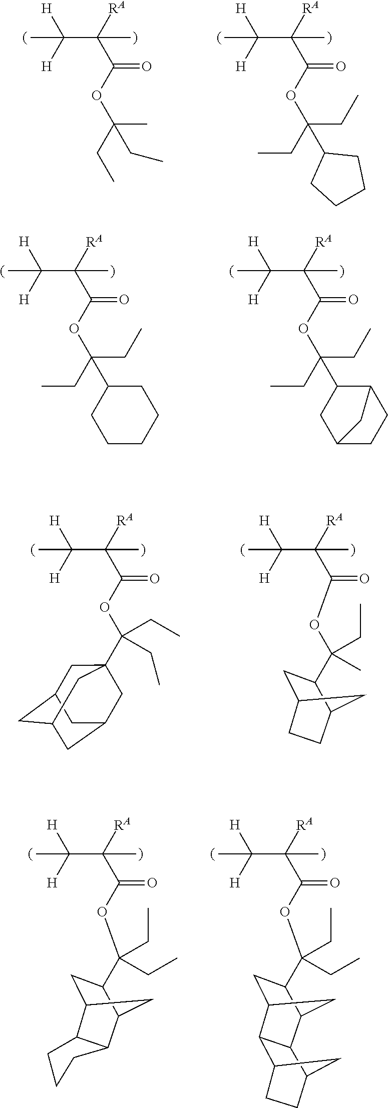

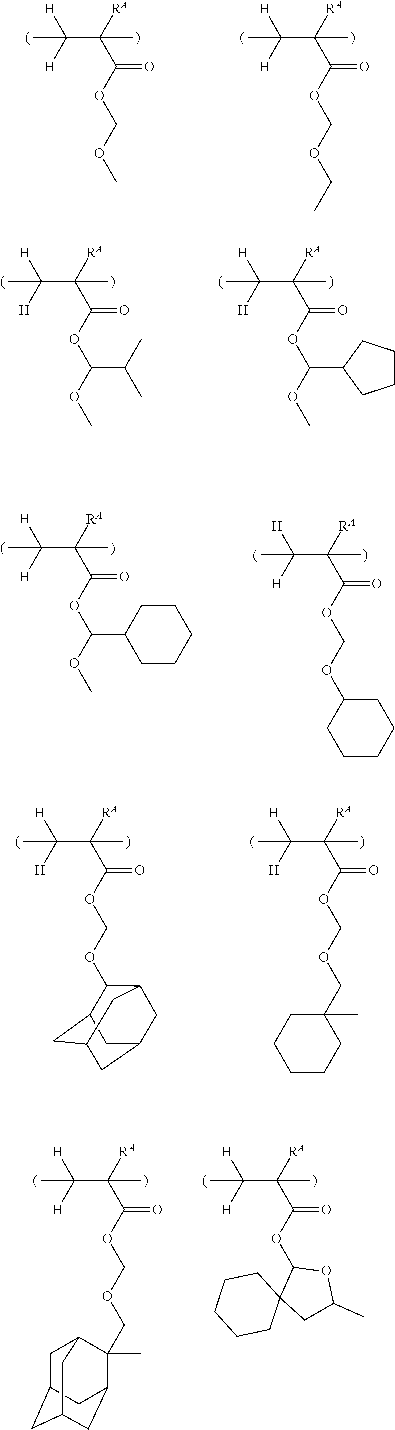

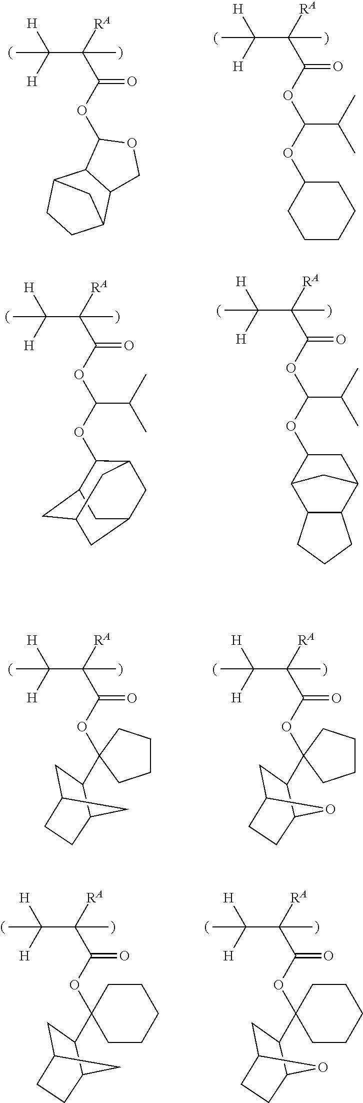

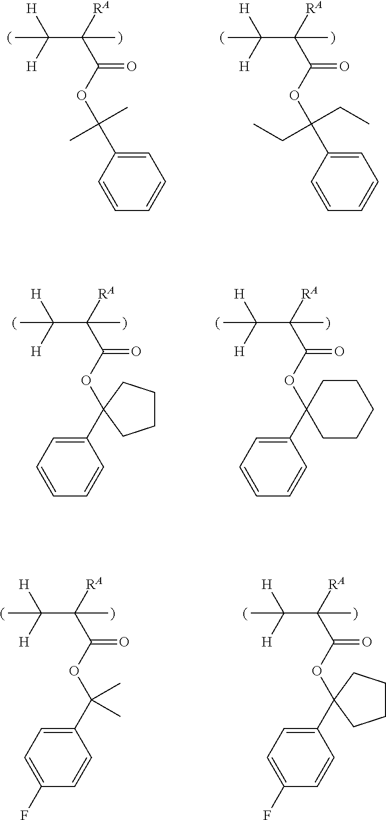

Examples of the recurring unit (b) are given below, but not limited thereto. Herein R^{A} is as defined above.

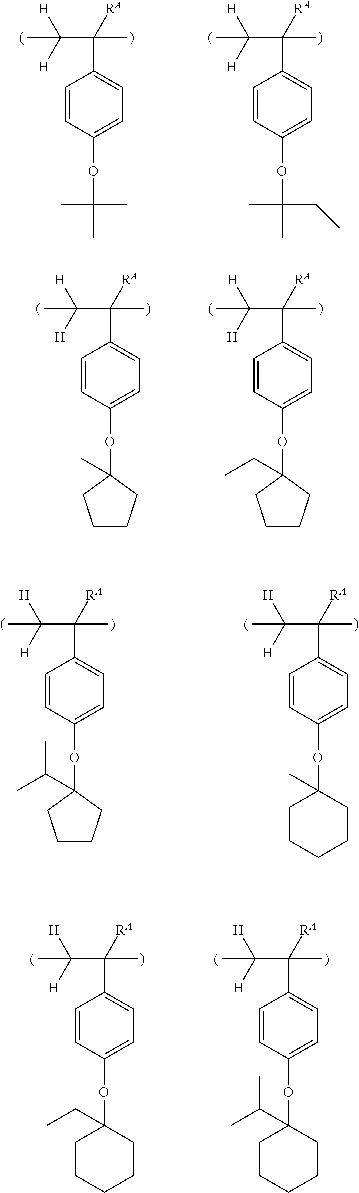

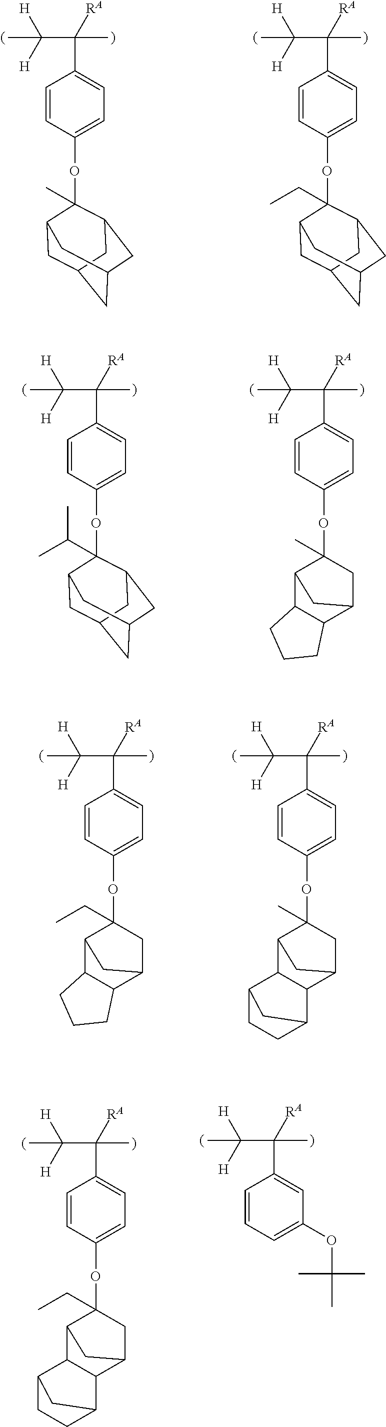

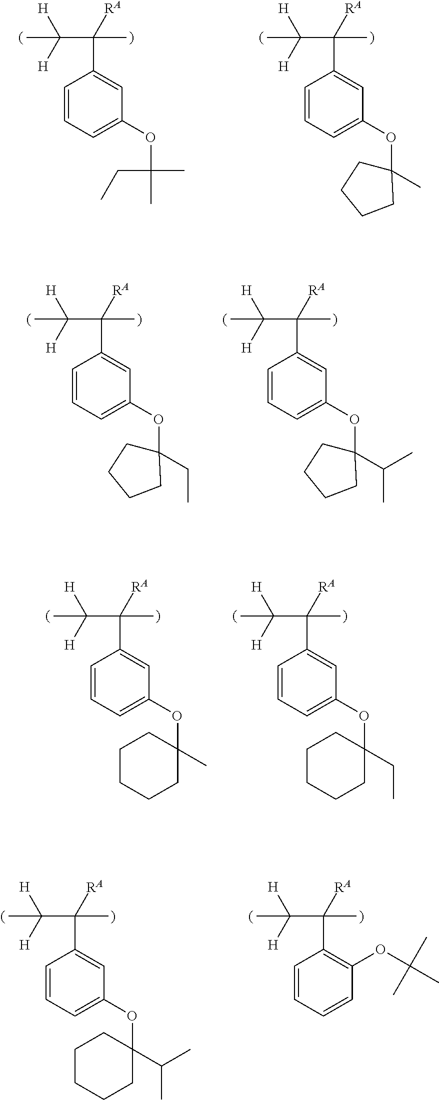

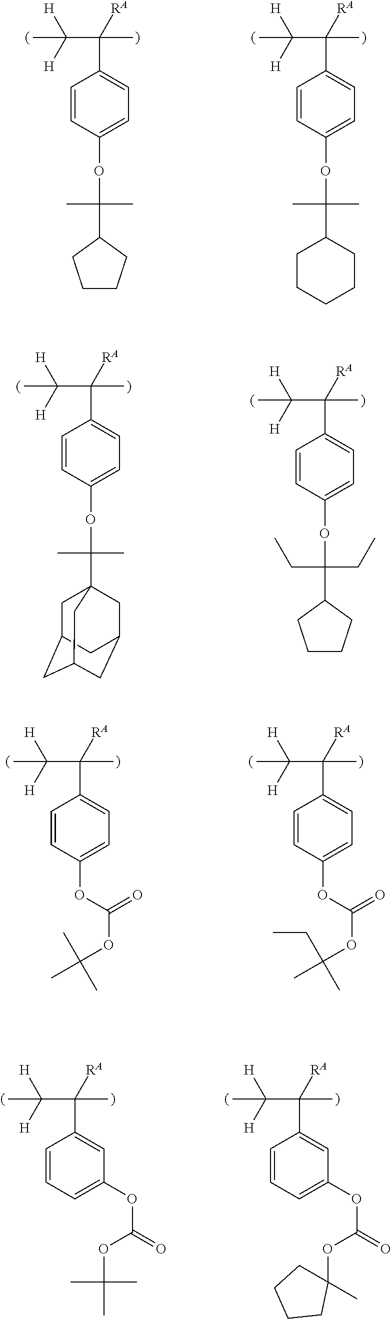

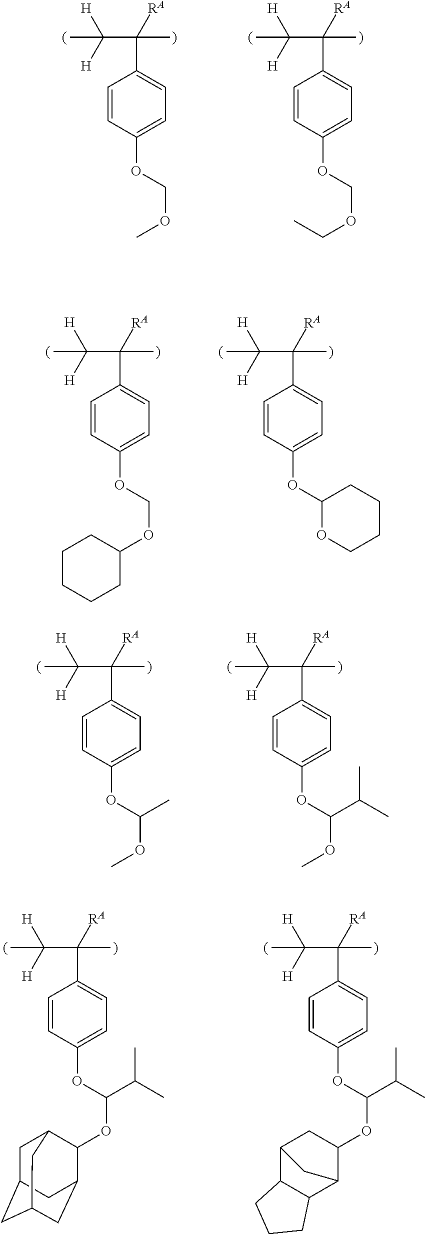

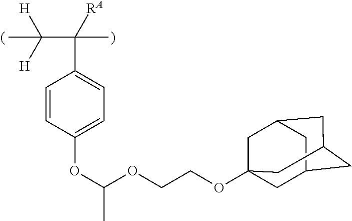

Although the above examples correspond to the unit wherein X^{A} or X^{B} is a single bond, combinations with similar acid labile groups are possible where X^{A} or X^{B} is other than a single bond. Examples of the units wherein X^{A} is other than a single bond are as exemplified above. Examples of the units wherein X^{B} is an ester bond correspond to the above-exemplified units wherein the single bond between the backbone and the benzene ring is replaced by an ester bond.

The base polymer may further comprise recurring units having the formula (c), which are also referred to as recurring units (c).

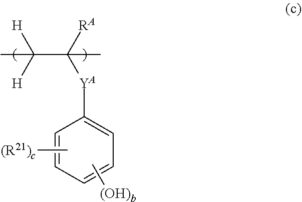

In formula (c), R^{A} is hydrogen or methyl. Y^{A} is a single bond or ester bond.

In formula (c), R²¹ is fluorine, iodine or a C₁-C₁₀ hydrocarbyl group. The hydrocarbyl group may be saturated or unsaturated and straight, branched or cyclic. Examples include alkyl groups such as methyl, ethyl, propyl, isopropyl, n-butyl, sec-butyl, tert-butyl, n-pentyl, n-hexyl, n-octyl, n-nonyl, and n-decyl; cyclic saturated hydrocarbyl groups such as cyclopentyl, cyclohexyl, and adamantyl; aryl groups such as phenyl; and to combinations thereof.

A constituent —CH₂— in the hydrocarbyl group may be replaced by —O— or —C(═O)—. The constituent —CH₂— in the hydrocarbyl group may be one bonding to a carbon atom on the benzene ring in formula (c). Examples of the substituted hydrocarbyl group include, but are not limited to, methoxy, ethoxy, propoxy, butoxy, phenoxy, 2-methoxyethoxy, acetyl, ethylcarbonyl, hexylcarbonyl, acetoxy, ethylcarbonyloxy, propylcarbonyloxy, pentylcarbonyloxy, hexylcarbonyloxy, heptylcarbonyloxy, methoxymethylcarbonyloxy, (2-methoxyethoxy)methylcarbonyloxy, methyloxycarbonyl, ethyloxycarbonyl, hexyloxycarbonyl, phenyloxycarbonyl, acetoxymethyl, phenoxymethyl, and methoxycarbonyloxy. Preferably R²¹ is fluorine, iodine, methyl, acetyl or methoxy.

In formula (c), b is an integer of 1 to 5, c is an integer of 0 to 4, and b+c is 1 to 5. Preferably bis 1, 2 or 3, and c is 0, 1 or 2.

The recurring unit (c) serves to improve the adhesion to the substrate or the underlay film. Since the recurring unit (c) has a phenolic hydroxyl group with high acidity, it promotes the action of an acid generated upon exposure, contributing to a higher sensitivity, and becomes a proton source to the acid generated upon EUV exposure, from which an improvement in sensitivity is expectable.

Examples of the recurring unit (c) are given below, but not limited thereto. Herein R^{A} is as defined above.

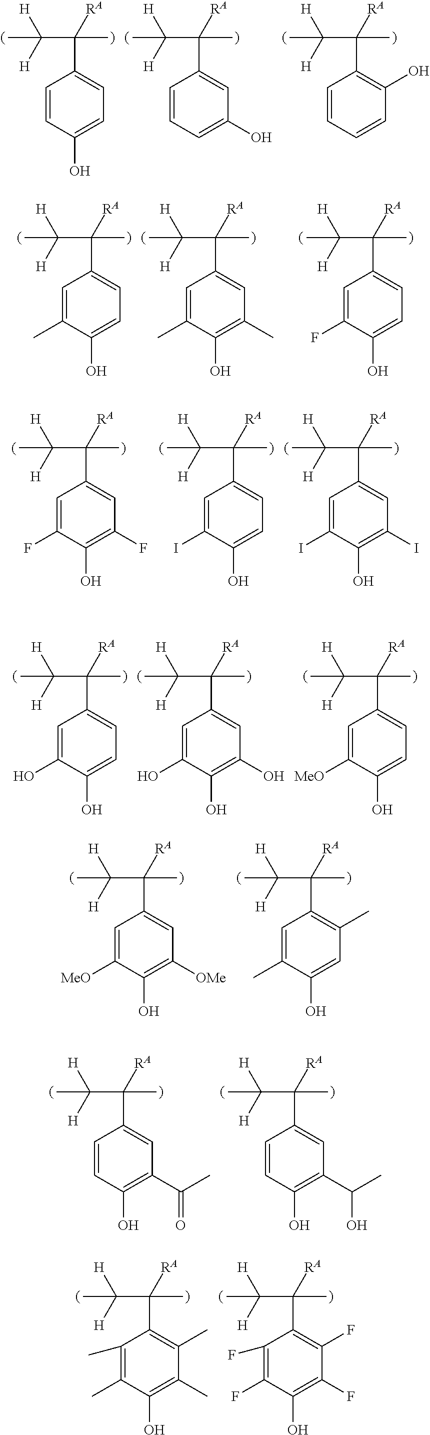

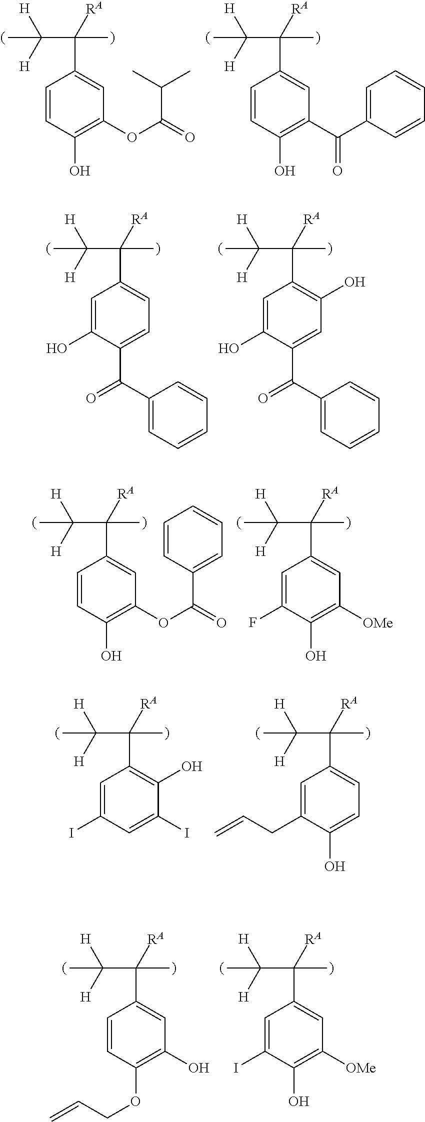

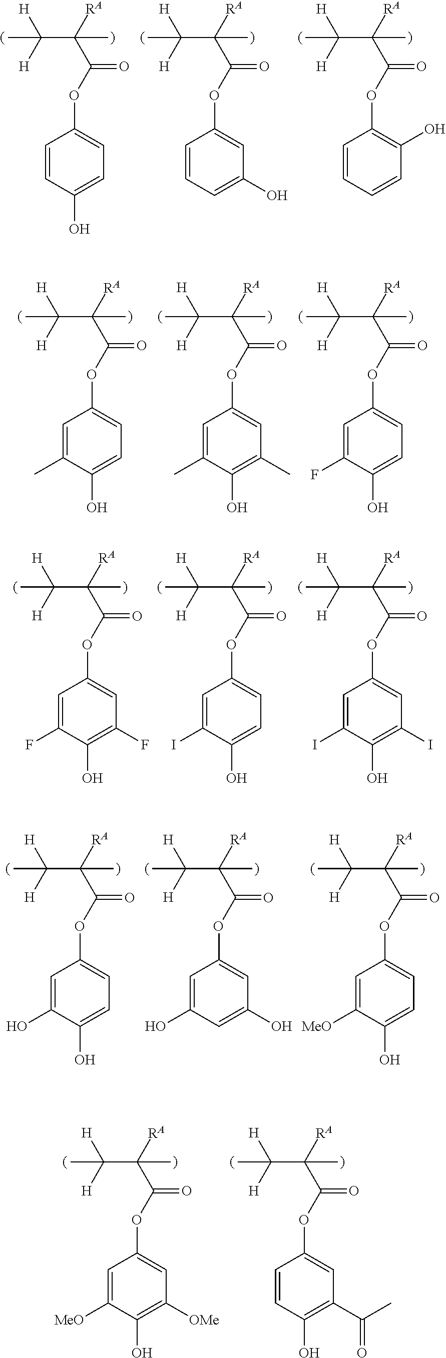

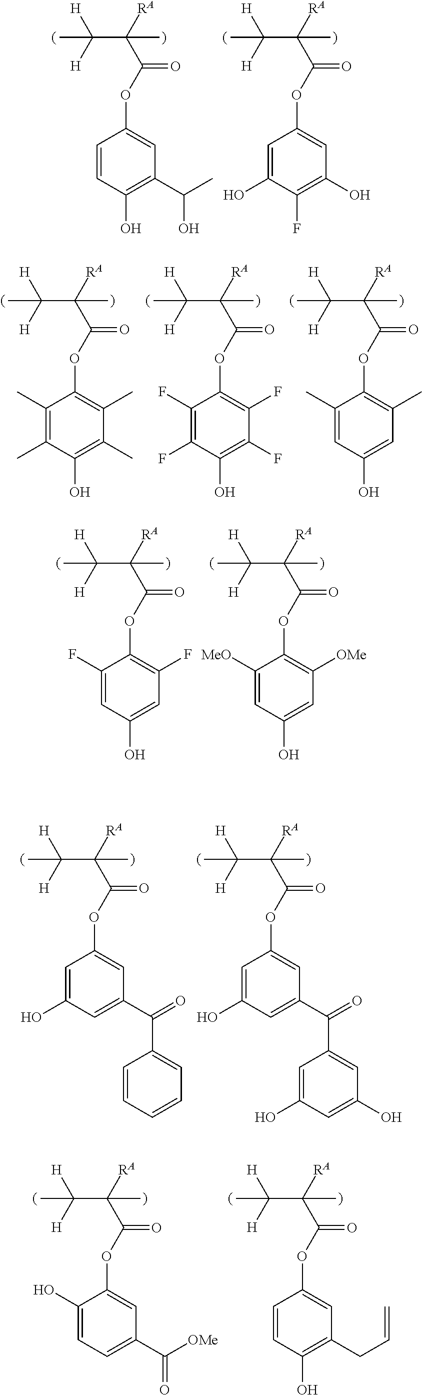

Of the above recurring units (c), the following units are preferred. Herein R^{A} is as defined above.

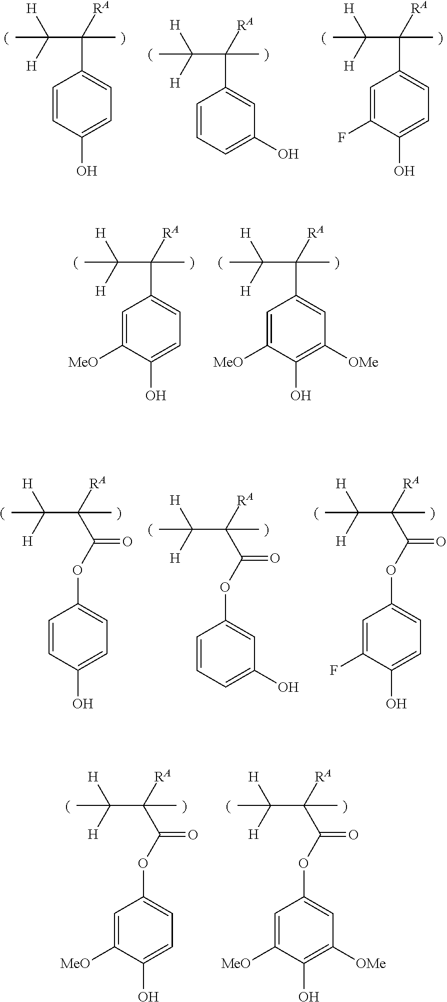

The base polymer may further comprise recurring units having the formula (d1), (d2), (d3) or (d4), which are also referred to as recurring units (d1) to (d4), respectively.

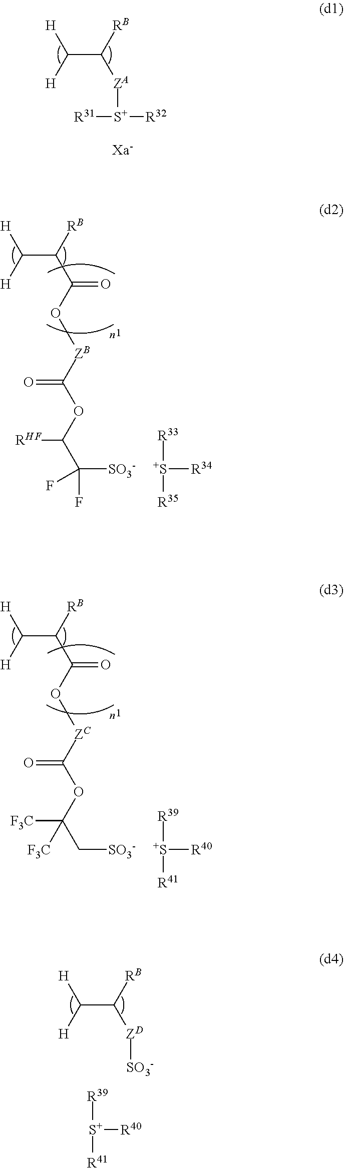

In formulae (d1) to (d4), R^{B} is hydrogen, fluorine, methyl or trifluoromethyl. Z^{A} is a single bond, phenylene, —O—Z^{A1}—, —C(═O)—O—Z^{A1}— or —C(═O)—NH—Z^{A1}—, wherein Z^{A1} is a C₁-C₂₀ hydrocarbylene group which may contain a heteroatom. Z^{B} and Z^{C} are each independently a single bond or a C₁-C₂₀ hydrocarbylene group which may contain a heteroatom. Z^{D} is a single bond, methylene, ethylene, phenylene, fluorinated phenylene, —O—Z^{D1}—, —C(═O)—O—Z^{D1}— or —C(═O)—NH—Z^{D1}—, wherein Z^{D1} is an optionally substituted phenylene group.

The hydrocarbylene group represented by Z^{A1} may be saturated or unsaturated and straight, branched or cyclic. Examples thereof include alkanediyl groups such as methylene, ethane-1,1-diyl, ethane-1,2-diyl, propane-1,2-diyl, propane-1,3-diyl, butane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, and 2,2-dimethylpropane-1,3-diyl; cyclic saturated hydrocarbylene groups such as cyclopentanediyl, cyclohexanediyl, norbornanediyl, and adamantanediyl; alkenediyl groups such as ethene-1,2-diyl, 1-propene-1,3-diyl, 2-butene-1,4-diyl, and 1-methyl-1-butene-1,4-diyl; unsaturated alicyclic hydrocarbylene groups such as 2-cyclohexene-1,4-diyl; aromatic hydrocarbylene groups such as phenylene and naphthylene, and combinations thereof. In these groups, some or all of the hydrogen atoms may be substituted by a moiety containing a heteroatom such as oxygen, sulfur, nitrogen or halogen, and a moiety containing a heteroatom such as oxygen, sulfur or nitrogen may intervene in a carbon-carbon bond, so that the group may contain a hydroxyl moiety, cyano moiety, carbonyl moiety, ether bond, ester bond, sulfonic acid ester bond, carbonate bond, lactone ring, sultone ring, carboxylic anhydride or haloalkyl moiety.

The hydrocarbylene groups represented by Z^{B} and Z^{C} may be saturated or unsaturated and straight, branched or cyclic. Examples thereof are as exemplified above for the hydrocarbylene group Z^{A1}. Preferably Z^{B} and Z^{C} each are a single bond, adamantanediyl or phenylene.

In formulae (d1) to (d4), R³¹ to R⁴¹ are each independently a C₁-C₂₀ hydrocarbyl group which may contain a heteroatom. The hydrocarbyl group may be saturated or unsaturated and straight, branched or cyclic. Examples include alkyl groups such as methyl, ethyl, n-propyl, isopropyl, n-butyl, sec-butyl, tert-butyl, n-pentyl, n-hexyl, n-octyl, 2-ethylhexyl, n-nonyl, n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, adamantylmethyl; alkenyl groups such as vinyl, allyl, propenyl, butenyl, and hexenyl; unsaturated alicyclic hydrocarbyl groups such as cyclohexenyl; aryl groups such as phenyl, naphthyl, thienyl, 4-hydroxyphenyl, 4-methoxyphenyl, 3-methoxyphenyl, 2-methoxyphenyl, 4-ethoxyphenyl, 4-tert-butoxyphenyl, 3-tert-butoxyphenyl, 2-methylphenyl, 3-methylphenyl, 4-methylphenyl, 4-ethylphenyl, 4-tert-butylphenyl, 4-n-butylphenyl, 2,4-dimethylphenyl, 2,4,6-triisopropylphenyl, methylnaphthyl, ethylnaphthyl, methoxynaphthyl, ethoxynaphthyl, n-propoxynaphthyl, n-butoxynaphthyl, dimethylnaphthyl, diethylnaphthyl, dimethoxynaphthyl, diethoxynaphthyl; aralkyl groups such as benzyl, 1-phenylethyl and 2-phenylethyl, and combinations thereof. In the hydrocarbyl group, some or all of the hydrogen atoms may be substituted by a moiety containing a heteroatom such as oxygen, sulfur, nitrogen or halogen, and a moiety containing a heteroatom such as oxygen, sulfur or nitrogen may intervene in a carbon-carbon bond, so that the group may contain a hydroxyl moiety, cyano moiety, carbonyl moiety, ether bond, ester bond, sulfonic acid ester bond, carbonate bond, lactone ring, sultone ring, carboxylic anhydride or haloalkyl moiety.

Z^{A} and R³¹ to R⁴¹ are preferably of a structure containing a phenyl group which is bonded to S⁺ in the formula.

Any two of Z^{A}, R³¹ and R³² may bond together to form a ring with the sulfur atom to which they are attached, any two of R³³, R³⁴ and R³⁵, any two of R³⁶, R³⁷ and R³⁸, or any two of R³⁹, R⁴⁰ and R⁴¹ may bond together to form a ring with the sulfur atom to which they are attached.

In formula (d2), R^{HF} is hydrogen or trifluoromethyl.

In formula (d2), n¹ is 0 or 1, n¹ is 0 when Z^{B} is a single bond. In formula (d3), n² is 0 or 1, n² is 0 when Z^{C} is a single bond.

In formula (d1), Xa⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate; arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; and methide ions such as tris(trifluoromethylsulfonyl)methide and tris(perfluoroethylsulfonyl)methide. Preferred are anions having the formulae (d1-1) and (d1-2).

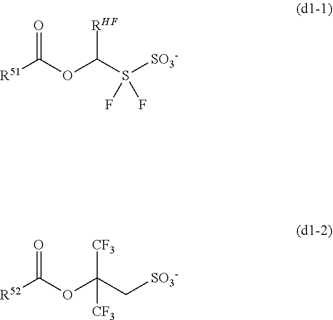

In formulae (d1-1) and (d1-2), R⁵¹ and R⁵² are each independently a C₁-C₄₀ hydrocarbyl group which may contain a heteroatom, and R^{HF} is hydrogen or trifluoromethyl.

Examples of the anion having formula (d1-1) include the anions described in JP-A 2014-177407, paragraphs [0100]-[0101] and the anions shown below, but are not limited thereto. Herein R^{HF} is as defined above.

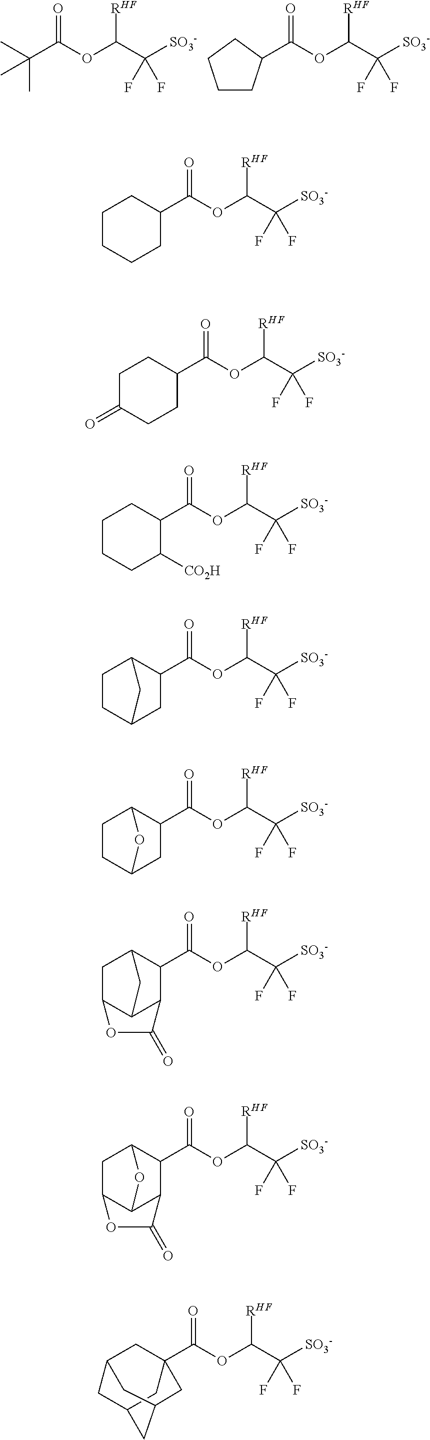

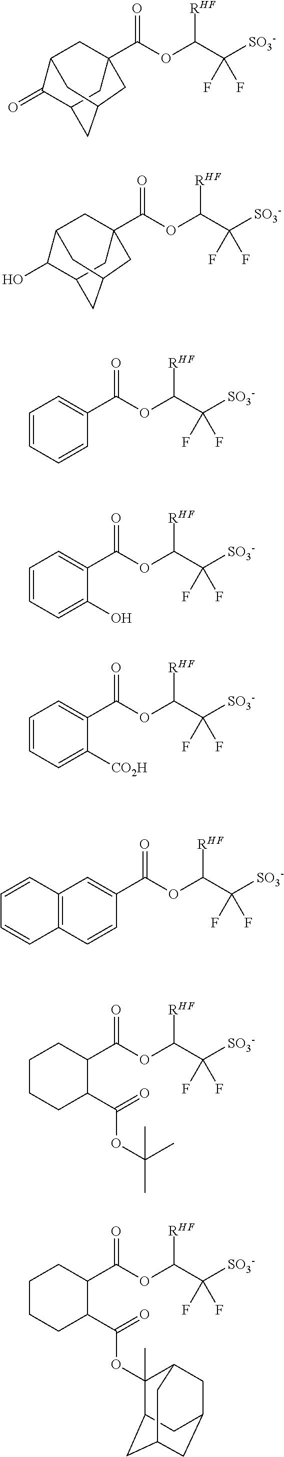

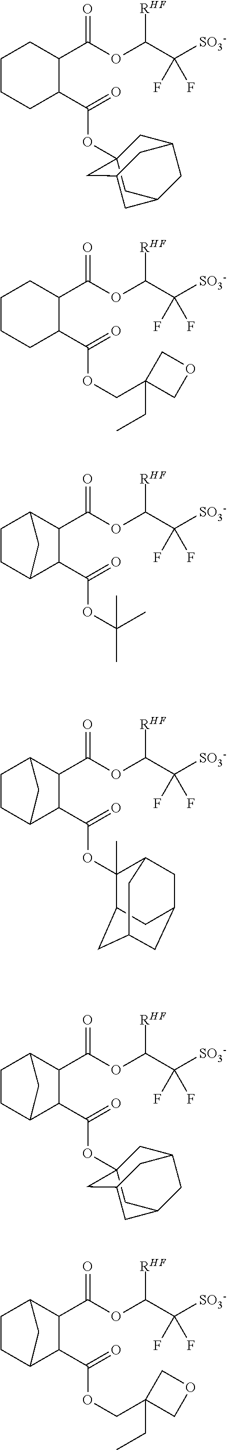

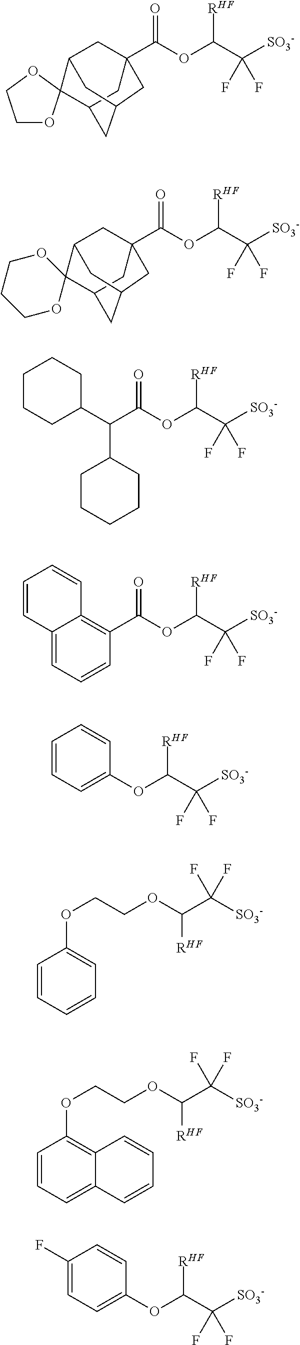

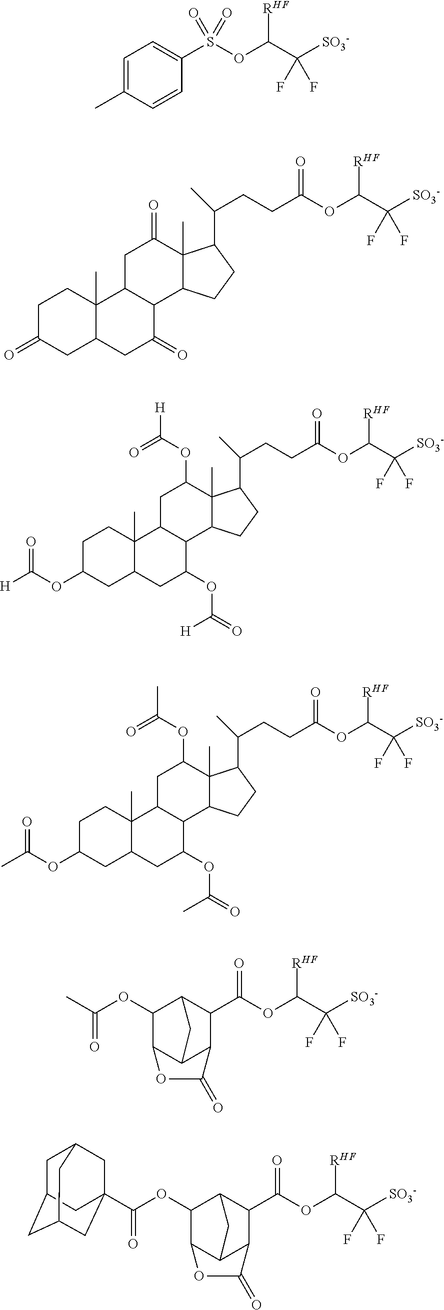

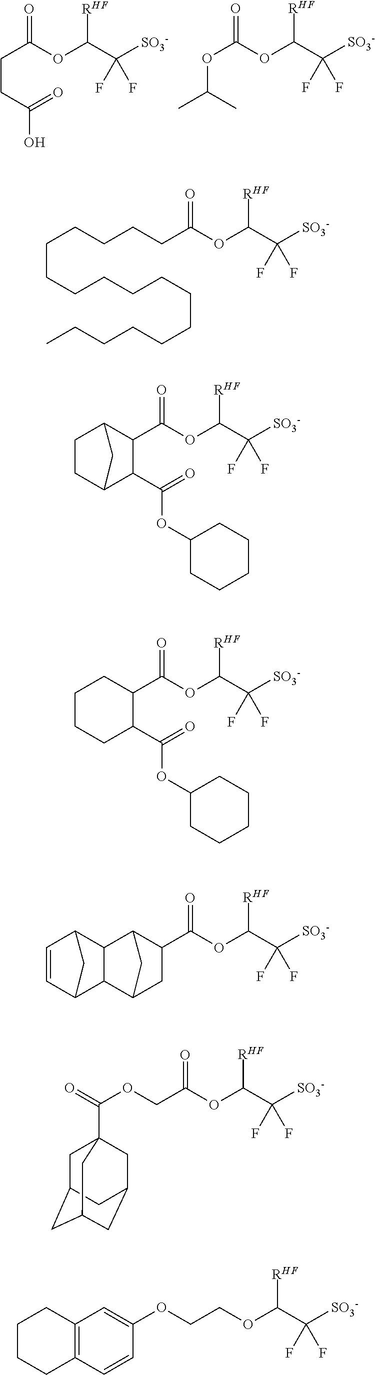

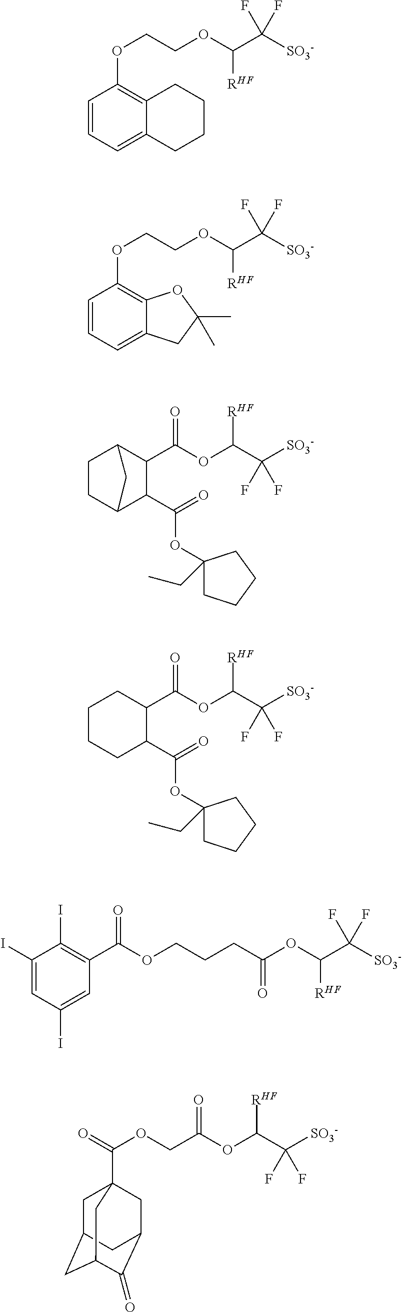

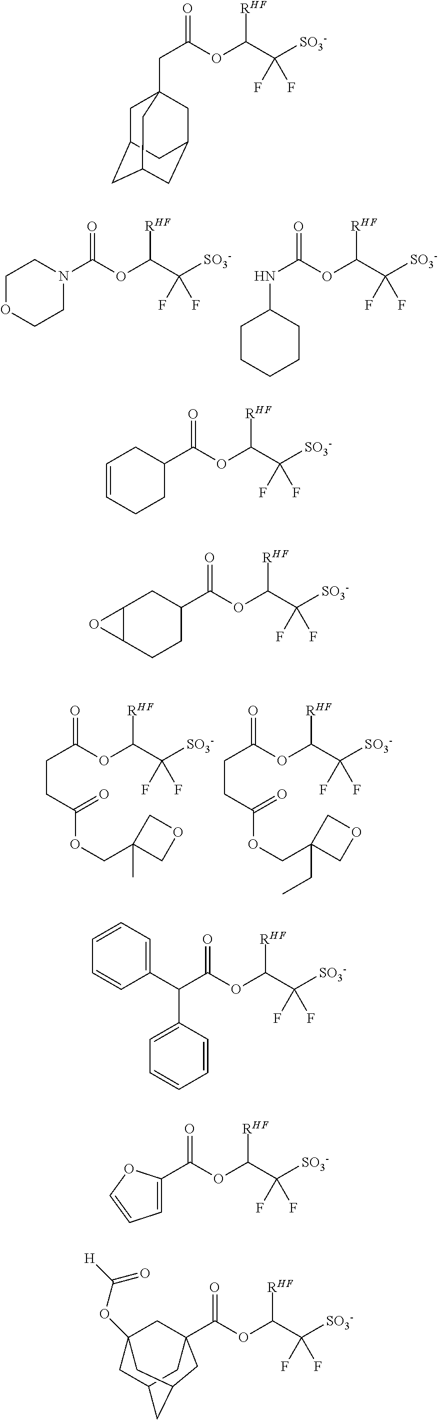

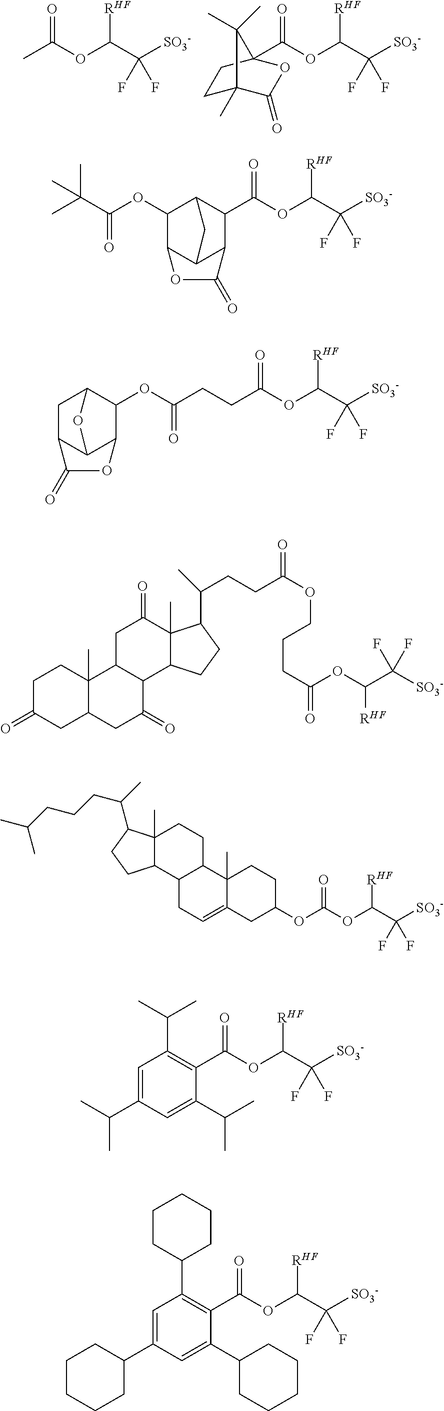

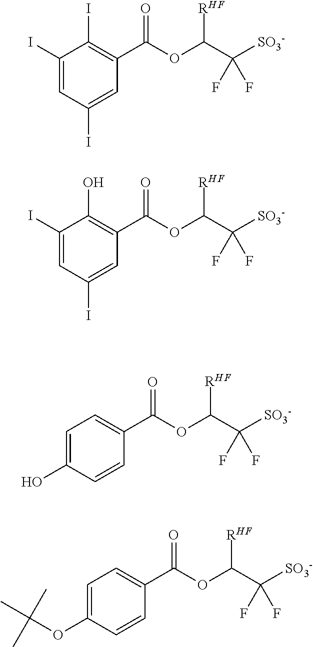

Examples of the anion having formula (d1-2) include the anions described in JP-A 2010-215608, paragraphs [0080]-[10081] and the anions shown below, but are not limited thereto.

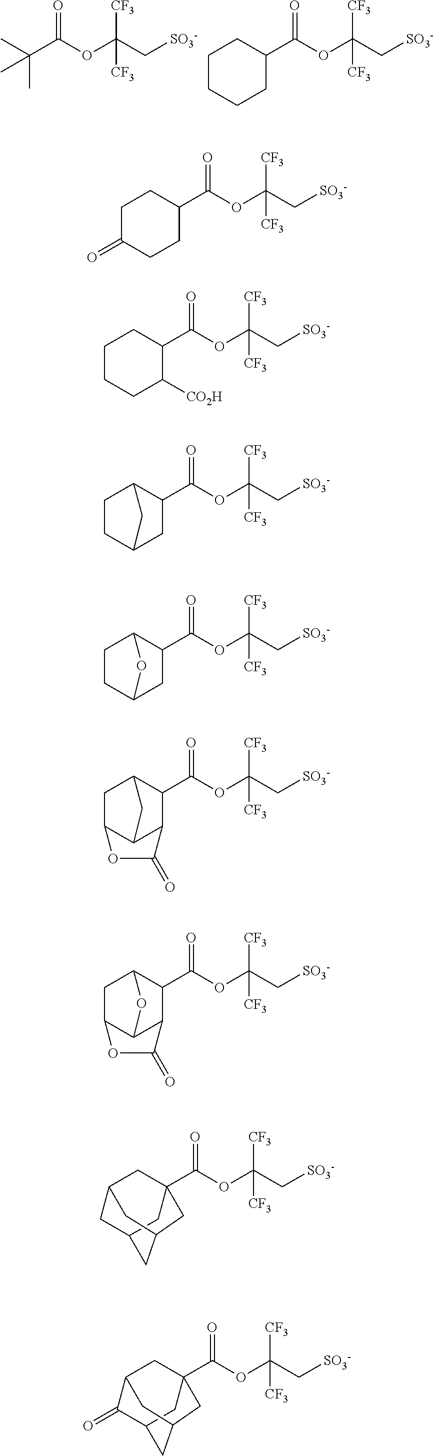

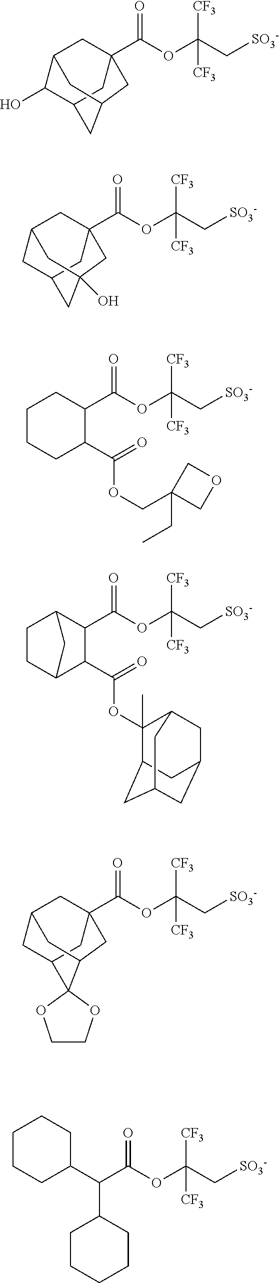

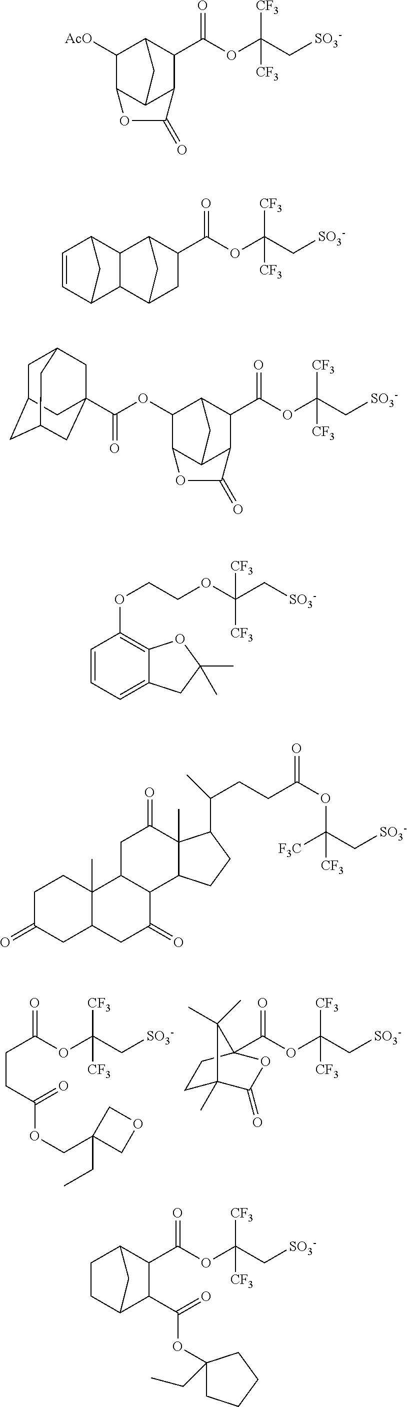

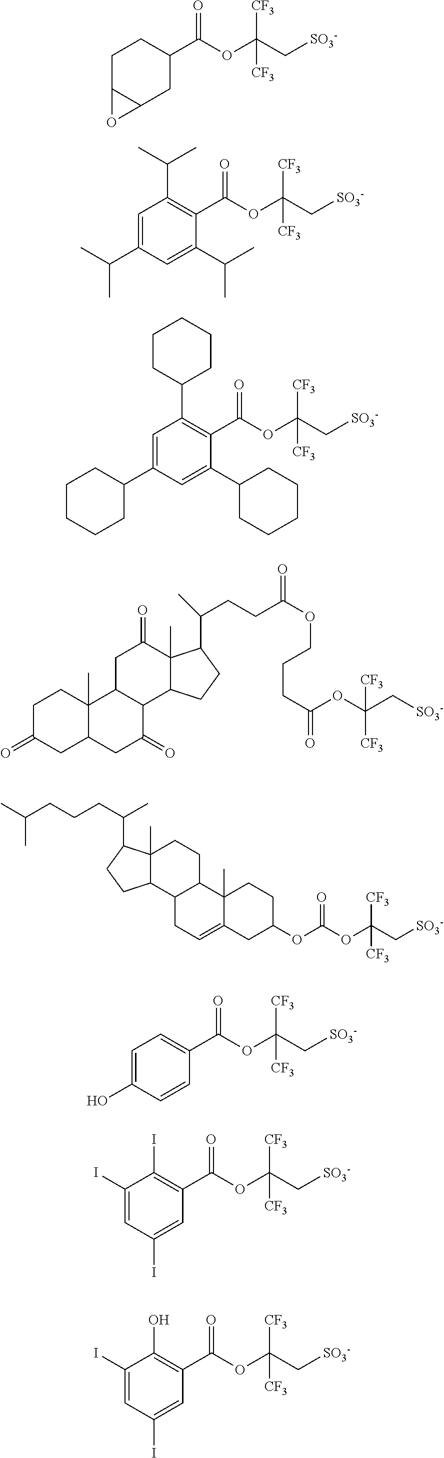

Examples of the anion in recurring unit (d2) include the anions described in JP-A 2014-177407, paragraphs [0021]-[0026]. Exemplary structures of the anion wherein R^{HF} is hydrogen include the anions described in JP-A 2010-116550, paragraphs [0021]-[0028]. Exemplary structures of the anion wherein R^{HF} is trifluoromethyl include the anions described in JP-A 2010-077404, paragraphs [0021]-[0027].

Examples of the anion in recurring unit (d3) correspond to the examples of the anion in recurring unit (d2) wherein —CH(R^{HF})CF₂SO₃⁻ is replaced by —C(CF₃)₂CH₂SO₃⁻.

Preferred examples of the anion in recurring units (d2) to (d4) are given below, but not limited thereto. Herein R^{B} is as defined above.

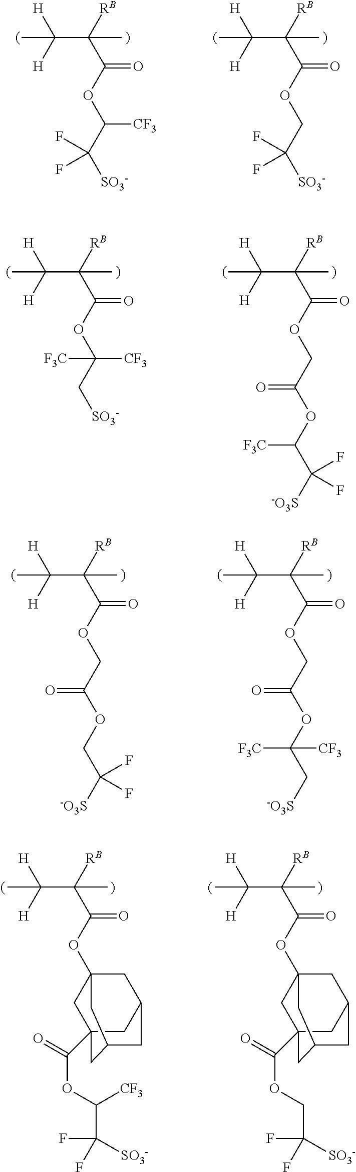

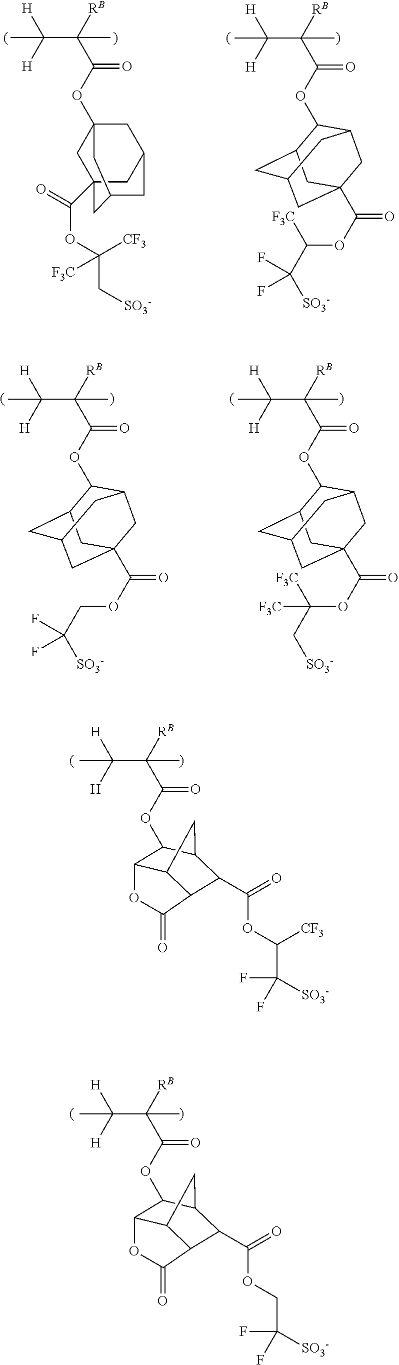

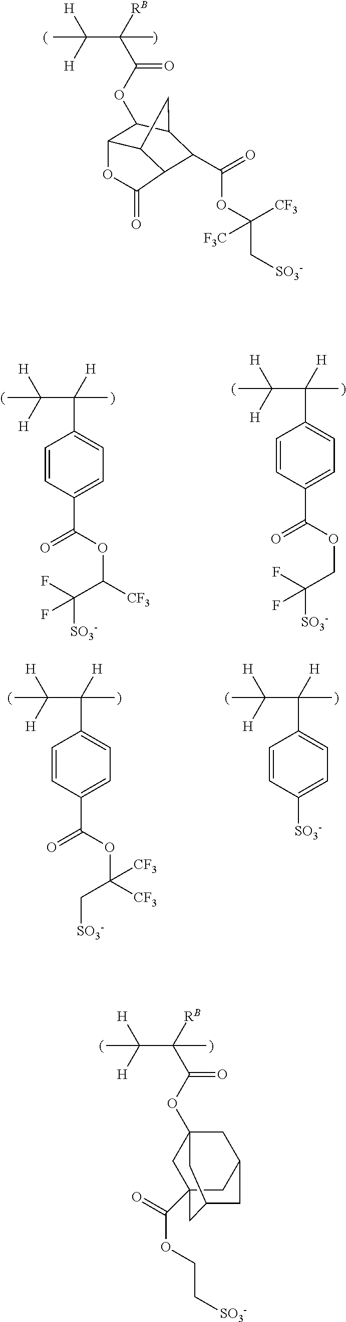

Examples of the sulfonium cation in recurring units (d2) to (d4) include those described in JP-A 2008-158339, paragraph [0223] as well as those exemplified above for the sulfonium cation M⁺ in formula (1). Of these, the preferred cations are given below, but not limited thereto.

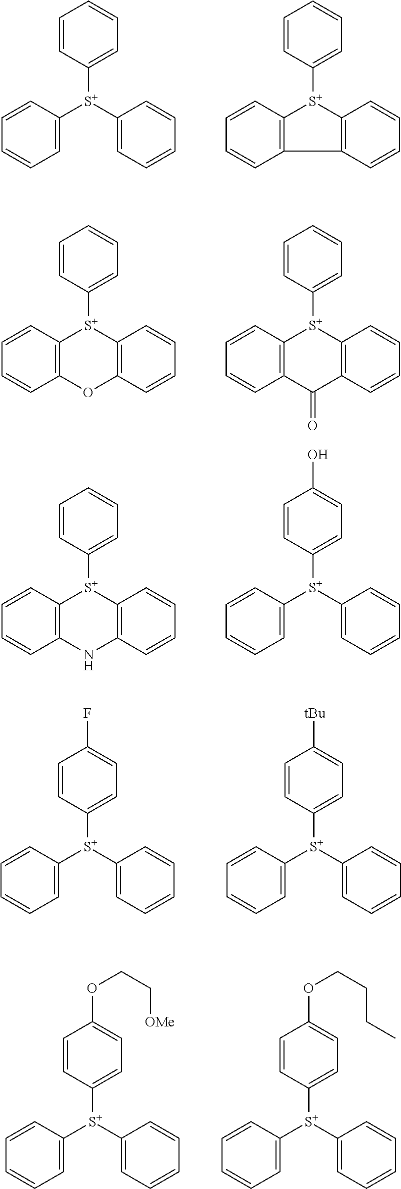

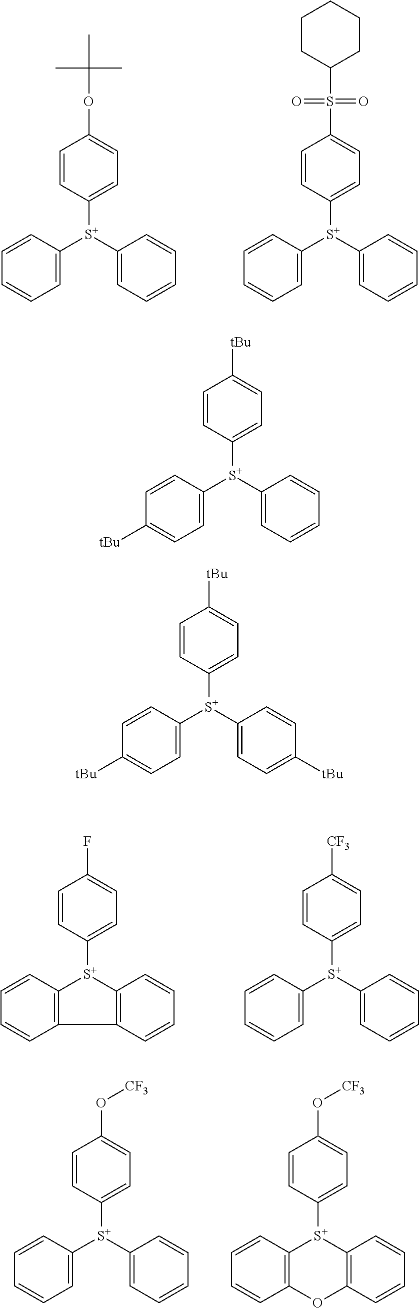

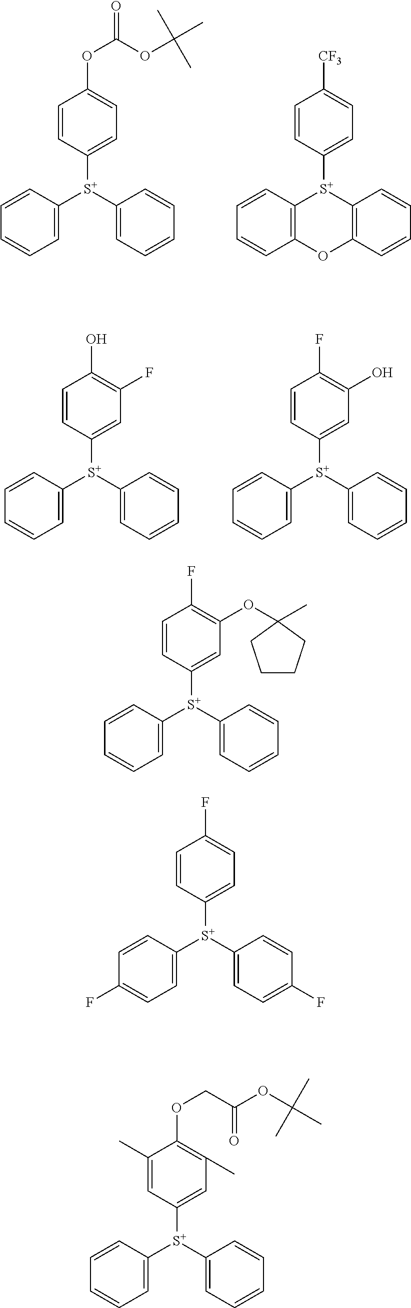

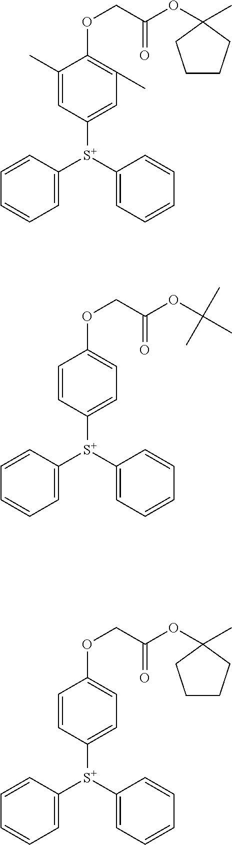

The recurring units (d1) to (d4) have the function of a photoacid generator. On use of a base polymer comprising recurring units (d1) to (d4), a photoacid generator of addition type to be described later may be omitted.

The base polymer may further comprise recurring units (e) containing a hydroxyl group (other than phenolic hydroxyl group), lactone ring, ether bond, ester bond, carbonyl group, cyano group or carboxyl group as another adhesive group.

Examples of the recurring units (e) are given below, but not limited thereto. Herein R^{A} is as defined above.

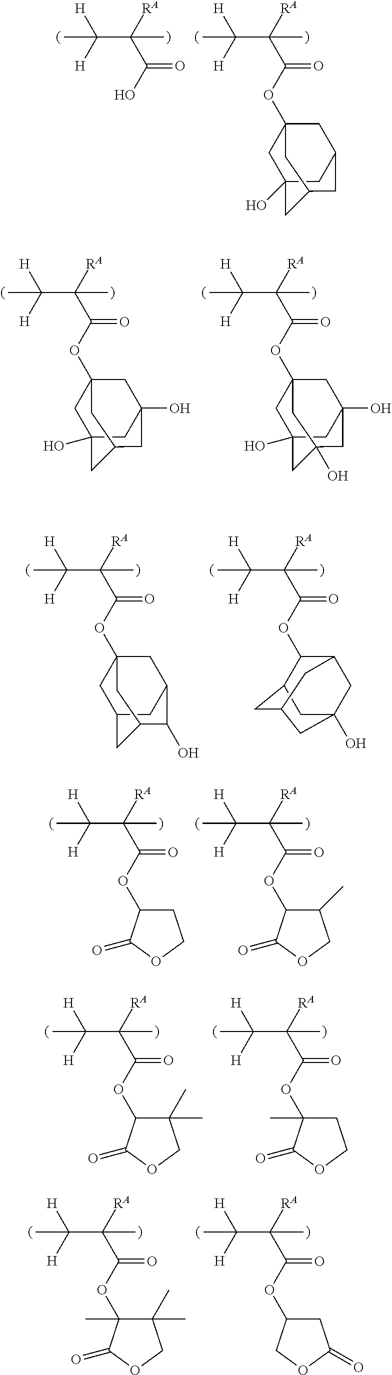

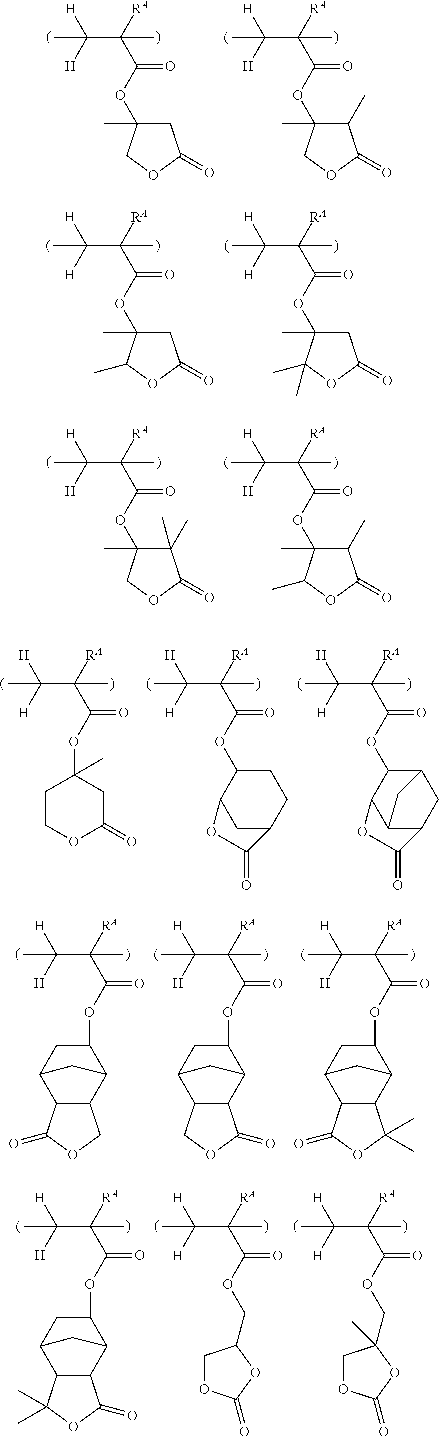

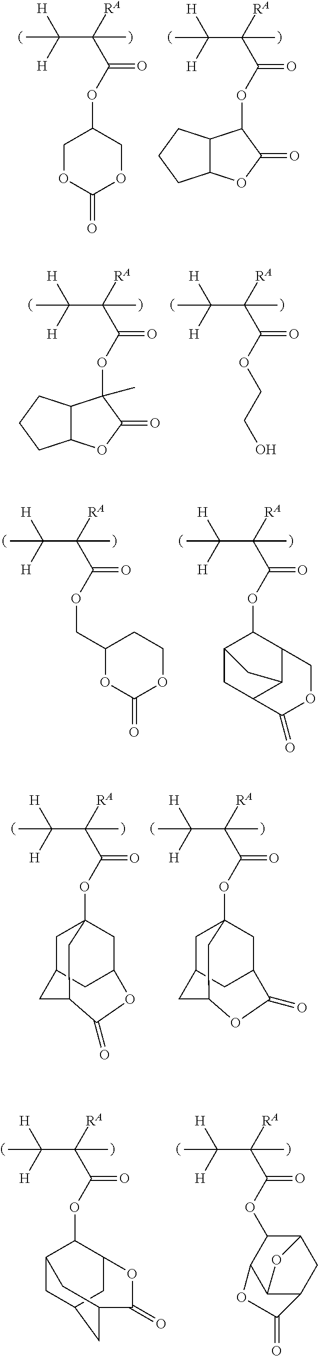

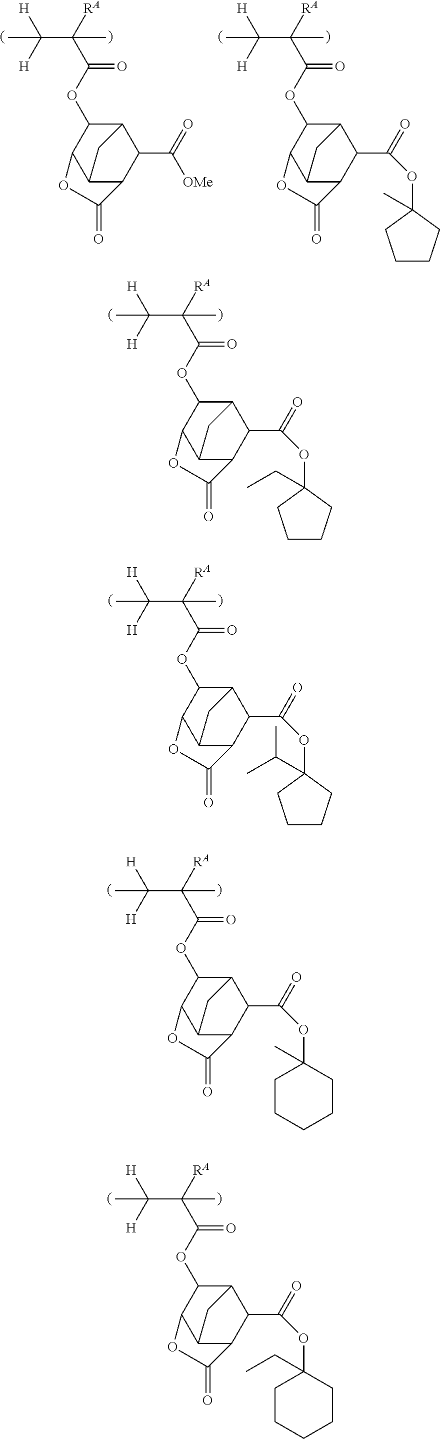

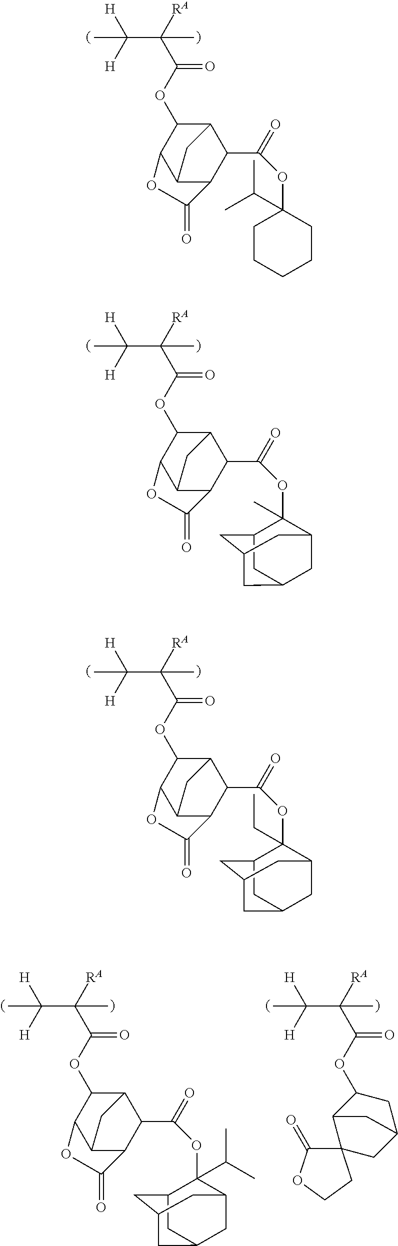

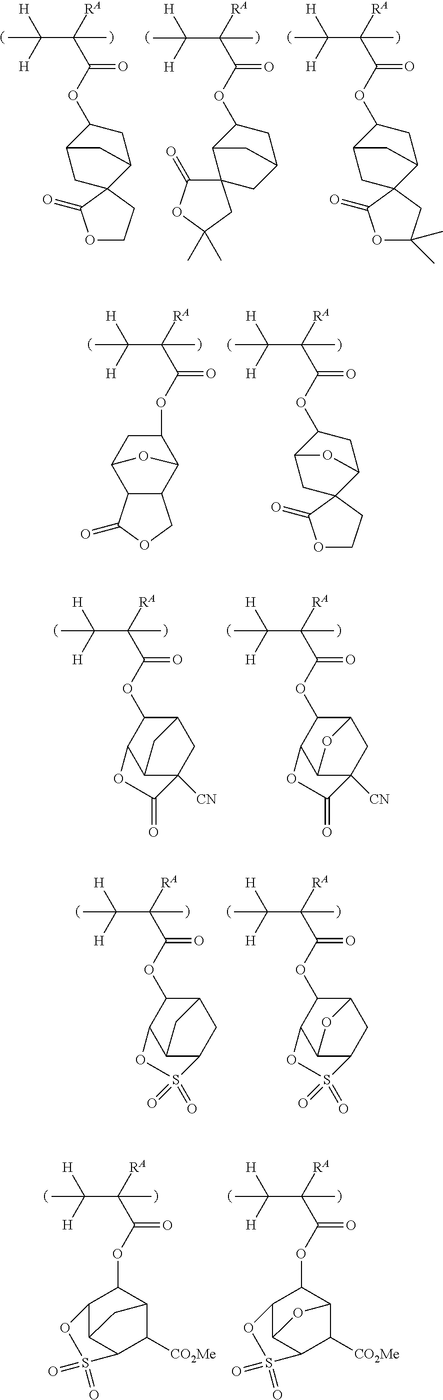

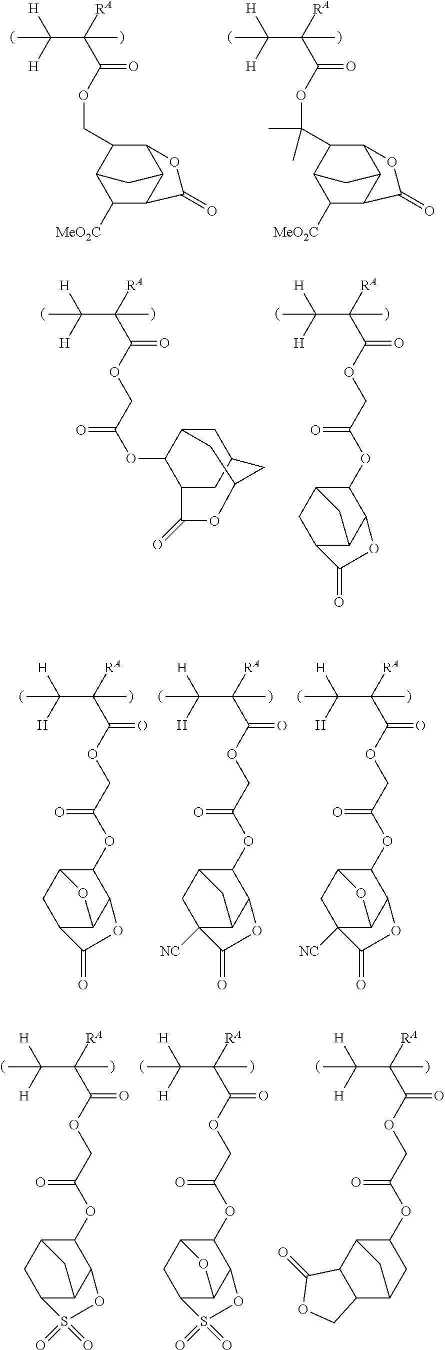

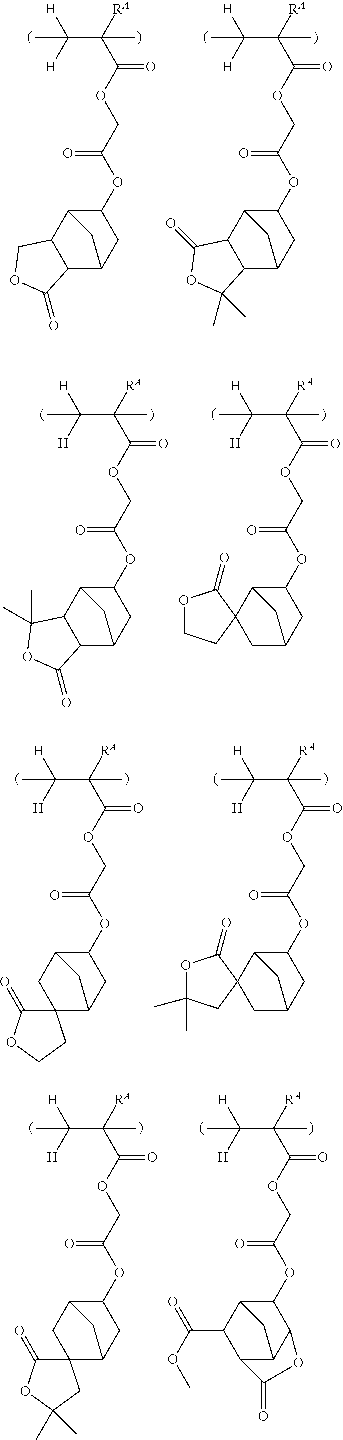

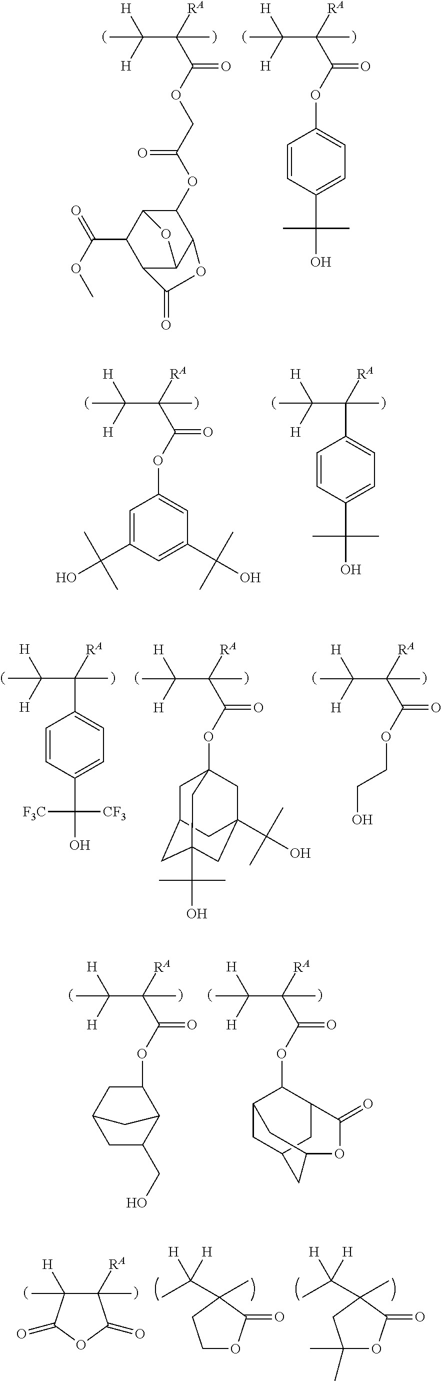

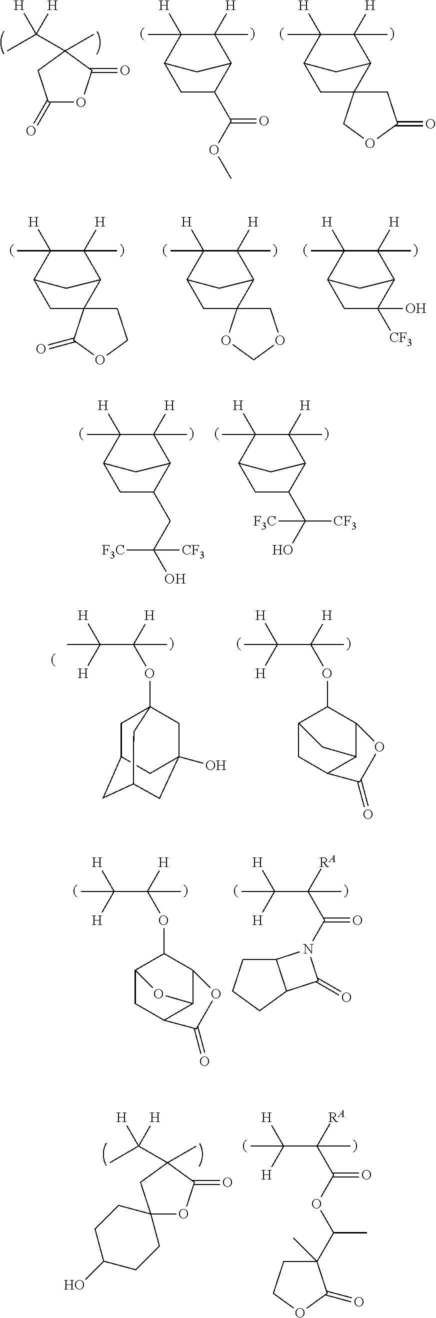

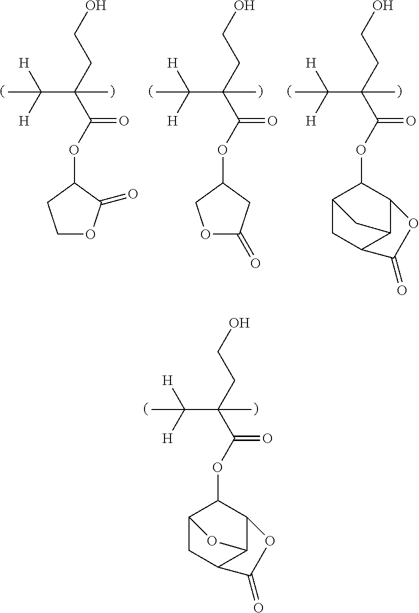

In addition to the foregoing examples, examples of the recurring units (e) include those described in JP-A 2014-225005, paragraphs [0045]-[0053].

Of the foregoing, units having a hydroxyl group or lactone ring are preferred as the recurring unit (e), with preferred examples being shown below.

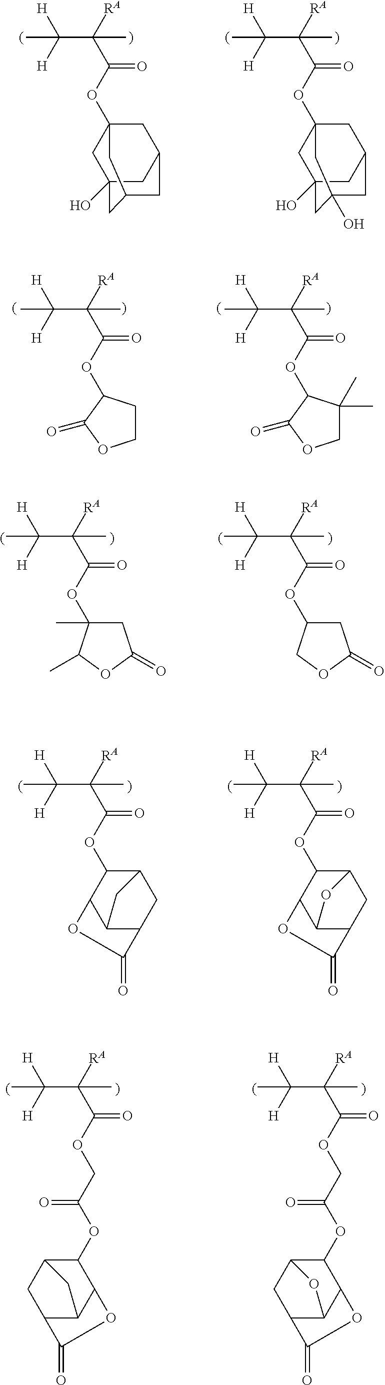

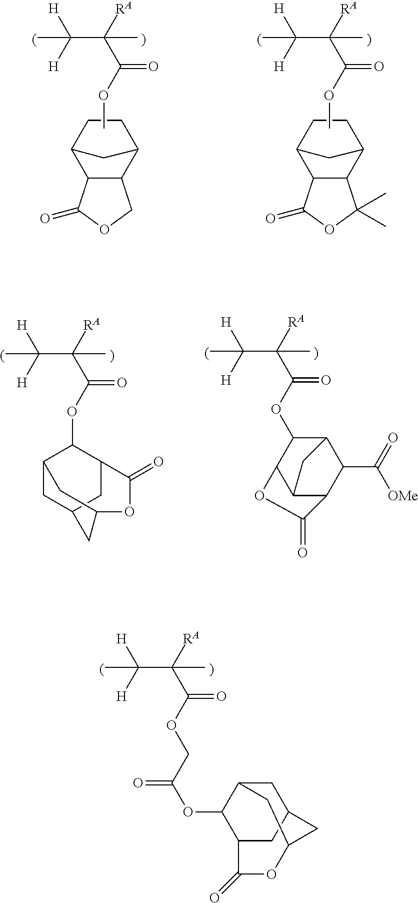

The base polymer may further comprise recurring units of the structure having a hydroxyl group protected with an acid labile group. The recurring unit of the structure having a hydroxyl group protected with an acid labile group is not particularly limited as long as the unit has at least one protected hydroxyl structure wherein a hydroxyl group is resumed as a result of decomposition of the protective group under the action of acid. Such recurring units are described in JP-A 2014-225005, paragraphs [0055]-[0065] and JP-A 2015-214634, paragraphs [0110]-[0115].

The base polymer may further comprise other recurring units. Typical of the other recurring units are recurring units having an oxirane or oxetane ring. A polymer comprising recurring units having an oxirane or oxetane ring is crosslinked in exposed regions, leading to improvements in retention and etching resistance of a resist film in exposed regions.

The base polymer may further comprise still other recurring units, for example, units derived from substituted acrylates such as methyl crotonate, dimethyl maleate, and dimethyl itaconate, unsaturated carboxylic acids such as maleic acid, fumaric acid, and itaconic acid, cyclic olefins such as norbornene, norbornene derivatives, tetracyclo[6.2.1.1^{3,6}.0^{2,7}]dodecene derivatives, unsaturated acid anhydrides such as to itaconic anhydride, vinyl aromatics such as styrene, tert-butoxystyrene, vinylnaphthalene, acetoxystyrene, and acenaphthylene, and other monomers.

The base polymer should preferably have a Mw of 1,000 to 500,000, more preferably 3,000 to 100,000, and even more preferably 4,000 to 20,000. A Mw within the range eliminates an extreme drop of etching resistance and provides satisfactory resolution due to a difference in dissolution rate before and after exposure. As used herein, Mw is measured versus polystyrene standards by GPC. Also preferably the polymer has a dispersity (Mw/Mn) of 1.20 to 2.50, more preferably 1.30 to 2.00.

The polymer may be synthesized by any method, for example, by using one or more monomers corresponding to the desired recurring units in an organic solvent, adding a radical polymerization initiator, and heating for polymerization. For the polymerization method, reference should be made to U.S. Pat. No. 9,256,127 (JP-A 2015-214634, paragraphs [0134]-[0137]. The acid labile group that has been incorporated in the monomer may be kept as such, or polymerization may be followed by protection or partial protection.

While the base polymer comprises recurring units derived from monomers, the molar fractions of respective units preferably fall in the following range (mol %), but are not limited thereto:
(I) 10 to 70 mol %, more preferably 20 to 65 mol %, even more preferably 30 to 60 mol % of recurring units of at least one type selected from recurring units (a) and (b),
(II) 0 to 90 mol %, more preferably 15 to 80 mol %, even more preferably 30 to 60 mol % of recurring units (c) of at least one type, and optionally,
(III) 0 to 30 mol %, more preferably 0 to 20 mol %, and even more preferably 0 to 15 mol % of recurring units of at least one type selected from recurring units (d1) to (d4), and optionally,
(IV) 0 to 80 mol %, more preferably 0 to 70 mol %, and even more preferably 0 to 50 mol % of recurring units of at least one type selected from recurring units (e) and other recurring units.

The base polymer (A) may be used alone or in a combination of two or more polymers which are different in compositional ratio, Mw and/or Mw/Mn. In addition to the polymer, a hydrogenated product of ring-opening metathesis polymerization (ROMP) polymer may to be used. The hydrogenated ROMP polymer is as described in JP-A 2003-066612.

### (B) Photoacid Generator

The resist composition should comprise (B) a photoacid generator, which is sometimes referred to as PAG of addition type, when the base polymer does not contain any of recurring units (d1) to (d4). It is noted that a PAG of addition type may be added even when the base polymer contains recurring units of at least one type selected from recurring units (d1) to (d4).

The PAG of addition type may be any compound capable of generating an acid upon exposure to high-energy radiation. Suitable PAGs include sulfonium salts, iodonium salts, sulfonyldiazomethanes, N-sulfonyloxydicarboxyimides, O-arylsulfonyloximes, and O-alkylsulfonyloximes, which may be used alone or in admixture. Suitable examples are described in JP-A 2007-145797, paragraphs [0102]-[0113], JP-A 2008-111103, paragraphs [0122]-[0142], JP-A 2014-001259, paragraphs [0081]-[0092], JP-A 2012-041320, JP-A 2012-153644, JP-A 2012-106986, and JP-A 2016-018007. The PAGs capable of generating partially fluorinated sulfonic acids described in the foregoing patent documents are preferably used in a resist composition because the strength and diffusion length of the generated acid are appropriate in the ArF lithography.

Preferred as the PAG (B) are sulfonium salts having the formula (5A) and iodonium salts having the formula (5B).

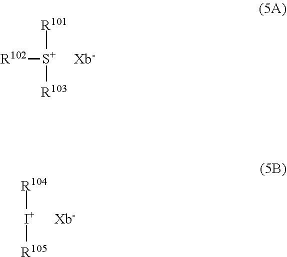

In formulae (5A) and (5B), R¹⁰¹, R¹⁰², R¹⁰³, R¹⁰⁴ and R¹⁰⁵ are each independently a C₁-C₂₀ hydrocarbyl group which may contain a heteroatom. Examples of the hydrocarbyl group are as exemplified above for R³¹ to R⁴¹ in formulae (d1) to (d4). Any two of R¹⁰¹, R¹⁰² and R¹⁰³ may bond together to form a ring with the sulfur atom to which they are attached, and R¹⁰⁴ and R¹⁰⁵ may bond together to form a ring with the iodine atom to which they are attached. Examples of the ring include those exemplified above for the ring that any two of R^{M1}, R^{M2} and R^{M3}, taken together, form with the sulfur atom to which they are attached, in formula (M-1), and those exemplified above for the ring that R^{M4} and R^{M5}, taken together, form with the iodine atom to which they are attached, in formula (M-2). R¹⁰¹ to R¹⁰⁵ are preferably of a structure containing a phenyl group which is bonded to S⁺ or I⁺ in the formula.

The sulfonium cation of the sulfonium salt having formula (5A) is described in JP-A 2014-001259, paragraphs [0082]-[0085]. Exemplary sulfonium cations include those described in JP-A 2007-145797, paragraphs [0027]-[0033], JP-A 2010-113209, paragraph [0059], JP-A 2012-041320, JP-A 2012-153644, and JP-A 2012-106986, as well as those exemplified above for the sulfonium cation M⁺ in formula (1).

Preferred examples of the cation of the sulfonium salt having formula (5A) are given below, but not limited thereto.

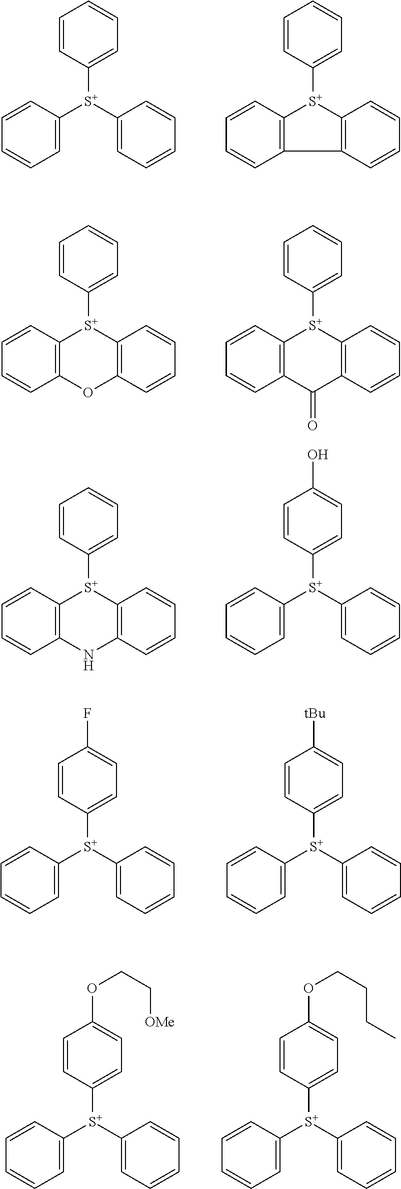

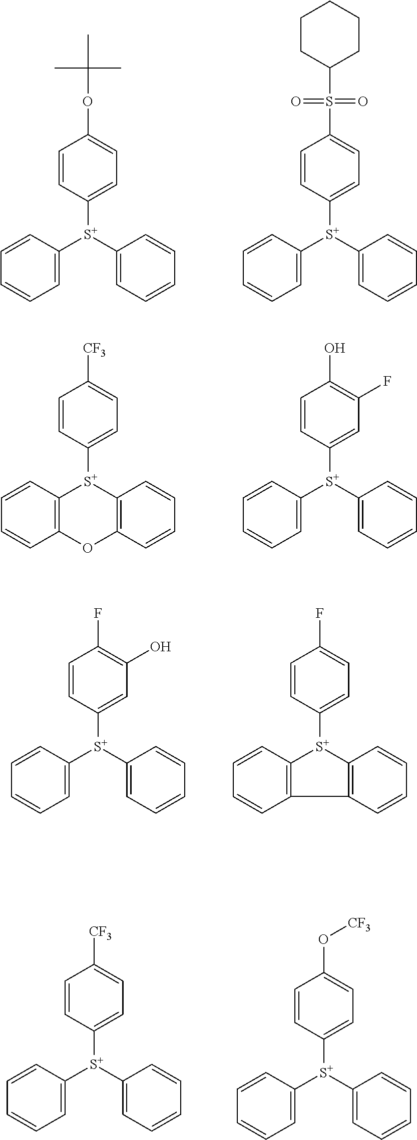

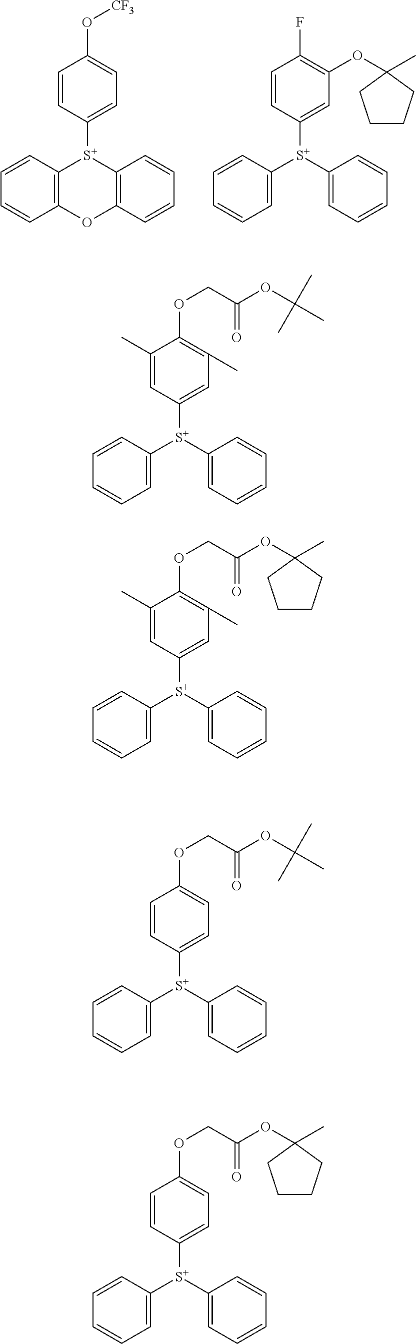 [SMILES: FC(F)(F)Oc1ccc([S+]2c3ccccc3Oc3ccccc32)cc1 ; CC1(Oc2cc([S+](c3ccccc3)c3ccccc3)ccc2F)CCCC1 ; Cc1cc([S+](c2ccccc2)c2ccccc2)cc(C)c1OCC(=O)OC(C)(C)C ; Cc1cc([S+](c2ccccc2)c2ccccc2)cc(C)c1OCC(=O)OC1(C)CCCC1 ; CC(C)(C)OC(=O)COc1ccc([S+](c2ccccc2)c2ccccc2)cc1 ; CC1(OC(=O)COc2ccc([S+](c3ccccc3)c3ccccc3)cc2)CCCC1]

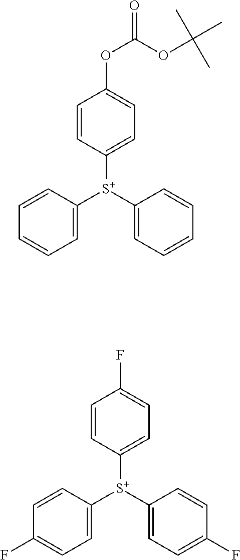 [SMILES: CC(C)(C)OC(=O)Oc1ccc([S+](c2ccccc2)c2ccccc2)cc1 ; Fc1ccc([S+](c2ccc(F)cc2)c2ccc(F)cc2)cc1]

Specific examples of the cation of the sulfonium salt having formula (5A) include triphenylsulfonium, S-phenyldibenzothiophenium, (4-tert-butylphenyl)diphenylsulfonium, (4-fluorophenyl)diphenylsulfonium, and (4-hydroxyphenyl)diphenylsulfonium cations.

Examples of the cation of the iodonium salt having formula (5B) include those exemplified above for the iodonium cation M⁺ in formula (1), with diphenyliodonium and di-tert-butylphenyliodonium cations being preferred.

In formulae (5A) and (5B), Xb⁻ is an anion having the formula (6A) or (6B).

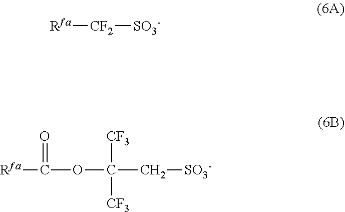

In formulae (6A) and (6B), R^{fa} is fluorine, a C₁-C₄ perfluoroalkyl group, or a C₁-C₄₀ hydrocarbyl group which may contain a heteroatom, in which —CH₂— may be replaced by —O— or —C(═O)—.

Preferred examples of the anion having formula (6A) include trifluoromethanesulfonate and nonafluorobutanesulfonate anions, and anions having the formula (6A′).

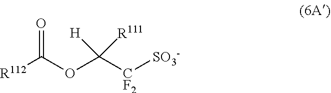

In formula (6A′), R¹¹¹ is hydrogen or trifluoromethyl, preferably trifluoromethyl.

R¹¹² is a C₁-C₃₅ hydrocarbyl group. The hydrocarbyl group may be saturated or unsaturated and straight, branched or cyclic. Examples include alkyl groups such as methyl, ethyl, propyl, isopropyl, n-butyl, sec-butyl, tert-butyl, n-pentyl, n-hexyl, n-octyl, 2-ethylhexyl, n-nonyl, n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, adamantylmethyl; alkenyl groups such as vinyl, allyl, propenyl, butenyl, hexenyl; unsaturated alicyclic hydrocarbyl groups such as cyclohexenyl; aryl groups such as phenyl, naphthyl, thienyl, 4-hydroxyphenyl, 4-methoxyphenyl, 3-methoxyphenyl, 2-methoxyphenyl, 4-ethoxyphenyl, 4-tert-butoxyphenyl, 3-tert-butoxyphenyl, 2-methylphenyl, 3-methylphenyl, 4-methylphenyl, 4-ethylphenyl, 4-tert-butylphenyl, 4-n-butylphenyl, 2,4-dimethylphenyl, 2,4,6-triisopropylphenyl, methylnaphthyl, ethylnaphthyl, methoxynaphthyl, ethoxynaphthyl, n-propoxynaphthyl, n-butoxynaphthyl, dimethylnaphthyl, diethylnaphthyl, dimethoxynaphthyl, diethoxynaphthyl; aralkyl groups such as benzyl, 1-phenylethyl, 2-phenylethyl, and combinations thereof. Some or all of the hydrogen atoms in the hydrocarbyl group may be substituted by a moiety containing a heteroatom such as oxygen, sulfur, nitrogen or halogen, and —CH₂— in the hydrocarbyl group may be replaced by —O— or —C(═O)—, so that the group may contain a hydroxyl moiety, cyano moiety, carbonyl moiety, ether bond, ester bond, carbonate bond, lactone ring, carboxylic anhydride or haloalkyl moiety.

The anion having formula (6A′) is described in JP-A 2007-145797, JP-A 2008-106045, JP-A 2009-007327, JP-A 2009-258695, and JP-A 2012-181306. Examples of the anion having formula (6A) include those described in these patent documents and those exemplified above as the anion having formula (d1-1).

In formula (6B), R^{fb} is a C₁-C₄₀ hydrocarbyl group. Some or all of the hydrogen atoms in the hydrocarbyl group may be substituted by a moiety containing a heteroatom such as oxygen, sulfur, nitrogen or halogen, and —CH₂— in the hydrocarbyl group may be replaced by —O— or —C(═O)—. Examples of the hydrocarbyl group R^{fb} are as exemplified above for R¹¹².

The anion having formula (6B) is described in JP-A 2010-215608 and JP-A 2014-133723. Examples of the anion having formula (6B) include those described in these patent documents and those exemplified above as the anion having formula (d1-2). Notably, the compound having the anion of formula (6B) does not have fluorine at the α-position relative to the sulfo group, but two trifluoromethyl groups at the β-position. For this reason, it has a sufficient acidity to sever the acid labile groups in the base polymer. Thus the compound is an effective PAG.

Preferred examples of the anion Xb⁻ are shown below, but not limited thereto. Herein R^{HF} is hydrogen or trifluoromethyl.

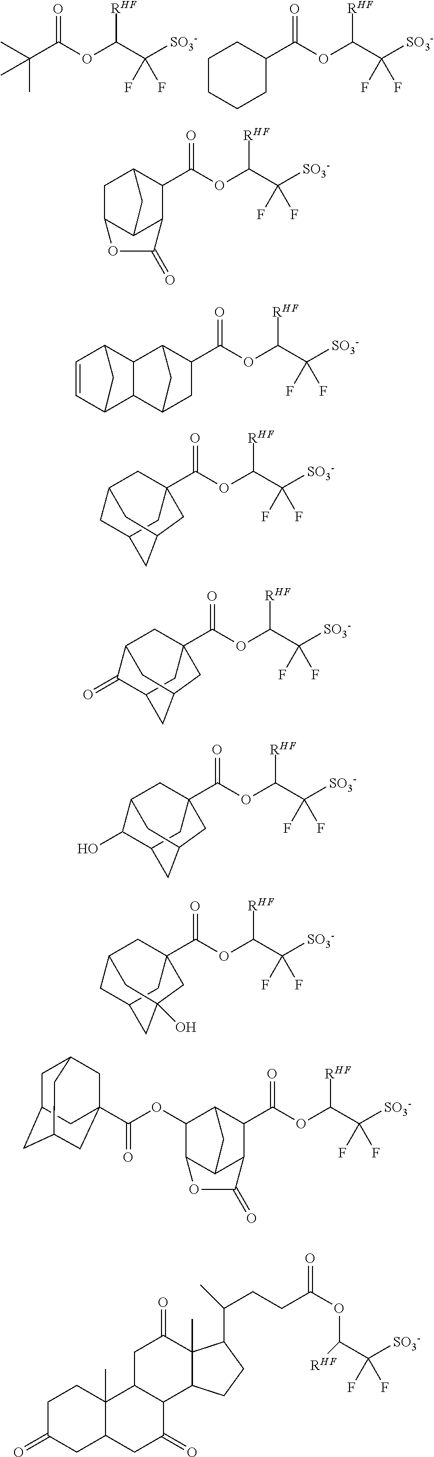

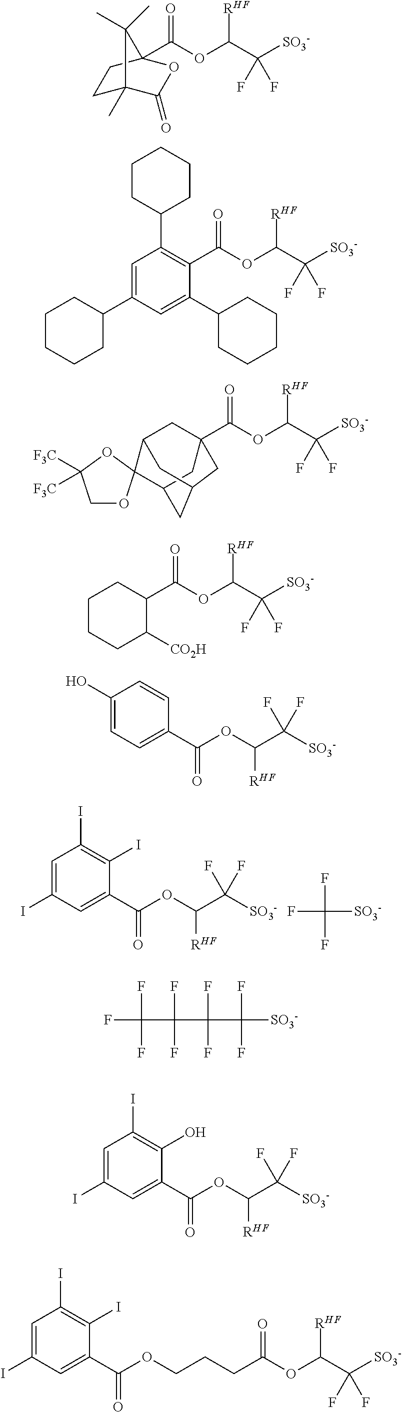

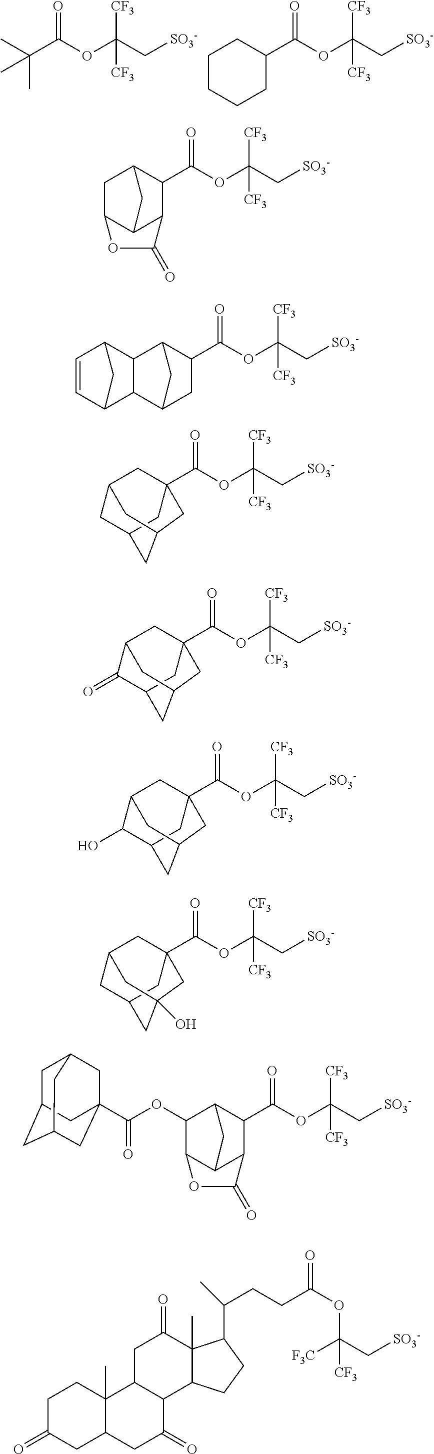

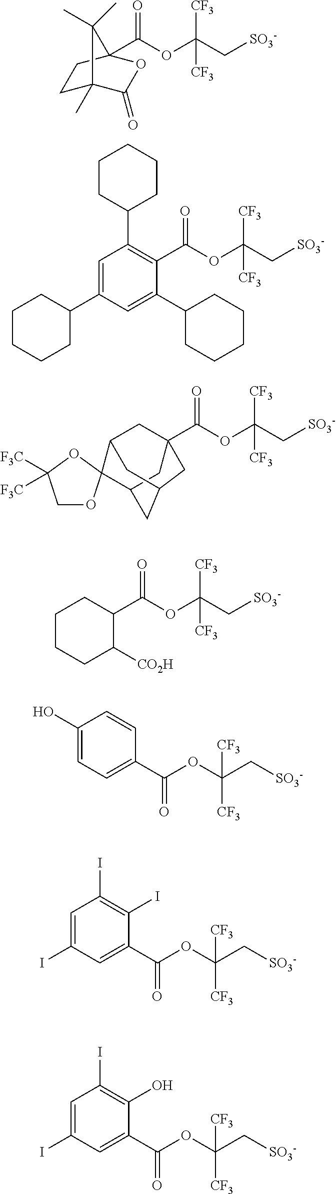

Exemplary structures for the PAG having formula (5A) or (5B) include arbitrary combinations of cations with anions, both as exemplified above, but are not limited thereto.

Another preferred example of the PAG (B) is a compound having the formula (7).

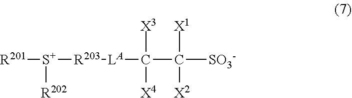

In formula (7), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The hydrocarbyl groups R²⁰¹ and R²⁰² may be saturated or unsaturated and straight, to branched or cyclic. Examples thereof are as exemplified above for R¹¹².

The hydrocarbylene group R²⁰³ may be saturated or unsaturated and straight, branched or cyclic. Examples thereof include alkanediyl groups such as methylene, ethylene, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, and tetradecane-1,14-diyl; cyclic saturated hydrocarbylene groups such as cyclopentanediyl, cyclohexanediyl, norbornanediyl, and adamantanediyl; arylene groups such as phenylene, methylphenylene, ethylphenylene, n-propylphenylene, isopropylphenylene, n-butylphenylene, isobutylphenylene, sec-butylphenylene, tert-butylphenylene, dimethylphenylene, diethylphenylene, naphthylene, methylnaphthylene, ethylnaphthylene, n-propylnaphthylene, isopropylnaphthylene, n-butylnaphthylene, isobutylnaphthylene, sec-butylnaphthylene, tert-butylnaphthylene, dimethylnaphthylene, diethylnaphthylene, and combinations thereof. In the hydrocarbylene group, some hydrogen may be substituted by a moiety containing a heteroatom such as oxygen, sulfur, nitrogen or halogen, and a moiety containing a heteroatom such as oxygen, sulfur or nitrogen may intervene in a carbon-carbon bond, so that the group may contain a hydroxyl, cyano, carbonyl, ether bond, ester bond, sulfonate bond, carbonate bond, lactone ring, sultone ring, carboxylic anhydride or haloalkyl moiety.

In formula (7), L^{A} is a single bond, ether bond, ester bond, or a C₁-C₂₀ hydrocarbylene group which may contain a heteroatom, in which —CH₂— may be replaced by —O— or —C(═O)—. The constituent —CH₂— in the hydrocarbyl group may be one bonding to the carbon atom and/or R²⁰³ in formula (7). The hydrocarbylene group L^{A} may be saturated or unsaturated and straight, branched or cyclic. Examples thereof are as exemplified above for R²⁰³.

In formula (7), X¹, X², X³ and X⁴ are each independently hydrogen, fluorine or trifluoromethyl, with at least one thereof being fluorine or trifluoromethyl.

Of the compounds having formula (7), those having formula (7′) are more preferred.

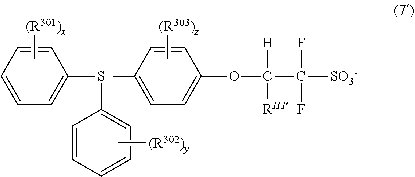

In formula (7′), R^{HF} is hydrogen or trifluoromethyl, preferably trifluoromethyl. R³⁰¹, R³⁰² and R³⁰³ are each independently a C₁-C₂₀ hydrocarbyl group in which some or all of the hydrogen atoms may be substituted by a moiety containing a heteroatom such as oxygen, sulfur, nitrogen or halogen, and in which —CH₂— may be replaced by —O— or —C(═O)—. The constituent —CH₂— in the hydrocarbyl group may be one bonding to a carbon atom on the benzene ring in formula (7′). The hydrocarbyl groups R³⁰¹, R³⁰² and R³⁰³ may be saturated or unsaturated and straight, branched or cyclic. Examples thereof are as exemplified above for R¹¹². The subscripts x and y are each independently an integer of 0 to 5, and z is an integer of 0 to 4.

The PAG having formula (7) or (7′) is described in JP-A 2011-016746. Examples thereof include those exemplified for the sulfonium salt in the same patent document and those exemplified for the sulfonium salt in JP-A 2015-214634, paragraphs [0149]-[0150].

Specific examples of the PAG having formula (7) are given below, but not limited thereto. Herein R^{HF} is as defined above.

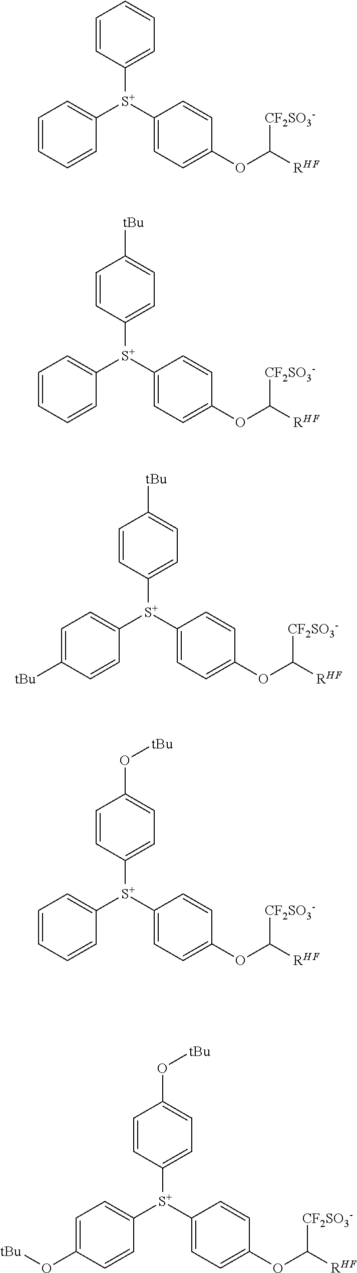

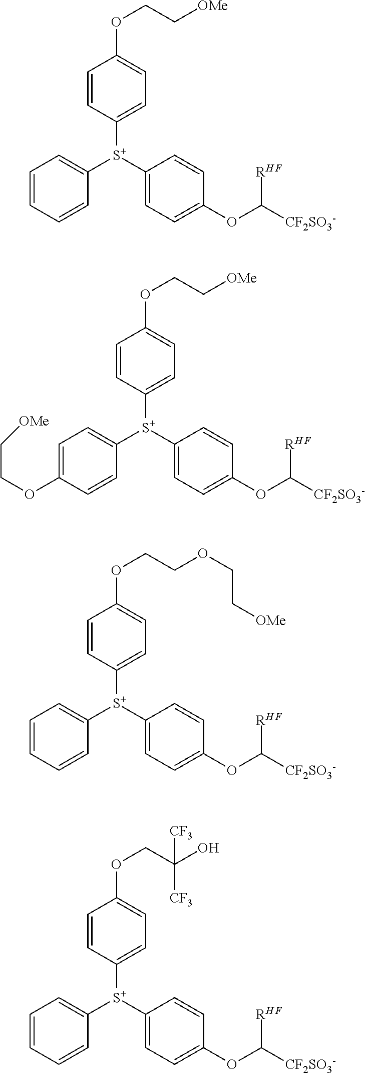

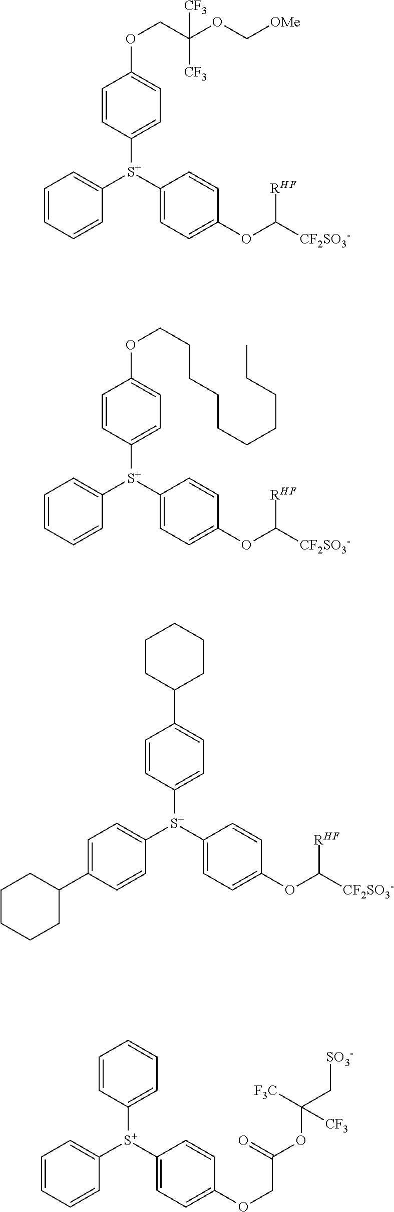

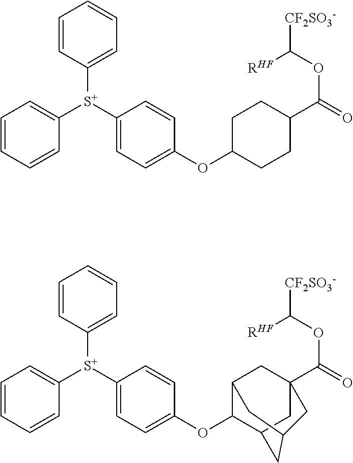

The PAG (B) is preferably added in an amount of 1 to 30 parts by weight, more preferably 2 to 25 parts by weight, even more preferably 4 to 20 parts by weight per 100 parts by weight of the base polymer (A). The PAG in the range eliminates the problems of degradation of resolution and formation of foreign matter after development or during stripping. The PAG may be used alone or in admixture.

### (C) Acid Diffusion Inhibitor

The resist composition further comprises (C) an acid diffusion inhibitor. Component (C) should contain (C-1) the onium salt compound having formula (1) as an essential component and may contain (C-2) an acid diffusion inhibitor other than the onium salt compound having formula (1). As used herein, the “acid diffusion inhibitor” refers to a compound capable of holding down the diffusion rate when the acid generated by the PAG diffuses in the resist film.

The acid diffusion inhibitor (C-2) is typically selected from amine compounds and onium salts of weak acids such as α-non-fluorinated sulfonic acids and carboxylic acids.

Examples of the amine compound include primary, secondary, and tertiary amine compounds, specifically amine compounds having a hydroxyl group, ether bond, ester bond, lactone ring, cyano group or sulfonate bond. Primary and secondary amine compounds protected with a carbamate group are also included. Such protected amine compounds are effective when the resist composition contains a base labile component. Suitable acid diffusion inhibitors include the compounds described in JP-A 2008-111103, paragraphs [0146]-[0164], and JP 3790649 as well as the following compounds, but are not limited thereto.

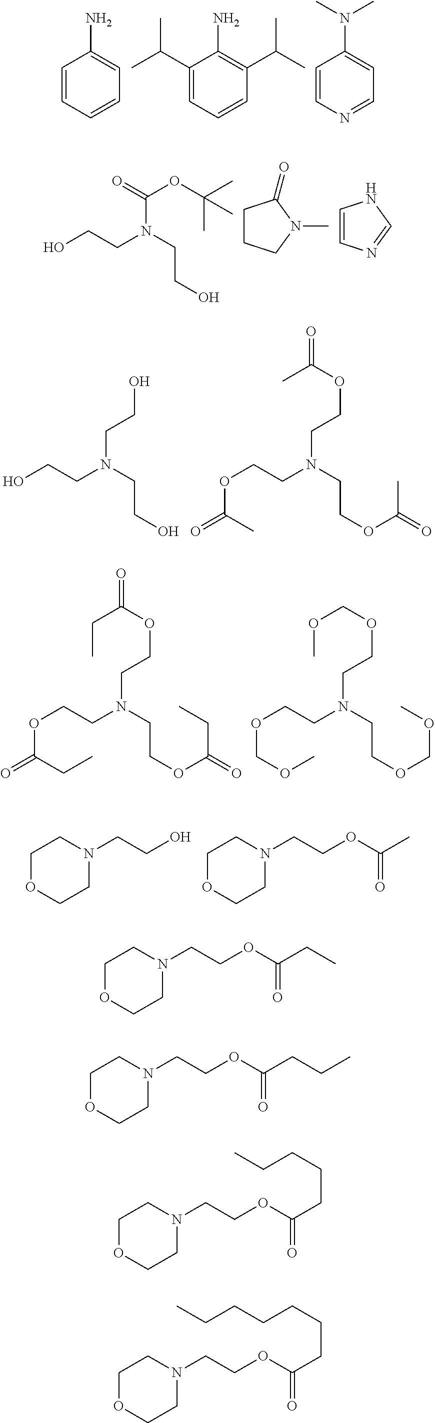

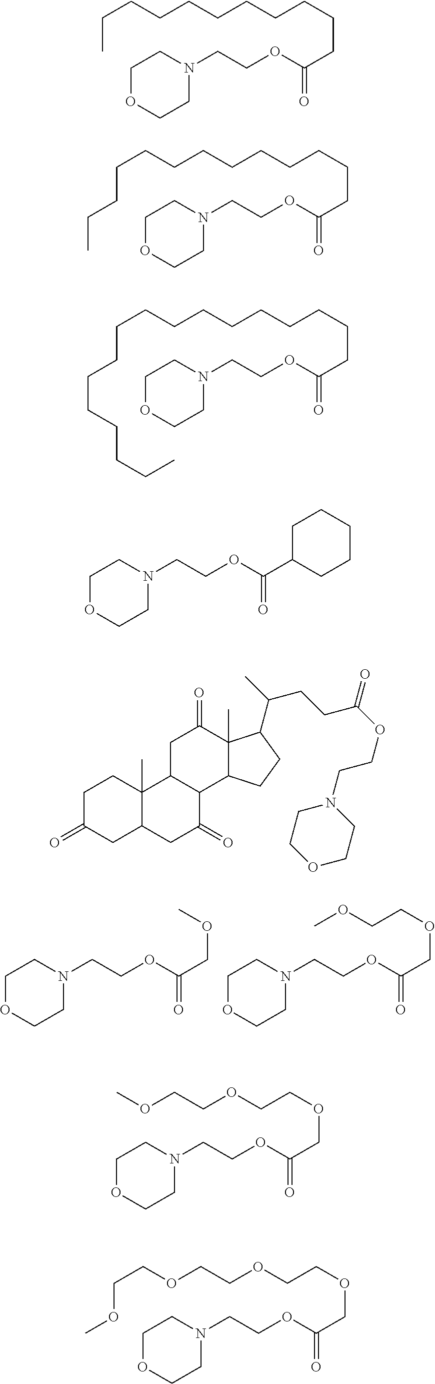

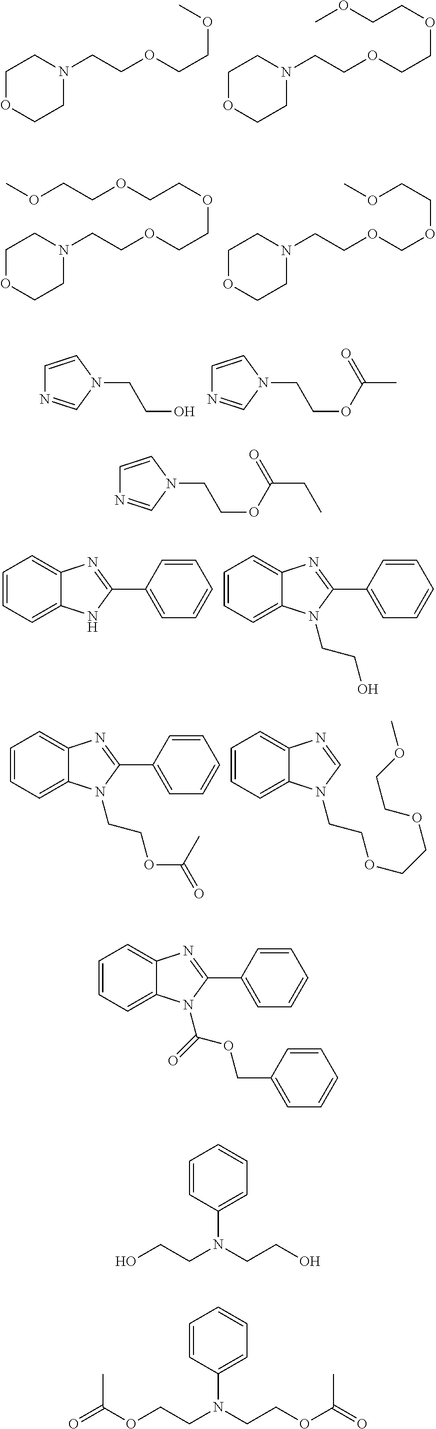

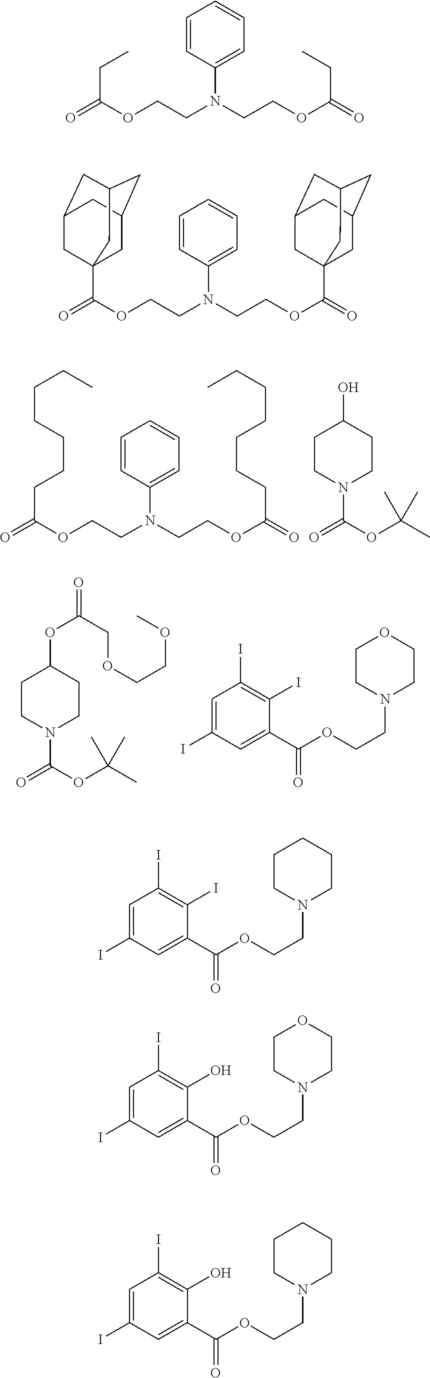

Suitable onium salts of α-non-fluorinated sulfonic acids and carboxylic acids include onium salt compounds having the formulae (8A) and (8B).

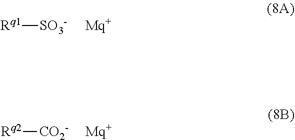

In formula (8A), R^{q1} is hydrogen, methoxy, or a C₁-C₄₀ hydrocarbyl group which may contain a heteroatom, exclusive of the group wherein hydrogen bonded to the carbon atom at α-position relative to the sulfo group is substituted by fluorine or fluoroalkyl.

In formula (8B), R^{q2} is hydrogen, hydroxyl or a C₁-C₄₀ hydrocarbyl group which may contain a heteroatom.

In formulae (8A) and (8B), Mq⁺ is an onium cation, which is preferably selected from cations having the formulae (9A), (9B) and (9C).

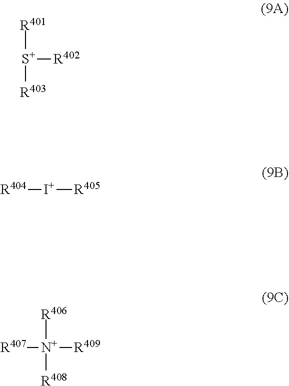

In formulae (9A) to (9C), R⁴⁰¹ to R⁴⁰⁹ are each independently a C₁-C₄₀ hydrocarbyl group which may contain a heteroatom. A pair of R⁴⁰¹ and R⁴⁰², R⁴⁰⁴ and R⁴⁰⁵, or R⁴⁰⁶ and R⁴⁰⁷ may bond together to form a ring with the sulfur, iodine or nitrogen atom to which they are attached.

The optionally heteroatom-containing C₁-C₄₀ hydrocarbyl group, represented by R^{q1}, may be saturated or unsaturated and straight, branched or cyclic. Examples thereof include alkyl groups such as methyl, ethyl, propyl, isopropyl, n-butyl, sec-butyl, tert-butyl, tert-pentyl, n-pentyl, n-hexyl, n-octyl, 2-ethylhexyl, n-nonyl, and n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, and adamantylmethyl; alkenyl groups such as vinyl, allyl, propenyl, butenyl, and hexenyl; cyclic unsaturated hydrocarbyl groups such as cyclohexenyl; aryl groups such as phenyl and naphthyl; heteroaryl groups such as thienyl; hydroxyphenyl groups such as 4-hydroxyphenyl; alkoxyphenyl groups such as 4-methoxyphenyl, 3-methoxyphenyl, 2-methoxyphenyl, 4-ethoxyphenyl, 4-tert-butoxyphenyl, and 3-tert-butoxyphenyl; alkylphenyl groups such as 2-methylphenyl, 3-methylphenyl, 4-methylphenyl, 4-ethylphenyl, 4-tert-butylphenyl, 4-n-butylphenyl, 2,4-dimethylphenyl and 2,4,6-triisopropylphenyl; alkylnaphthyl groups such as methylnaphthyl and ethylnaphthyl; alkoxynaphthyl groups such as methoxynaphthyl, ethoxynaphthyl, n-propoxynaphthyl and n-butoxynaphthyl; dialkylnaphthyl groups such as dimethylnaphthyl and diethylnaphthyl; dialkoxynaphthyl groups such as dimethoxynaphthyl and diethoxynaphthyl; aralkyl groups such as benzyl, 1-phenylethyl and 2-phenylethyl; aryloxoalkyl groups, typically 2-aryl-2-oxoethyl groups such as 2-phenyl-2-oxoethyl, 2-(1-naphthyl)-2-oxoethyl, 2-(2-naphthyl)-2-oxoethyl; and combinations thereof. In the hydrocarbyl group, some or all of the hydrogen atoms may be substituted by a moiety containing a heteroatom such as oxygen, sulfur, nitrogen or halogen, and a moiety containing a heteroatom such as oxygen, sulfur or nitrogen may intervene in a carbon-carbon bond, so that the group may contain a hydroxyl moiety, cyano moiety, carbonyl moiety, ether bond, ester bond, sulfonic acid ester bond, carbonate bond, lactone ring, sultone ring, carboxylic anhydride, or haloalkyl moiety.

The optionally heteroatom-containing C₁-C₄₀ hydrocarbyl group, represented by R^{q2}, may be saturated or unsaturated and straight, branched or cyclic. Examples thereof include those exemplified above for R^{q1} and fluorinated alkyl groups such as trifluoromethyl, trifluoroethyl, 2,2,2-trifluoro-1-methyl-1-hydroxyethyl, and 2,2,2-trifluoro-1-(trifluoromethyl)-1-hydroxyethyl and fluorinated aryl groups such as pentafluorophenyl and 4-trifluoromethylphenyl.

The sulfonic acid onium salt having formula (8A) and the carboxylic acid onium salt having formula (8B) are described in JP-A 2008-158339 and JP-A 2010-155824. Examples thereof are as exemplified in these patent documents.

Examples of the anion in the sulfonic acid onium salt having formula (8A) are shown below, but not limited thereto.

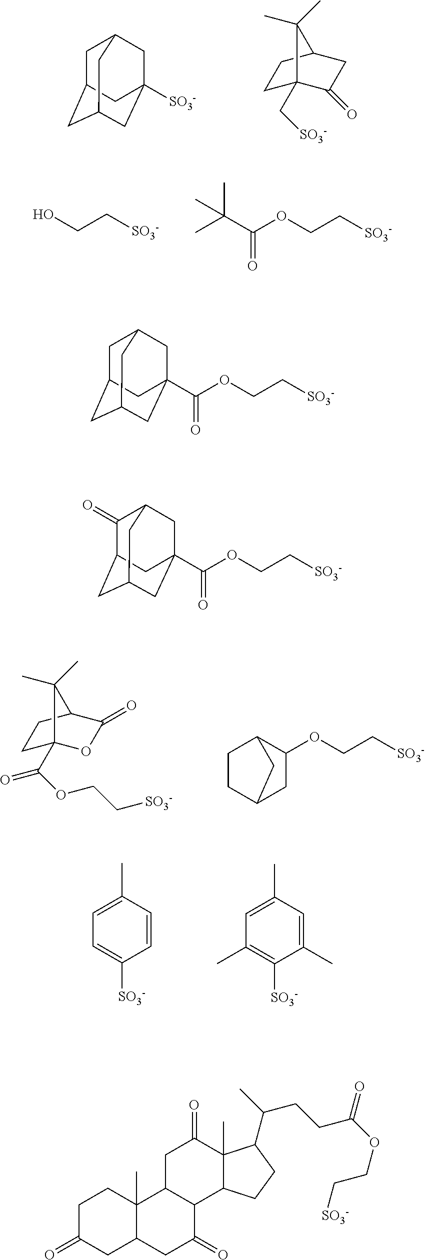

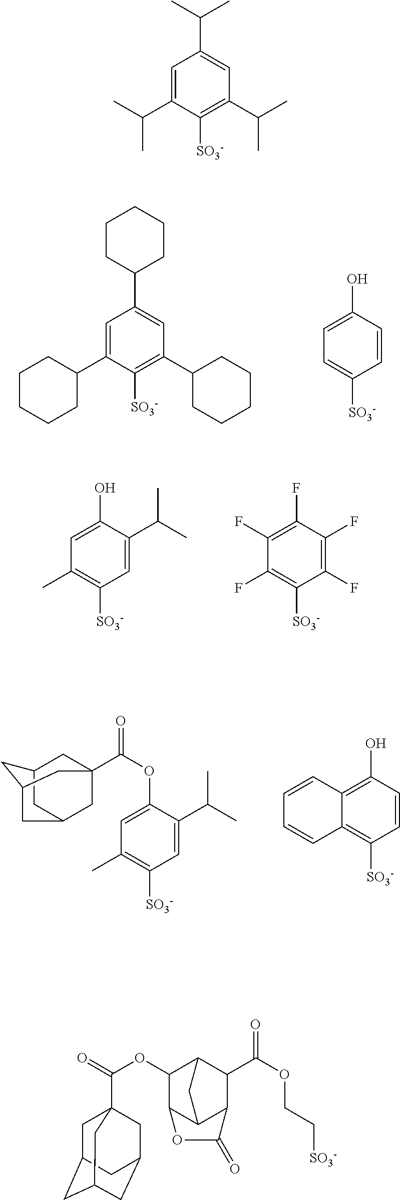

Examples of the anion in the carboxylic acid onium salt having formula (8B) are shown below, but not limited thereto.

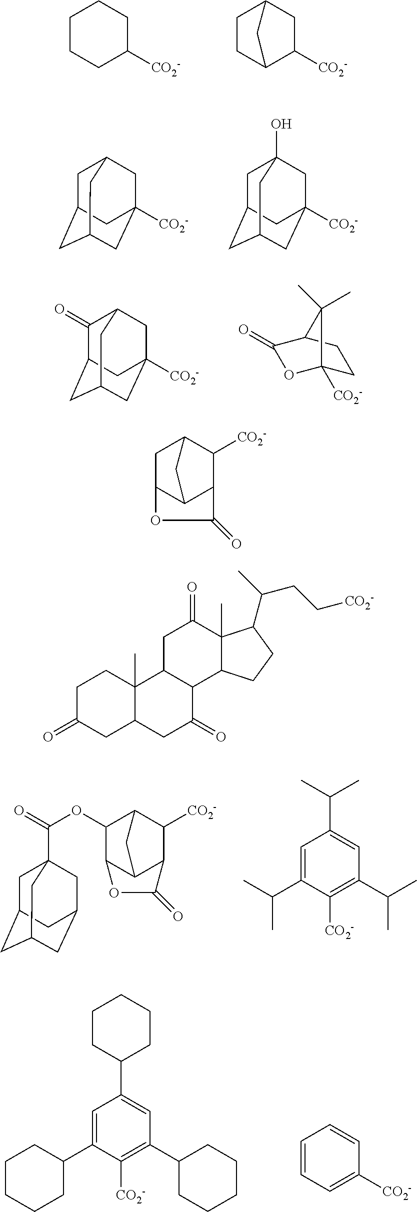

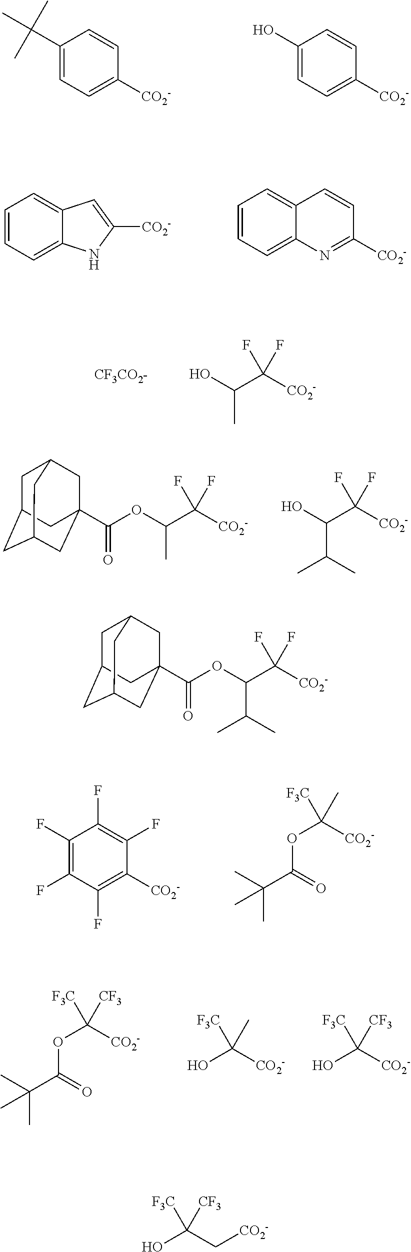

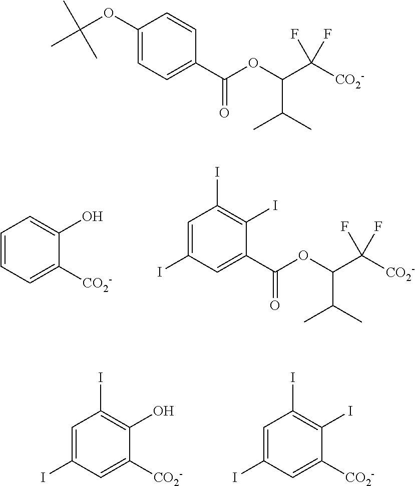

Examples of the cation in formula (9A) and the cation in formula (9B) are as exemplified above for the cation in formula (M-1) and the cation in formula (M-2), respectively, but not limited thereto. Examples of the cation in formula (9C) include tetramethylammonium, tetraethylammonium, tetrabutylammonium, trimethylbenzyl, and trimethylphenyl cations, but are not limited thereto. Inter alia, more preferred cations are shown below.

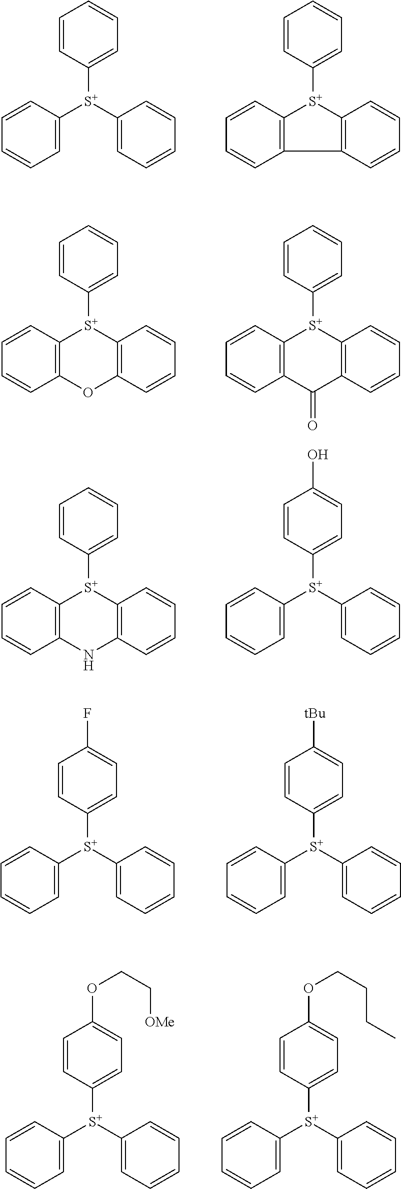

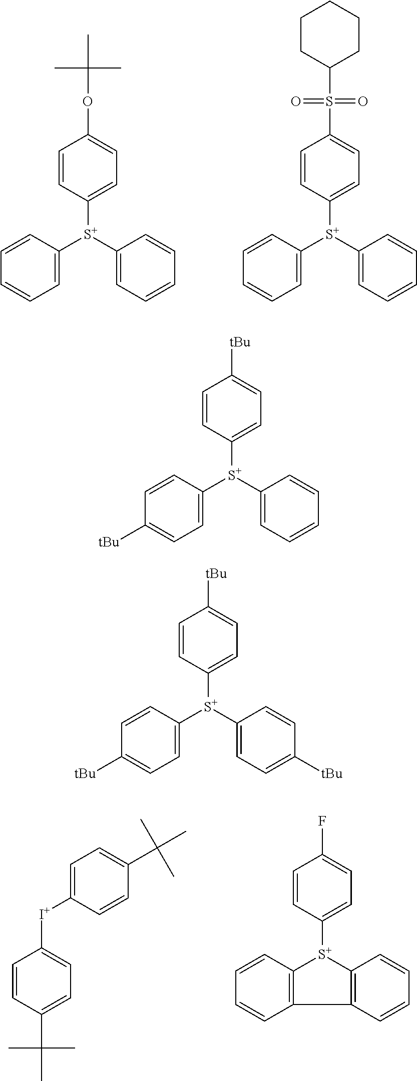 [SMILES: CC(C)(C)Oc1ccc([S+](c2ccccc2)c2ccccc2)cc1 ; O=S(=O)(c1ccc([S+](c2ccccc2)c2ccccc2)cc1)C1CCCCC1 ; CC(C)(C)c1ccc([S+](c2ccccc2)c2ccc(C(C)(C)C)cc2)cc1 ; CC(C)(C)c1ccc([S+](c2ccc(C(C)(C)C)cc2)c2ccc(C(C)(C)C)cc2)cc1 ; CC(C)(C)c1ccc([I+]c2ccc(C(C)(C)C)cc2)cc1 ; Fc1ccc(-[s+]2c3ccccc3c3ccccc32)cc1]

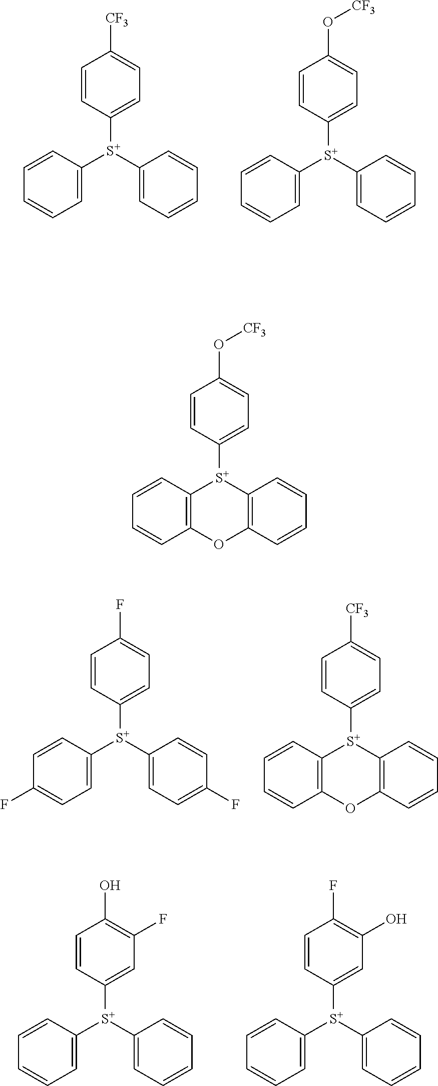

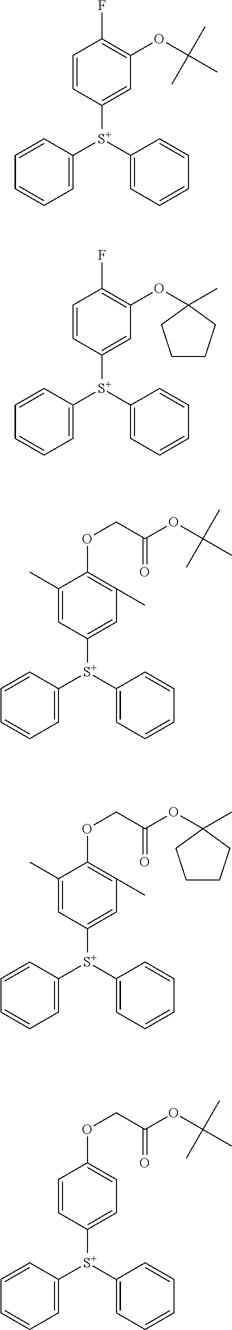 [SMILES: CC(C)(C)Oc1cc([S+](c2ccccc2)c2ccccc2)ccc1F ; CC1(Oc2cc([S+](c3ccccc3)c3ccccc3)ccc2F)CCCC1 ; Cc1cc([S+](c2ccccc2)c2ccccc2)cc(C)c1OCC(=O)OC(C)(C)C ; Cc1cc([S+](c2ccccc2)c2ccccc2)cc(C)c1OCC(=O)OC1(C)CCCC1 ; CC(C)(C)OC(=O)COc1ccc([S+](c2ccccc2)c2ccccc2)cc1]

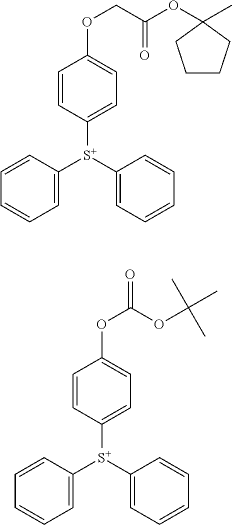 [SMILES: CC1(OC(=O)COc2ccc([S+](c3ccccc3)c3ccccc3)cc2)CCCC1 ; CC(C)(C)OC(=O)Oc1ccc([S+](c2ccccc2)c2ccccc2)cc1]

Examples of the sulfonic acid onium salt having formula (8A) and the carboxylic acid onium salt having formula (8B) include arbitrary combinations of anions with cations, both as exemplified above. These onium salts may be readily synthesized by ion exchange reaction according to any well-known organic chemistry technique. For the ion exchange reaction, reference may be made to JP-A 2007-145797, for example.

The onium salt having formula (8A) or (8B) functions as an acid diffusion inhibitor in the resist composition because the counter anion of the onium salt is a conjugated base of a weak acid. As used herein, the weak acid indicates an acidity insufficient to deprotect an acid labile group from an acid labile group-containing unit in the base polymer. The onium salt having formula (8A) or (8B) functions as an acid diffusion inhibitor when used in combination with an onium salt type PAG having a conjugated base of a strong acid (typically α-fluorinated sulfonic acid) as the counter anion. In a system using a mixture of an onium salt capable of generating a strong acid (e.g., α-fluorinated sulfonic acid) and an onium salt capable of generating a weak acid (e.g., non-fluorinated sulfonic acid or carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

Since the onium salt compound having formula (8A) or (8B) wherein Mq⁺ is a sulfonium cation (9A) or iodonium cation (9B) is photo-decomposable, the quenching ability is reduced and the concentration of strong acid derived from the PAG is increased in the region with high light intensity. Thus the contrast is improved in the exposed region. As a result, a pattern with improved LWR or CDU can be formed.

In case the acid labile group is an acetal group which is quite sensitive to acid, the acid for eliminating the protective group need not necessarily be an α-fluorinated sulfonic acid, imide acid or methide acid. Sometimes, deprotection reaction can take place even with an α-non-fluorinated sulfonic acid. In this case, an amine compound or carboxylic acid onium salt having formula (8B) is preferably used as the acid diffusion inhibitor.

Besides the onium salt, a betaine type compound of weak acid may also be used as the acid diffusion inhibitor. Suitable betaine type compounds are shown below, but not limited thereto.

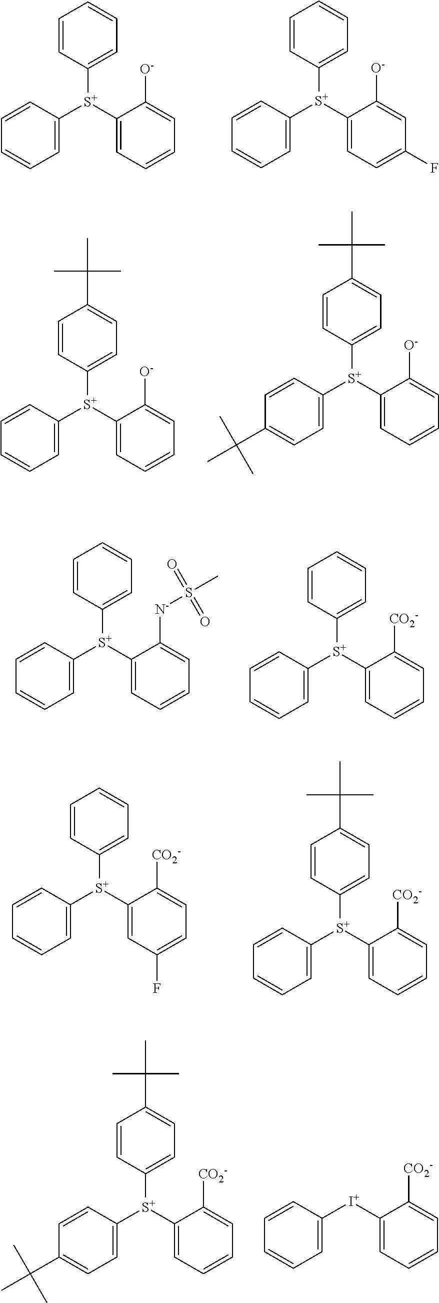

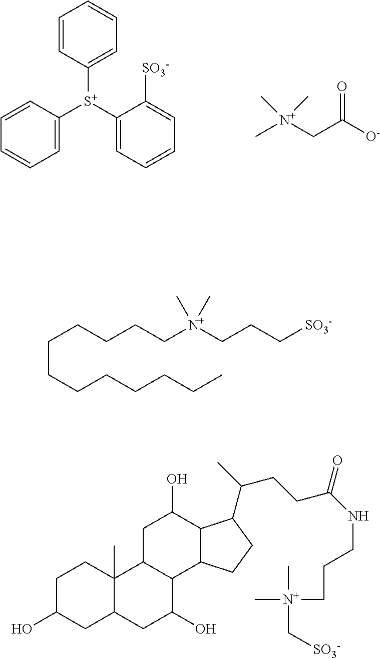

Besides the foregoing compounds, sulfonium or iodonium salts having Cl⁻, Br⁻ or NO₃⁻ as the anion may be used as the acid diffusion inhibitor. Examples include triphenylsulfonium chloride, diphenyliodonium chloride, triphenylsulfonium bromide, and triphenylsulfonium nitrate. Since the conjugate acid corresponding to the anion has a low boiling point, the acid created after quenching of strong acid is readily removed from the resist film during PEB or the like. Due to easy removal of acid from within the resist film, acid diffusion is fully suppressed, resulting in an improvement in contrast.

Also a photo-decomposable onium salt having a nitrogen-containing substituent may be used as the acid diffusion inhibitor. The photo-decomposable onium salt functions as an acid diffusion inhibitor in the unexposed region, but as a so-called photo-degradable base in the exposed region because it loses the acid diffusion inhibitory ability due to neutralization thereof with the acid generated by itself. Using a photo-degradable base, the contrast between exposed and unexposed regions can be further enhanced. With respect to the photo-degradable base, reference may be made to JP-A 2009-109595, 2012-046501, and 2013-209360, for example.

Examples of the anion in the photo-degradable onium salt are shown below, but not limited thereto. Herein R^{HF} is hydrogen or trifluoromethyl.

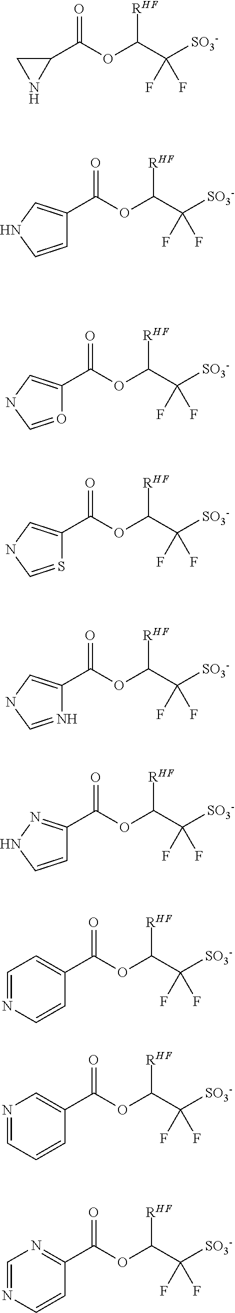

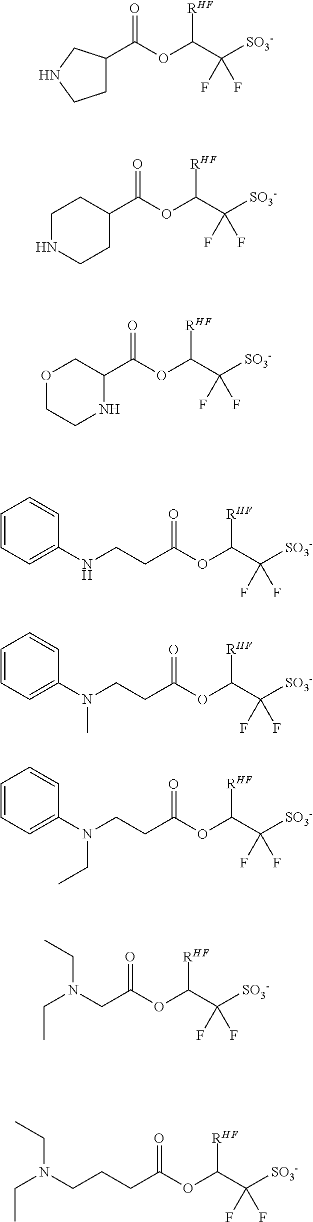

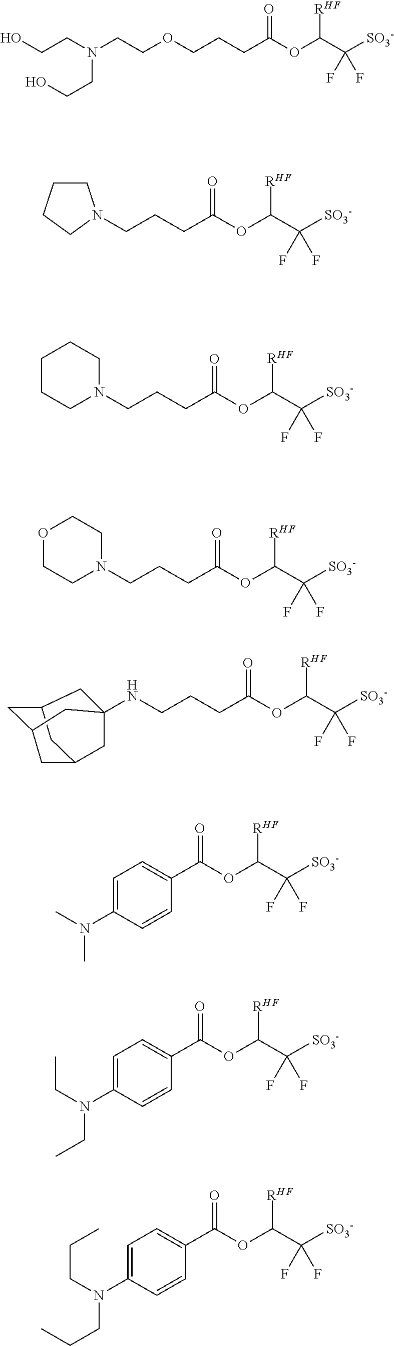

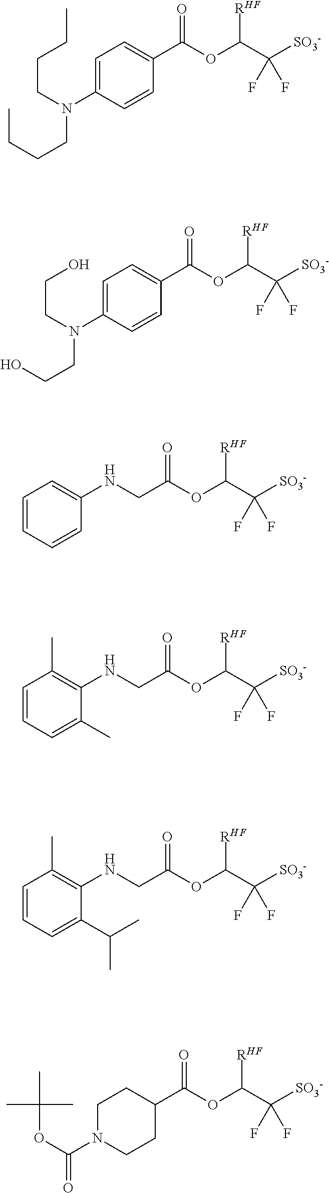

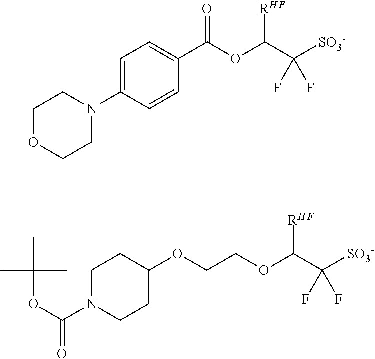

Examples of the cation in the photo-degradable onium salt are as exemplified above for the cation M⁺ in formula (1). Inter alia, the following cations are preferred, but not limitative.

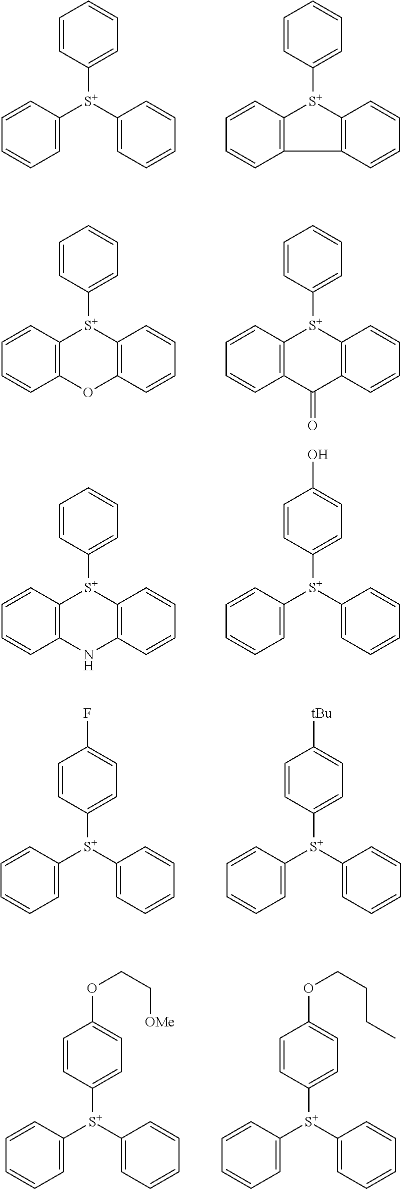

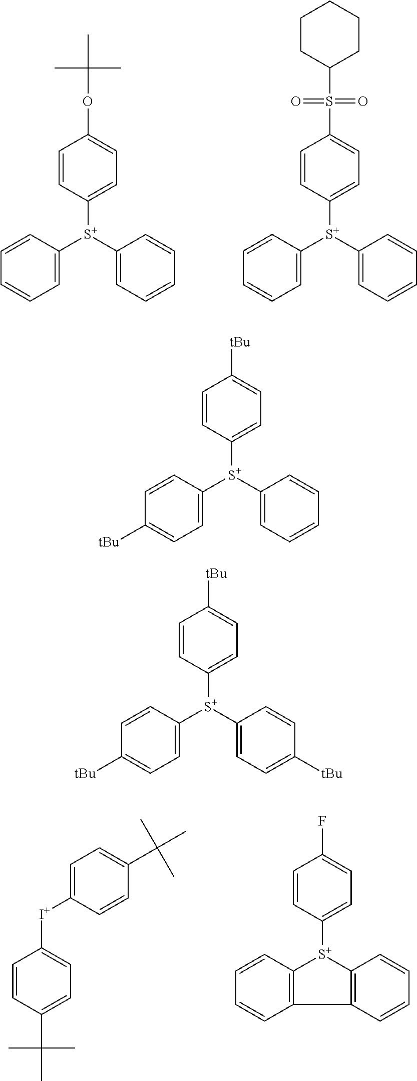 [SMILES: CC(C)(C)Oc1ccc([S+](c2ccccc2)c2ccccc2)cc1 ; O=S(=O)(c1ccc([S+](c2ccccc2)c2ccccc2)cc1)C1CCCCC1 ; CC(C)(C)c1ccc([S+](c2ccccc2)c2ccc(C(C)(C)C)cc2)cc1 ; CC(C)(C)c1ccc([S+](c2ccc(C(C)(C)C)cc2)c2ccc(C(C)(C)C)cc2)cc1 ; CC(C)(C)c1ccc([I+]c2ccc(C(C)(C)C)cc2)cc1 ; Fc1ccc(-[s+]2c3ccccc3c3ccccc32)cc1]

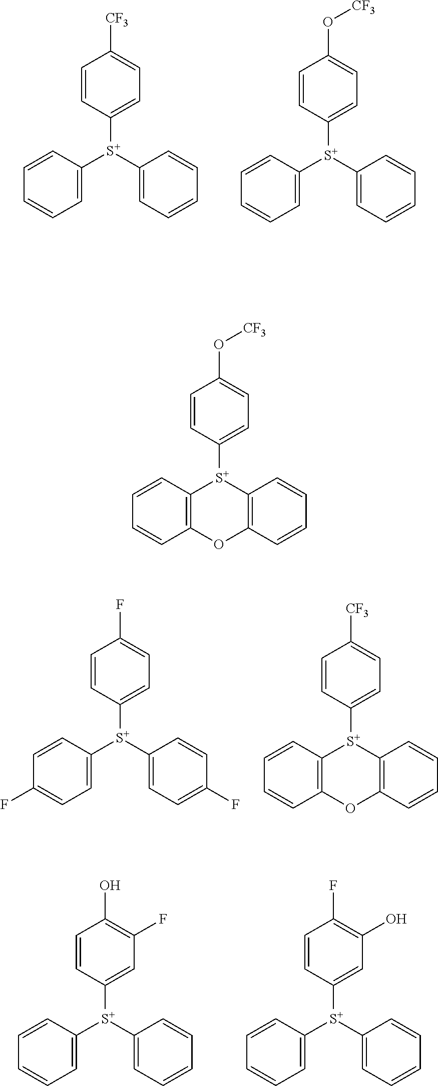

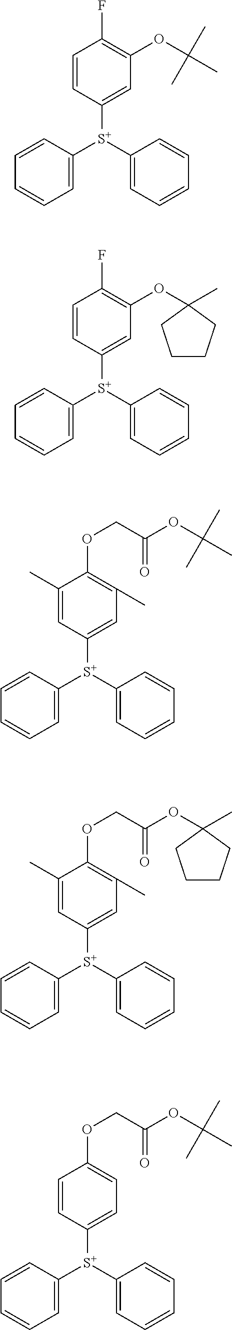 [SMILES: CC(C)(C)Oc1cc([S+](c2ccccc2)c2ccccc2)ccc1F ; CC1(Oc2cc([S+](c3ccccc3)c3ccccc3)ccc2F)CCCC1 ; Cc1cc([S+](c2ccccc2)c2ccccc2)cc(C)c1OCC(=O)OC(C)(C)C ; Cc1cc([S+](c2ccccc2)c2ccccc2)cc(C)c1OCC(=O)OC1(C)CCCC1 ; CC(C)(C)OC(=O)COc1ccc([S+](c2ccccc2)c2ccccc2)cc1]

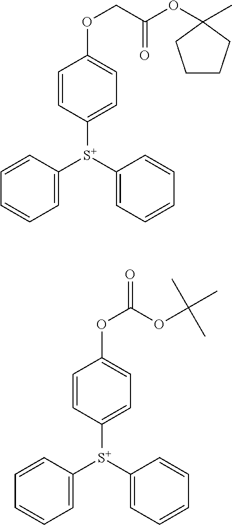 [SMILES: CC1(OC(=O)COc2ccc([S+](c3ccccc3)c3ccccc3)cc2)CCCC1 ; CC(C)(C)OC(=O)Oc1ccc([S+](c2ccccc2)c2ccccc2)cc1]

Examples of the photo-decomposable onium salt include arbitrary combinations of cations with anions, both as exemplified above, but are not limited thereto.

Component (C) is preferably used in an amount of 2 to 30 parts by weight, more preferably 2.5 to 20 parts by weight, even more preferably 4 to 15 parts by weight per 100 parts by weight of the base polymer (A). The acid diffusion inhibitor within the range allows for easy adjustment of resist sensitivity, holds down the diffusion rate of acid within the resist film (with improved resolution), suppresses a sensitivity change after exposure, reduces substrate or environment dependency, and improves exposure latitude and pattern profile. Also the addition of the acid diffusion inhibitor is effective for improving substrate adhesion. It is noted that the amount of component (C) is the total amount of the acid diffusion inhibitor in the form of the onium salt compound having formula (1) and the acid diffusion inhibitor other than the onium salt compound having formula (1). In the acid diffusion inhibitor (C), preferably the onium salt compound having formula (1) accounts for 50 to 100% by weight. The acid diffusion inhibitor as component (C) may be used alone or in admixture.

### (D) Organic Solvent

The resist composition further comprises (D) an organic solvent. The organic solvent used herein is not particularly limited as long as the foregoing and other components are dissolvable therein. Examples of the organic solvent used herein are described in JP-A 2008-111103, paragraphs [0144]-[0145] (U.S. Pat. No. 7,537,880). Exemplary solvents include ketones such as cyclohexanone (CyHO) and methyl-2-n-pentyl ketone; alcohols such as 3-methoxybutanol, 3-methyl-3-methoxybutanol, 1-methoxy-2-propanol, 1-ethoxy-2-propanol, and diacetone alcohol (DAA); ethers such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, t-butyl acetate, t-butyl propionate, and propylene glycol mono-t-butyl ether acetate; and lactones such as y-butyrolactone (GBL), which may be used alone or in admixture. Where an acid labile group of acetal form is used, a high boiling alcohol solvent such as diethylene glycol, propylene glycol, glycerol, 1,4-butanediol or 1,3-butanediol may be added to accelerate deprotection reaction of acetal.

Of these organic solvents, preference is given to 1-ethoxy-2-propanol, PGMEA, DAA, CyHO, and GBL and mixtures thereof because the PAG is highly soluble therein. The preferred solvent system is a mixture of PGMEA as solvent X and at least one of 1-ethoxy-2-propanol, DAA, CyHO, and GBL as solvent Y in a ratio X:Y of from 90:10 to 60:40.

The organic solvent (D) is preferably added in an amount of 100 to 8,000 parts, and more preferably 400 to 6,000 parts by weight per 100 parts by weight of the base polymer (A).

### (E) Surfactant

In addition to the foregoing components, the resist composition may comprise (E) a surfactant which is commonly used for facilitating coating operation.

Component (E) is typically a surfactant which is insoluble or substantially insoluble in water and alkaline developer or a surfactant which is insoluble or substantially insoluble in water and soluble in alkaline developer.

For the surfactant which is insoluble or substantially insoluble in water and alkaline developer, reference should be made to JP-A 2010-215608 and JP-A 2011-016746. Suitable surfactants include FC-4430 (3M), Surflon® S-381, KH-20 and KH-30 (AGC Seimi Chemical Co., Ltd.), and Olfine® E1004 (Nisshin Chemical Co., Ltd.). Partially fluorinated oxetane ring-opened polymers having the structural formula (surf-1) are also useful.

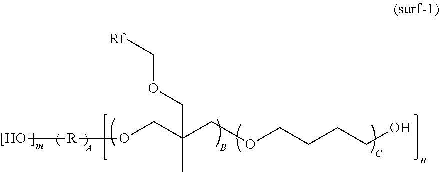

It is provided herein that R, Rf, A, B, C, m, and n are applied to only formula (surf-1), independent of their descriptions other than for the surfactant. R is a di- to tetra-valent C₂-C₅ aliphatic group. Exemplary divalent groups include ethylene, 1,4-butylene, 1,2-propylene, 2,2-dimethyl-1,3-propylene and 1,5-pentylene. Exemplary tri- and tetra-valent groups are shown below.

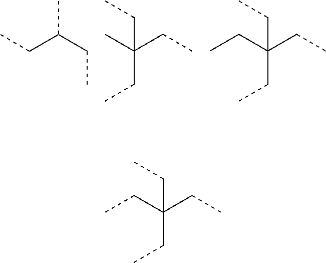

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the above structural formula does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist film surface for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing recurring units of at least one type selected from the formulae (10A) to (10E).

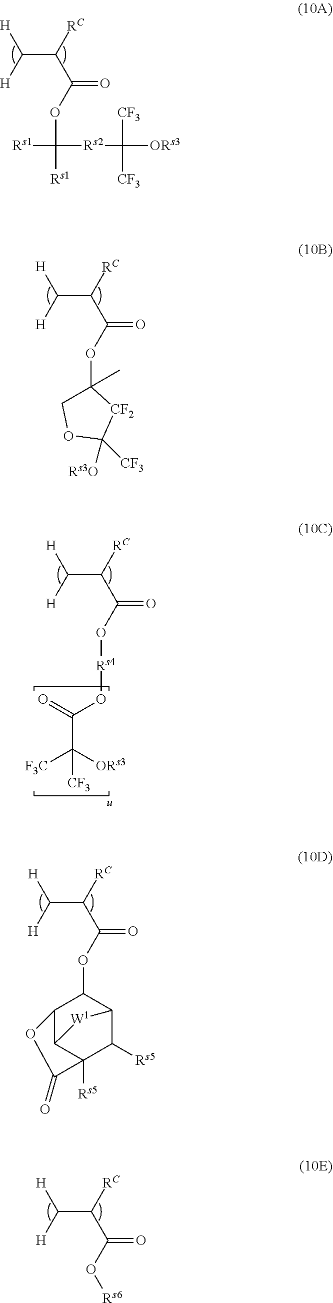

Herein, R^{C} is hydrogen or methyl. W¹ is —CH₂—, —CH₂CH₂— or —O—, or two separate —H. R^{s1} is each independently hydrogen or a C₁-C₁₀ hydrocarbyl group. R^{s2} is a single bond or a C₁-C₅ alkanediyl group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond (—O—) or carbonyl moiety (—C(═O)—) may intervene in a carbon-carbon bond. R^{s4} is a C₁-C₂₀ (u+1)-valent hydrocarbon or fluorinated hydrocarbon group, and u is an integer of 1 to 3. IV is each independently hydrogen or a group having the formula: —C(═O)—O—R^{s5A} wherein R^{s5A} is a C₁-C₂₀ fluorinated hydrocarbyl group. R^{s6} is a C₁-C₁₅ hydrocarbyl or fluorinated hydrocarbyl group in which —O— or —C(═O)— may intervene in a carbon-carbon bond.

The polymeric surfactant may further contain recurring units other than the recurring units having formulae (10A) to (10E). Typical other recurring units are those derived from methacrylic acid and α-trifluoromethylacrylic acid derivatives. In the polymeric surfactant, the content of the recurring units having formulae (10A) to (10E) is preferably at least 20 mol %, more preferably at least 60 mol %, most preferably 100 mol % of the overall recurring units.

For the surfactant which is insoluble or substantially insoluble in water and soluble in alkaline developer, reference may be made to JP-A 2008-122932, JP-A 2009-098638, JP-A 2009-191151, JP-A 2009-192784, JP-A 2009-276363, JP-A 2010-107695, JP-A 2010-134012, JP-A 2010-250105, and JP-A 2011-042789.

The amount of component (E) is preferably 0 to 20 parts by weight per 100 parts by weight of the base polymer (A). When added, the amount of component (E) is more preferably 0.001 to 15 parts by weight, even more preferably 0.01 to 10 parts by weight. The surfactant may be used alone or in admixture. The surfactant is also described in JP-A 2007-297590.

### (F) Other Components

The resist composition may further comprise (F) another component, for example, a compound which is decomposed with an acid to generate another acid (i.e., acid amplifier compound), an organic acid derivative, a fluorinated alcohol, a crosslinker, a compound having a Mw of up to 3,000 which changes its solubility in developer under the action of an acid (i.e., dissolution inhibitor), and an acetylene alcohol. Specifically, the acid amplifier compound is described in JP-A 2009-269953 and JP-A 2010-215608 and preferably used in an amount of 0 to 5 parts, more preferably 0 to 3 parts by weight per 100 parts by weight of the base polymer (A). An extra amount of the acid amplifier compound can make the acid diffusion control difficult and cause degradations to resolution and pattern profile. With respect to the remaining additives, reference should be made to JP-A 2008-122932, paragraphs [0155]-[0182], JP-A 2009-269953 and JP-A 2010-215608.

The chemically amplified resist composition comprising the onium salt compound having formula (1) as an acid diffusion inhibitor, when processed by photolithography using high-energy radiation such as KrF excimer laser, ArF excimer laser, EB or EUV as the energy source, exhibits a high acid diffusion suppressing effect, and forms a pattern at a high contrast and with improved lithography performance factors such as CDU, LWR and sensitivity.

### Process

A further embodiment of the invention is a pattern forming process using the chemically amplified resist composition defined above. The process includes the steps of applying the resist composition to form a resist film on a substrate, exposing a selected region of the resist film to high-energy radiation, and developing the exposed resist film in a developer. Any desired steps may be added to the process if necessary.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The resist composition is applied onto a substrate by a suitable coating technique such as spin coating. The coating is prebaked on a hot plate preferably at a temperature of 60 to 180° C. for 10 to 600 seconds, more preferably at 70 to 150° C. for 15 to 300 seconds. The resulting resist film preferably has a thickness of 10 to 2,000 nm.

The resist film is then exposed to high-energy radiation. On use of KrF excimer laser, ArF excimer laser or EUV of wavelength 13.5 nm, the resist film is exposed through a mask having the desired pattern in a dose of preferably 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid between the mask and the resist film may be employed if desired. In the immersion lithography, preferably a liquid having a refractive index of at least 1.0 is held between the resist film and the projection lens. The liquid is typically water, and in this case, a protective film which is insoluble in water may be formed on the resist film.

While the water-insoluble protective film which is used in the immersion lithography serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate at 60 to 150° C. for 1 to 5 minutes, preferably at 80 to 140° C. for 1 to 3 minutes.

The resist film is then developed with a developer in the form of an aqueous base to solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, a desired resist pattern is formed on the substrate.

With respect to the formation of a positive pattern using an alkaline aqueous solution as the developer, reference may be made to U.S. Pat. No. 8,647,808 (JP-A 2011-231312, paragraphs [0138]-[0146]). With respect to the formation of a negative pattern using an organic solvent as the developer, reference may be made to U.S. Pat. No. 9,256,127 (JP-A 2015-214634, paragraphs [0173]-[0183]).

Any desired step may be added to the pattern forming process. For example, after the resist film is formed, a step of rinsing with pure water (post-soaking) may be introduced to extract the acid generator or the like from the film surface or wash away particles. After exposure, a step of rinsing (post-soaking) may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

Where a hole pattern is formed by negative tone development using organic solvent developer, exposure by double dipole illuminations of X- and Y-direction line patterns provides the highest contrast light. The contrast may be further increased by combining two dipole illuminations of X- and Y-direction line patterns with s-polarized illumination. These pattern forming processes are described in JP-A 2011-221513.

With respect to the developer in the pattern forming process, examples of the aqueous alkaline solution include TMAH aqueous solutions as mentioned above and aqueous alkaline solutions described in JP-A 2015-180748, paragraphs [0148]-[0149], preferably 2 to 3% by weight TMAH aqueous solutions.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. These organic solvents may be used alone or in admixture of two or more.

A hole or trench pattern after development may be shrunk by the thermal flow, RELACS® (resolution enhancement lithography assisted by chemical shrink) or DSA (directed self-assembly) process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

When processed by photolithography, the chemically amplified resist composition comprising the onium salt compound having formula (1) as an acid diffusion inhibitor forms a fine size pattern with improved lithography performance factors such as CDU, LWR and sensitivity.

### EXAMPLES

Examples of the invention are given below by way of illustration and not by way of limitation. The abbreviation “pbw” is parts by weight. For all polymers, Mw and Mn are determined by GPC versus polystyrene standards using tetrahydrofuran (THF) solvent.

### Example 1-1

Synthesis of Acid Diffusion Inhibitor Q-1

### (1) Synthesis of Compound SM-1

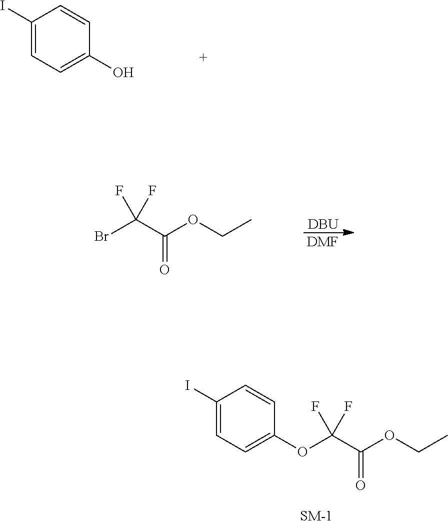

A reactor was charged with 4.4 g of 4-iodophenol, 4.1 g of ethyl bromodifluoroacetate, 3.1 g of diazabicycloundecene, and 30 g of N,N-dimethylformamide, which were stirred at 70° C. overnight. After the disappearance of reactants was confirmed by ¹⁹F-NMR spectroscopy, under ice cooling, 60 g of 5 wt % hydrochloric acid was added to the reaction solution to quench the reaction. 40 g of toluene was added to the reaction solution. At the end of stirring, the organic layer was taken out and washed with 40 g of deionized water and 40 g of 25 wt % methanol aqueous solution. The organic layer was concentrated under reduced pressure, obtaining the desired Compound SM-1 as crude oily matter (amount 5.8 g). The compound was used in the subsequent step without purification.

### (2) Synthesis of Compound SM-2

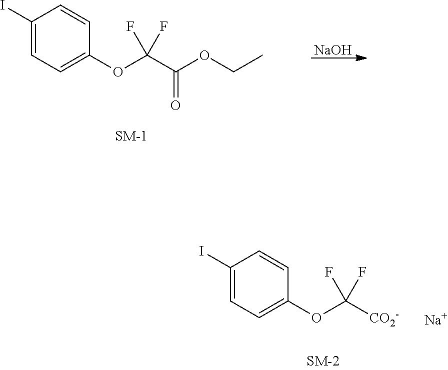

A reactor was charged with 5.8 g of Compound SM-1, 2.2 g of 25 wt % sodium hydroxide aqueous solution, and 20 g of 1,4-dioxane, which were stirred at room temperature overnight. The reaction solution was concentrated under reduced pressure, 35 g of tert-butyl methyl ether was added to the concentrate, and the mixture was stirred for 20 minutes. The solid precipitate was collected by filtration, washed with tert-butyl methyl ether, and dried, obtaining the desired Compound SM-2 (amount 5.2 g, yield 58%).

### (3) Synthesis of Acid Diffusion Inhibitor Q-1

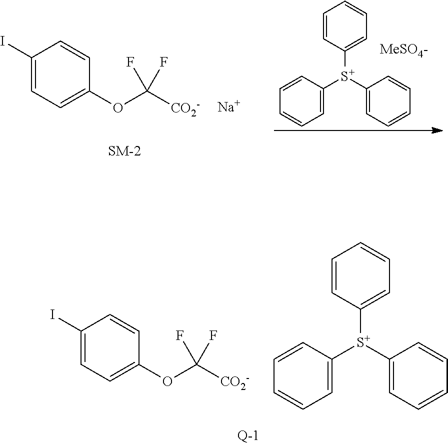

A reactor was charged with 5.2 g of Compound SM-2, 5.2 g of triphenylsulfonium methylsulfate, 40 g of methylene chloride, and 20 g of deionized water, which were stirred at room temperature for 2 hours. The organic layer was taken out, washed with 20 g of deionized water, and concentrated under reduced pressure. 40 g of diisopropyl ether was added to the concentrate and stirred for 30 minutes for crystallization. The solid precipitate was filtered, washed with diisopropyl ether, and dried at 50° C. in vacuum, obtaining the target acid diffusion inhibitor Q-1 as white solid (amount 6.1 g, yield 91%). The spectral data of Q-1 are shown below.

¹H-NMR (500 MHz, DMSO-d₆):

δ=6.91 (2H, m), 7.63 (2H, m), 7.75-7.87 (15H, m) ppm

¹⁹F-NMR (500 MHz, DMSO-d6):

δ=−76.5 (2F, s) ppm

IR (D-ATR):

ν=3084, 3042, 1669, 1577, 1476, 1447, 1389, 1343, 1327, 1300, 1207, 1161, 1130, 1037, 1001, 932, 870, 846, 835, 804, 764, 751, 745, 702, 685, 585, 552, 507 cm⁻¹

Time-of-flight mass spectrometry (TOFMS; MALDI)

Positive M⁺ 263.1 (corresponding to C₁₈H₁₅S⁺)

Negative M⁻ 312.9 (corresponding to C₈H₄F₂IO₃⁻)

### Example 1-2

Synthesis of Acid Diffusion Inhibitor Q-2

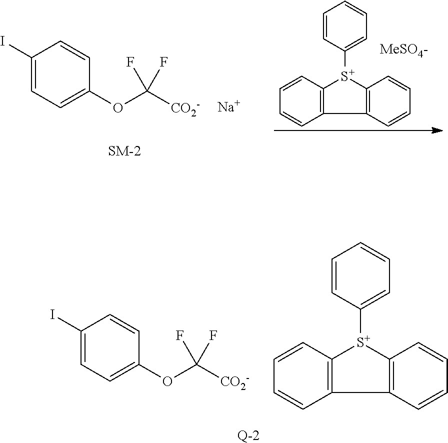

A reactor was charged with 4.4 g of Compound SM-2, 5.9 g of S-phenyldibenzothiophenium methylsulfate, 40 g of methylene chloride, and 20 g of deionized water, which were stirred at room temperature for 2 hours. The organic layer was taken out, washed with 40 g of deionized water, 40 g of 0.3 wt % ammonia water, and 40 g of deionized water. The organic layer was concentrated under reduced pressure, allowing solids to precipitate. The solid precipitate was dispersed in 20 g of diisopropyl ether, followed by 20 minutes of stirring. The solid was filtered, washed with diisopropyl ether, and dried at 50° C. in vacuum, obtaining the target acid diffusion inhibitor Q-1 as white solid (amount 6.8 g, yield 91%). The spectral data of Q-2 are shown below.

¹H-NMR (500 MHz, DMSO-d6):

δ=6.91 (2H, m), 7.55-7.64 (6H, m), 7.68 (1H, m), 7.74 (2H, m), 7.94 (2H, m), 8.38 (2H, d), 8.52 (2H, dd) ppm

¹⁹F-NMR (500 MHz, DMSO-d6):

δ=−76.1 (2F, s) ppm

IR (D-ATR):

ν=3499, 3411, 3273, 3100, 3061, 1653, 1575, 1482, 1448, 1428, 1403, 1389, 1293, 1275, 1218, 1181, 1166, 1138, 1106, 1090, 1057, 1009, 997, 873, 846, 826, 800, 778, 758, 751, 734, 707, 699, 680, 612, 524, 501, 488 cm⁻¹

TOFMS; MALDI

Positive M⁺ 261.1 (corresponding to C₁₈H₁₃S⁺)

Negative M⁻ 312.9 (corresponding to C₈H₄F₂IO₃⁻)

### Examples 1-3 to 1-28

Synthesis of Acid Diffusion Inhibitors Q-3 to Q-28

Acid diffusion inhibitors Q-3 to Q-28 as shown below were synthesized in accordance with Examples 1-1 and 1-2.

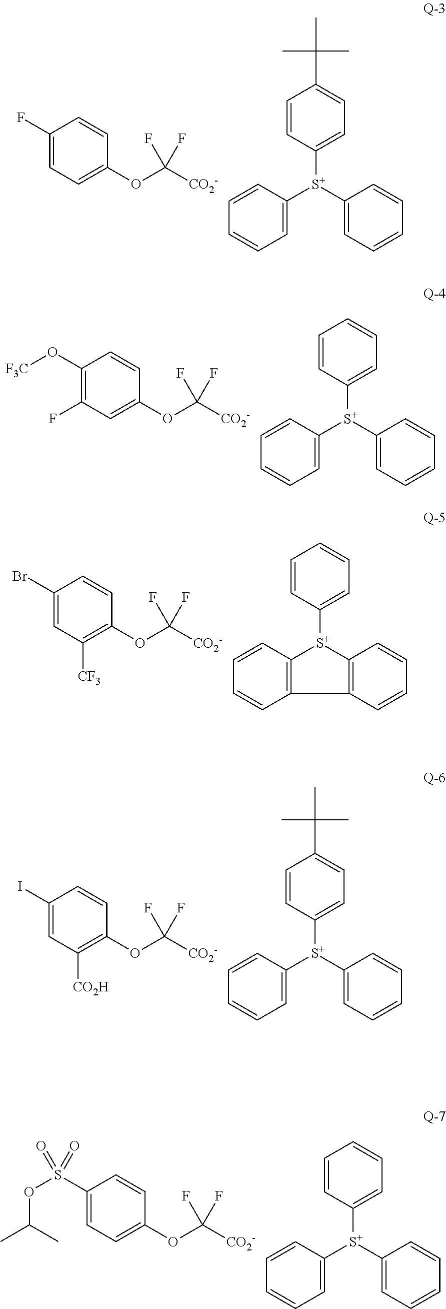

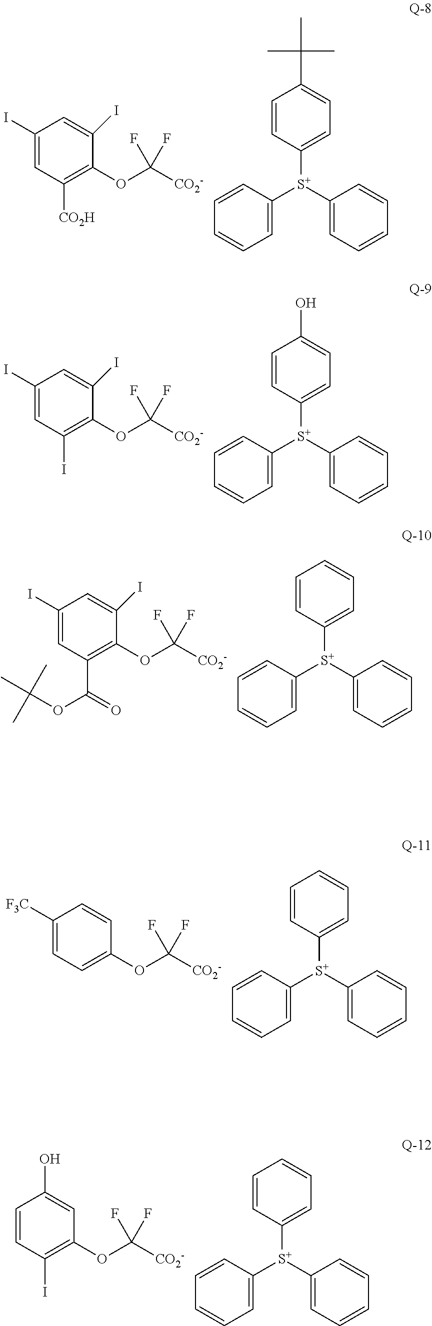

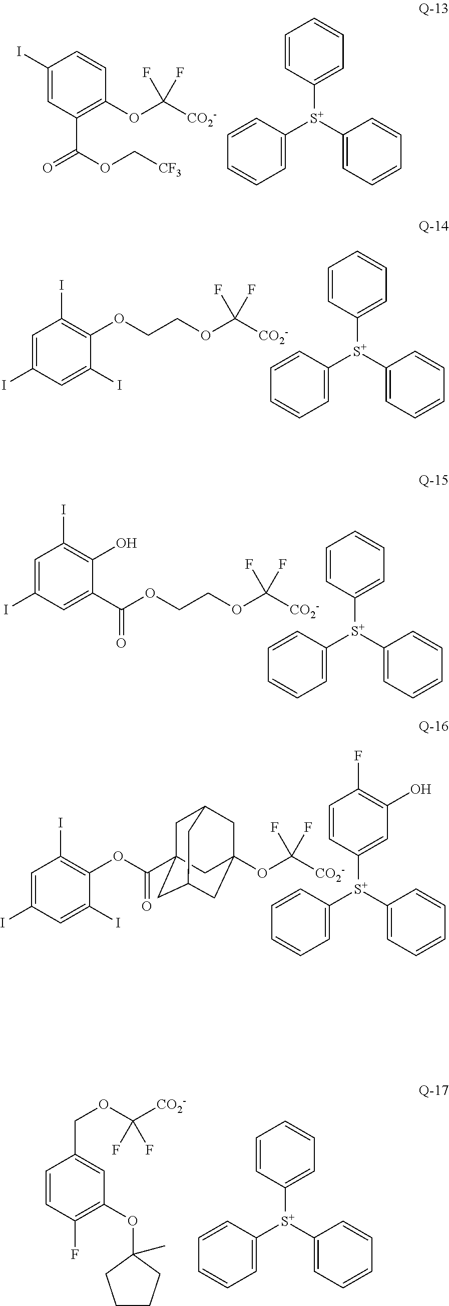

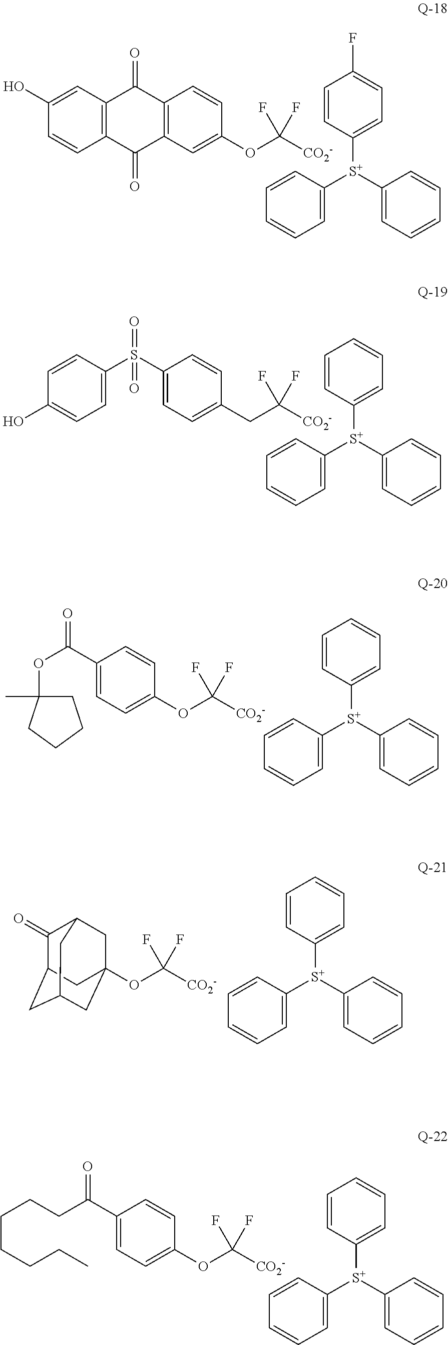

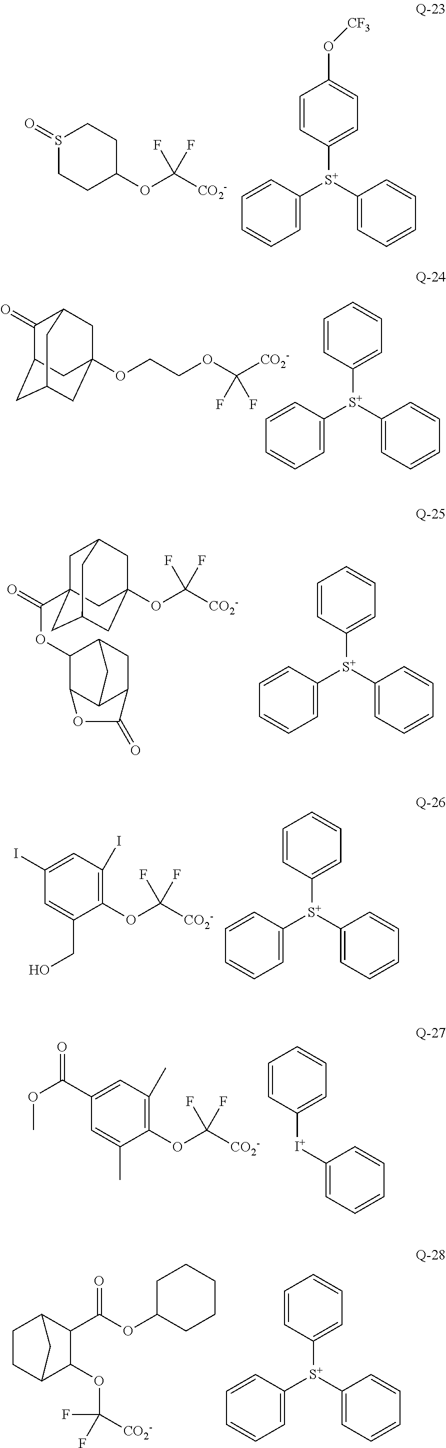

### Synthesis Example 1

Synthesis of Polymer P-1

In nitrogen atmosphere, 22 g of 1-tert-butylcyclopentyl methacrylate, 17 g of 2-oxotetrahydrofuran-3-yl methacrylate, 0.48 g of dimethyl 2,2′-azobis(2-methylpropionate) (V-601 by Wako Pure Chemical Industries, Ltd.), 0.41 g of 2-mercaptoethanol, and 50 g of methyl ethyl ketone were combined to form a monomer/initiator solution. A flask in nitrogen atmosphere was charged with 23 g of methyl ethyl ketone, which was heated at 80° C. with stirring. With stirring, the monomer/initiator solution was added dropwise to the flask over 4 hours. After the completion of dropwise addition, the polymerization solution was continuously stirred for 2 hours while maintaining the temperature of 80° C. The polymerization solution was cooled to room temperature, whereupon it was added dropwise to 640 g of methanol with vigorous stirring. The solid precipitate was collected by filtration, washed twice with 240 g of methanol, and vacuum dried at 50° C. for 20 hours, obtaining Polymer P-1 in white powder form (amount 36 g, yield 90%). Polymer P-1 had a Mw of 8,500 and a dispersity Mw/Mn of 1.63.

| | | |
|---|---|---|
| Polymer P- 1 (a = 50, b = 50) MW = 8,500 Mw/Mn = 1.63 | 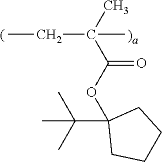 | 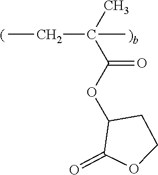 |

### Synthesis Examples 2 to 4

Synthesis of Polymers P-2 to P-4

Polymers P-2 to P-4 were synthesized by the same procedure as in Synthesis Example 1 aside from changing the type and amount of monomers.

| | | | |
|---|---|---|---|
| Polymer P-2 (a = 60, b = 40) Mw = 8,900 Mw/Mn = 1.82 | 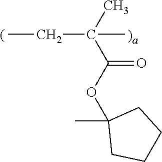 | 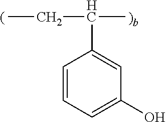 | |
| Polymer P-3 (a = 40, b = 10, c = 5, d = 35, e = 10) Mw = 9,600 Mw/Mn = 1.75 | 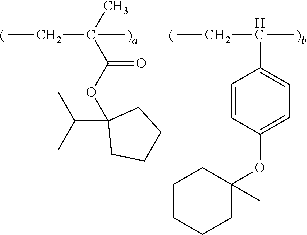 | 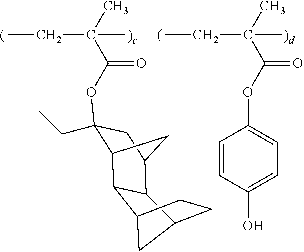 | 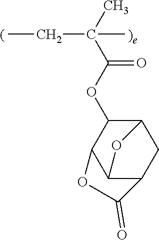 |

| | | | |
|---|---|---|---|
| Polymer P-4 (a = 55, b = 30, c = 15) Mw = 12,100 Mw/Mn = 1.63 | 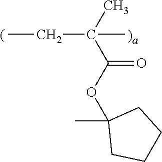 | 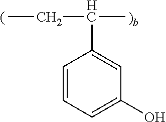 | 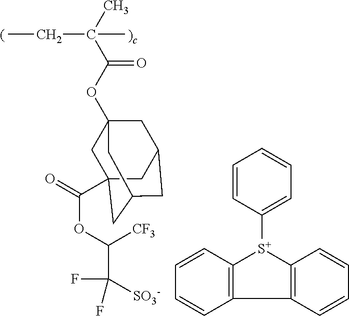 |

### Examples 2-1 to 2-79 and Comparative Examples 1-1 to 1-37

Preparation of Chemically Amplified Resist Compositions

Chemically amplified resist compositions were prepared by dissolving the components shown in Tables 1 to 5 in a solvent containing 0.01 wt % of surfactant Polyfox 636 (Omnova Solutions, Inc.), and filtering the solution through a Teflon® filter with a pore size of 0.2 μm.

The photoacid generators PAG-1 to PAG-3, solvents, comparative acid diffusion inhibitors Q-A to Q-O, and alkali-soluble surfactant SF-1 in Tables 1 to 5 are identified below.

### Photoacid Generators PAG-1 to PAG-3:

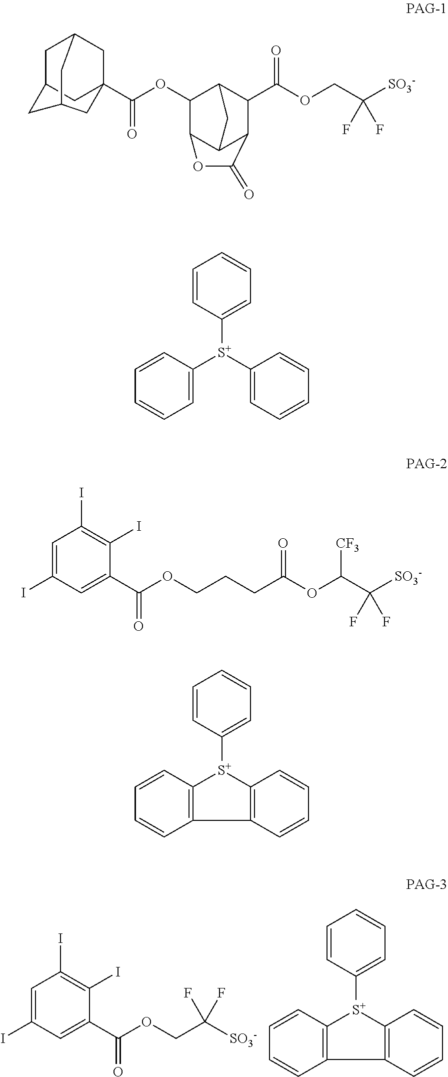

### Solvent:

PGMEA=propylene glycol monomethyl ether acetate

GBL=γ-butyrolactone

CyHO=cyclohexanone

DAA=diacetone alcohol

### Acid Diffusion Inhibitors Q-A to Q-O:

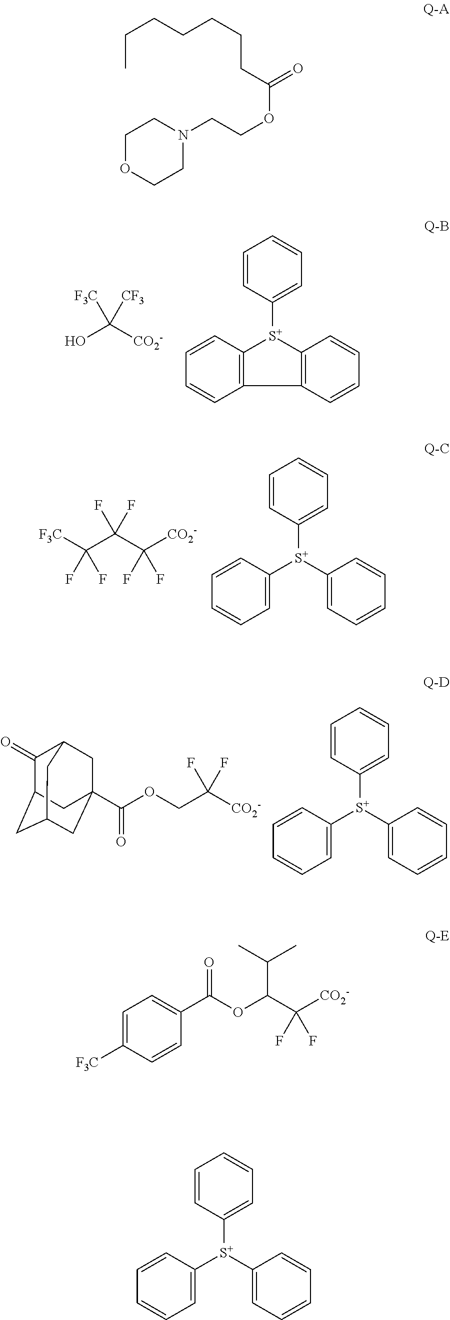

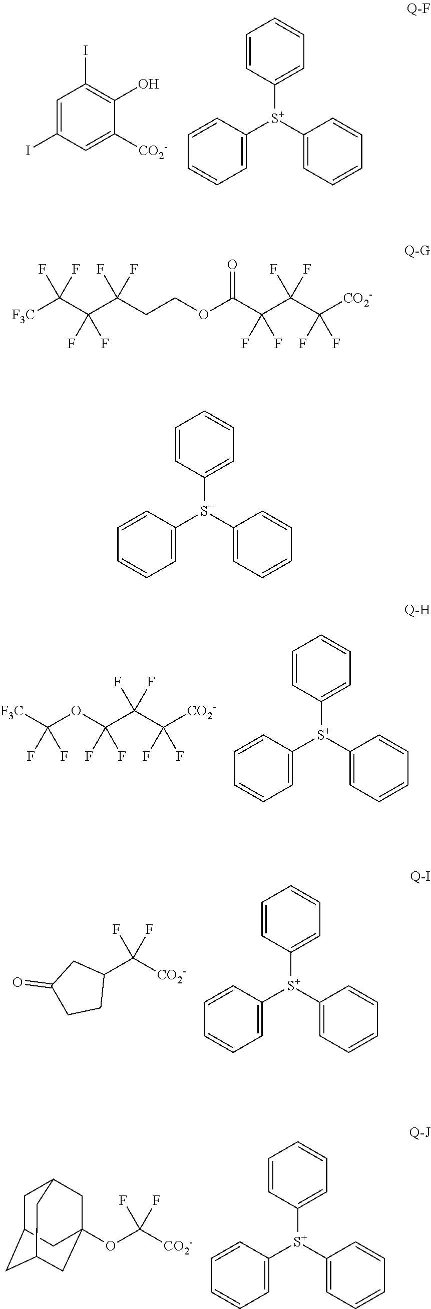

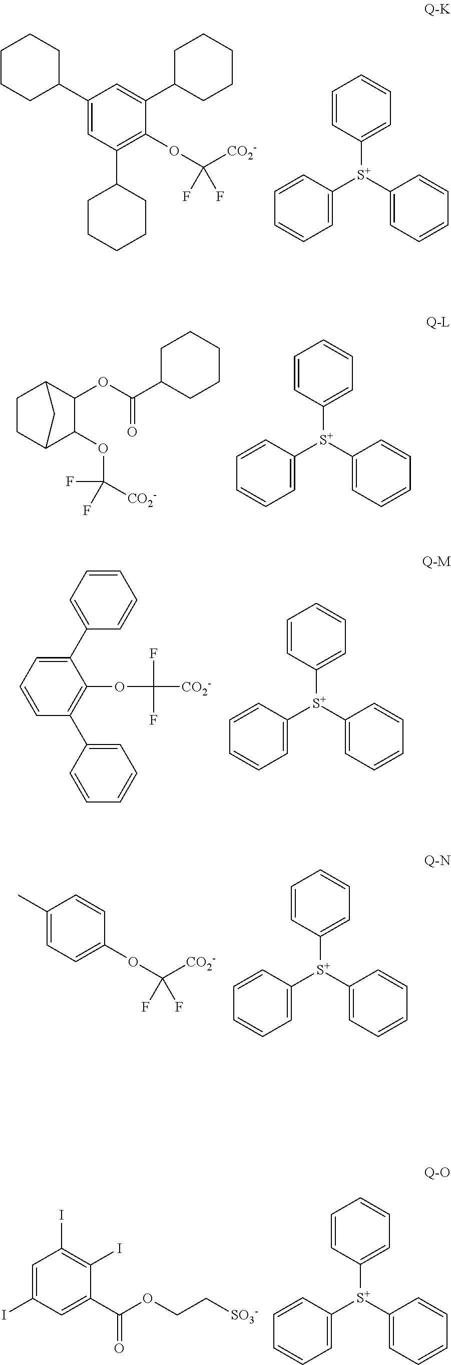

### Alkali-Soluble Surfactant SF-1:

### poly(2,2,3,3,4,4,4-heptafluoro-1-isobutyl-1-butyl methacrylate/9-(2,2,2-trifluoro-1-trifluoromethylethyloxycarbonyl)-4-oxatricyclo[4.2.1.0^{3,7}]nonan-5-on-2-yl methacrylate)

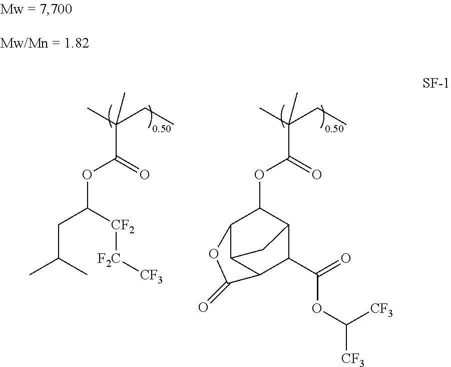

TABLE 1

| | | | | Photoacid | Acid diffusion | | |
|---|---|---|---|---|---|---|---|
| | | Resist | Polymer | generator | inhibitor | Surfactant | Solvent |
| | | composition | (pbw) | (pbw) | (pbw) | (pbw) | (pbw) |
| Example | 2-1 | R-1 | P-1 | PAG-1 | Q-1 | SF-1 | PGMEA/GBL |
| | | | (100) | (8.0) | (5.0) | (3.0) | (1,920/480) |
| | 2-2 | R-2 | P-1 | PAG-1 | Q-3 | SF-1 | PGMEA/GBL |
| | | | (100) | (8.0) | (5.0) | (3.0) | (1,920/480) |
| | 2-3 | R-3 | P-1 | PAG-1 | Q-6 | SF-1 | PGMEA/GBL |
| | | | (100) | (8.0) | (4.9) | (3.0) | (1,920/480) |
| | 2-4 | R-4 | P-1 | PAG-1 | Q-9 | SF-1 | PGMEA/GBL |
| | | | (100) | (8.0) | (4.7) | (3.0) | (1,920/480) |
| | 2-5 | R-5 | P-1 | PAG-1 | Q-11 | SF-1 | PGMEA/GBL |
| | | | (100) | (8.0) | (5.0) | (3.0) | (1,920/480) |
| | 2-6 | R-6 | P-1 | PAG-1 | Q-17 | SF-1 | PGMEA/GBL |
| | | | (100) | (8.0) | (5.0) | (3.0) | (1,920/480) |
| | 2-7 | R-7 | P-1 | PAG-1 | Q-21 | SF-1 | PGMEA/GBL |
| | | | (100) | (8.0) | (5.0) | (3.0) | (1,920/480) |
| | 2-8 | R-8 | P-1 | PAG-1 | Q-22 | SF-1 | PGMEA/GBL |
| | | | (100) | (8.0) | (4.7) | (3.0) | (1,920/480) |
| | 2-9 | R-9 | P-1 | PAG-1 | Q-23 | SF-1 | PGMEA/GBL |
| | | | (100) | (8.0) | (4.8) | (3.0) | (1,920/480) |
| | 2-10 | R-10 | P-1 | PAG-1 | Q-24 | SF-1 | PGMEA/GBL |
| | | | (100) | (8.0) | (5.0) | (3.0) | (1,920/480) |
| | 2-11 | R-11 | P-1 | PAG-1 | Q-25 | SF-1 | PGMEA/GBL |
| | | | (100) | (8.0) | (5.0) | (3.0) | (1,920/480) |
| | 2-12 | R-12 | P-1 | PAG-1 | Q-26 | SF-1 | PGMEA/GBL |
| | | | (100) | (8.0) | (4.9) | (3.0) | (1,920/480) |
| | 2-13 | R-13 | P-1 | PAG-1 | Q-28 | SF-1 | PGMEA/GBL |
| | | | (100) | (8.0) | (5.0) | (3.0) | (1,920/480) |
| | 2-14 | R-14 | P-1 | PAG-1 | Q-1 (3.3) | SF-1 | PGMEA/GBL |
| | | | (100) | (8.0) | Q-A (0.8) | (3.0) | (1,920/480) |
| | 2-15 | R-15 | P-1 | PAG-1 | Q-21 (3.8) | SF-1 | PGMEA/GBL |
| | | | (100) | (8.0) | Q-B (1.1) | (3.0) | (1,920/480) |
| | 2-16 | R-16 | P-1 | PAG-1 | Q-28 (3.8) | SF-1 | PGMEA/GBL |
| | | | (100) | (8.0) | Q-B (1.1) | (3.0) | (1,920/480) |
| | 2-17 | R-17 | P-2 | PAG-2 | Q-1 | SF-1 | PGMEA/DAA |
| | | | (100) | (20.0) | (10.0) | (3.0) | (2,100/900) |
| | 2-18 | R-18 | P-2 | PAG-2 | Q-2 | SF-1 | PGMEA/DAA |
| | | | (100) | (20.0) | (10.0) | (3.0) | (2,100/900) |
| | 2-19 | R-19 | P-2 | PAG-2 | Q-3 | SF-1 | PGMEA/DAA |
| | | | (100) | (20.0) | (9.5) | (3.0) | (2,100/900) |
| | 2-20 | R-20 | P-2 | PAG-2 | Q-4 | SF-1 | PGMEA/DAA |
| | | | (100) | (20.0) | (10.0) | (3.0) | (2,100/900) |
| | 2-21 | R-21 | P-2 | PAG-2 | Q-5 | SF-1 | PGMEA/DAA |
| | | | (100) | (20.0) | (10.0) | (3.0) | (2,100/900) |
| | 2-22 | R-22 | P-2 | PAG-2 | Q-6 | SF-1 | PGMEA/DAA |
| | | | (100) | (20.0) | (9.8) | (3.0) | (2,100/900) |
| | 2-23 | R-23 | P-2 | PAG-2 | Q-7 | SF-1 | PGMEA/DAA |
| | | | (100) | (20.0) | (10.0) | (3.0) | (2,100/900) |
| | 2-24 | R-24 | P-2 | PAG-2 | Q-8 | SF-1 | PGMEA/DAA |
| | | | (100) | (20.0) | (9.8) | (3.0) | (2,100/900) |
| | 2-25 | R-25 | P-2 | PAG-2 | Q-9 | SF-1 | PGMEA/DAA |
| | | | (100) | (20.0) | (9.9) | (3.0) | (2,100/900) |
| | 2-26 | R-26 | P-2 | PAG-2 | Q-10 | SF-1 | PGMEA/DAA |
| | | | (100) | (20.0) | (10.0) | (3.0) | (2,100/900) |
| | 2-27 | R-27 | P-2 | PAG-2 | Q-11 | SF-1 | PGMEA/DAA |
| | | | (100) | (20.0) | (10.0) | (3.0) | (2,100/900) |

TABLE 2

| | | | | Photoacid | Acid diffusion | | |
|---|---|---|---|---|---|---|---|
| | | Resist | Polymer | generator | inhibitor | Surfactant | Solvent |
| | | composition | (pbw) | (pbw) | (pbw) | (pbw) | (pbw) |
| Example | 2-28 | R-28 | P-2 | PAG-2 | Q-12 | SF-1 | PGMEA/DAA |
| | | | (100) | (20.0) | (10.0) | (3.0) | (2,100/900) |
| | 2-29 | R-29 | P-2 | PAG-2 | Q-13 | SF-1 | PGMEA/DAA |
| | | | (100) | (20.0) | (10.0) | (3.0) | (2,100/900) |
| | 2-30 | R-30 | P-2 | PAG-2 | Q-14 | SF-1 | PGMEA/DAA |
| | | | (100) | (20.0) | (9.7) | (3.0) | (2,100/900) |
| | 2-31 | R-31 | P-2 | PAG-2 | Q-15 | SF-1 | PGMEA/DAA |
| | | | (100) | (20.0) | (9.9) | (3.0) | (2,100/900) |
| | 2-32 | R-32 | P-2 | PAG-2 | Q-16 | SF-1 | PGMEA/DAA |
| | | | (100) | (20.0) | (9.4) | (3.0) | (2,100/900) |
| | 2-33 | R-33 | P-2 | PAG-2 | Q-17 | SF-1 | PGMEA/DAA |
| | | | (100) | (20.0) | (10.0) | (3.0) | (2,100/900) |
| | 2-34 | R-34 | P-2 | PAG-2 | Q-18 | SF-1 | PGMEA/DAA |
| | | | (100) | (20.0) | (9.5) | (3.0) | (2,100/900) |
| | 2-35 | R-35 | P-2 | PAG-2 | Q-19 | SF-1 | PGMEA/DAA |
| | | | (100) | (20.0) | (10.0) | (3.0) | (2,100/900) |
| | 2-36 | R-36 | P-2 | PAG-2 | Q-20 | SF-1 | PGMEA/DAA |
| | | | (100) | (20.0) | (10.0) | (3.0) | (2,100/900) |
| | 2-37 | R-37 | P-2 | PAG-2 | Q-21 | SF-1 | PGMEA/DAA |
| | | | (100) | (20.0) | (10.0) | (3.0) | (2,100/900) |
| | 2-38 | R-38 | P-2 | PAG-2 | Q-22 | SF-1 | PGMEA/DAA |
| | | | (100) | (20.0) | (9.8) | (3.0) | (2,100/900) |
| | 2-39 | R-39 | P-2 | PAG-2 | Q-23 | SF-1 | PGMEA/DAA |
| | | | (100) | (20.0) | (9.4) | (3.0) | (2,100/900) |
| | 2-40 | R-40 | P-2 | PAG-2 | Q-24 | SF-1 | PGMEA/DAA |
| | | | (100) | (20.0) | (10.0) | (3.0) | (2,100/900) |
| | 2-41 | R-41 | P-2 | PAG-2 | Q-25 | SF-1 | PGMEA/DAA |
| | | | (100) | (20.0) | (9.9) | (3.0) | (2,100/900) |
| | 2-42 | R-42 | P-2 | PAG-2 | Q-26 | SF-1 | PGMEA/DAA |
| | | | (100) | (20.0) | (10.0) | (3.0) | (2,100/900) |
| | 2-43 | R-43 | P-2 | PAG-2 | Q-27 | SF-1 | PGMEA/DAA |
| | | | (100) | (20.0) | (10.0) | (3.0) | (2,100/900) |
| | 2-44 | R-44 | P-2 | PAG-2 | Q-28 | SF-1 | PGMEA/DAA |
| | | | (100) | (20.0) | (10.0) | (3.0) | (2,100/900) |
| | 2-45 | R-45 | P-2 | PAG-2 | Q-1 (6.5) | SF-1 | PGMEA/DAA |
| | | | (100) | (20.0) | Q-B (3.5) | (3.0) | (2,100/900) |
| | 2-46 | R-46 | P-2 | PAG-2 | Q-9 (5.5) | SF-1 | PGMEA/DAA |
| | | | (100) | (20.0) | Q-B (4.5) | (3.0) | (2,100/900) |
| | 2-47 | R-47 | P-2 | PAG-2 | Q-22 (3.5) | SF-1 | PGMEA/DAA |
| | | | (100) | (20.0) | Q-B (6.4) | (3.0) | (2,100/900) |
| | 2-48 | R-48 | P-2 | PAG-3 | Q-2 | SF-1 | PGMEA/DAA/CyHO |
| | | | (100) | (20.0) | (9.5) | (3.0) | (2,100/600/300) |
| | 2-49 | R-49 | P-2 | PAG-3 | Q-8 | SF-1 | PGMEA/DAA/CyHO |
| | | | (100) | (20.0) | (9.3) | (3.0) | (2,100/600/300) |
| | 2-50 | R-50 | P-2 | PAG-3 | Q-14 (6.7) | SF-1 | PGMEA/DAA/CyHO |
| | | | (100) | (20.0) | Q-B (3.0) | (3.0) | (2,100/600/300) |
| | 2-51 | R-51 | P-3 | PAG-2 | Q-1 | SF-1 | PGMEA/DAA |
| | | | (100) | (20.0) | (9.0) | (3.0) | (2,100/900) |
| | 2-52 | R-52 | P-3 | PAG-3 | Q-2 | SF-1 | PGMEA/DAA/CyHO |
| | | | (100) | (20.0) | (8.7) | (3.0) | (2,100/600/300) |
| | 2-53 | R-53 | P-3 | PAG-3 | Q-10 | SF-1 | PGMEA/DAA/CyHO |
| | | | (100) | (20.0) | (8.5) | (3.0) | (2,100/600/300) |
| | 2-54 | R-54 | P-3 | PAG-3 | Q-19 | SF-1 | PGMEA/DAA/CyHO |
| | | | (100) | (20.0) | (8.2) | (3.0) | (2,100/600/300) |

TABLE 3

| | | | | Photoacid | Acid diffusion | | |
|---|---|---|---|---|---|---|---|
| | | Resist | Polymer | generator | inhibitor | Surfactant | Solvent |
| | | composition | (pbw) | (pbw) | (pbw) | (pbw) | (pbw) |
| Example | 2-55 | R-55 | P-4 | — | Q-1 | SF-1 | PGMEA/DAA |
| | | | (100) | | (10.0) | (3.0) | (2,100/900) |
| | 2-56 | R-56 | P-4 | — | Q-2 | SF-1 | PGMEA/DAA |
| | | | (100) | | (10.0) | (3.0) | (2,100/900) |
| | 2-57 | R-57 | P-4 | — | Q-3 | SF-1 | PGMEA/DAA |
| | | | (100) | | (9.9) | (3.0) | (2,100/900) |
| | 2-58 | R-58 | P-4 | — | Q-6 | SF-1 | PGMEA/DAA |
| | | | (100) | | (9.8) | (3.0) | (2,100/900) |
| | 2-59 | R-59 | P-4 | — | Q-7 | SF-1 | PGMEA/DAA |
| | | | (100) | | (9.9) | (3.0) | (2,100/900) |
| | 2-60 | R-60 | P-4 | — | Q-8 | SF-1 | PGMEA/DAA |
| | | | (100) | | (9.9) | (3.0) | (2,100/900) |
| | 2-61 | R-61 | P-4 | — | Q-9 | SF-1 | PGMEA/DAA |
| | | | (100) | | (9.8) | (3.0) | (2,100/900) |
| | 2-62 | R-62 | P-4 | — | Q-10 | SF-1 | PGMEA/DAA |
| | | | (100) | | (10.0) | (3.0) | (2,100/900) |
| | 2-63 | R-63 | P-4 | — | Q-11 | SF-1 | PGMEA/DAA |
| | | | (100) | | (10.0) | (3.0) | (2,100/900) |
| | 2-64 | R-64 | P-4 | — | Q-12 | SF-1 | PGMEA/DAA |
| | | | (100) | | (10.0) | (3.0) | (2,100/900) |
| | 2-65 | R-65 | P-4 | — | Q-13 | SF-1 | PGMEA/DAA |
| | | | (100) | | (10.0) | (3.0) | (2,100/900) |
| | 2-66 | R-66 | P-4 | — | Q-14 | SF-1 | PGMEA/DAA |
| | | | (100) | | (9.7) | (3.0) | (2,100/900) |
| | 2-67 | R-67 | P-4 | — | Q-15 | SF-1 | PGMEA/DAA |
| | | | (100) | | (10.0) | (3.0) | (2,100/900) |
| | 2-68 | R-68 | P-4 | — | Q-17 | SF-1 | PGMEA/DAA |
| | | | (100) | | (10.0) | (3.0) | (2,100/900) |
| | 2-69 | R-69 | P-4 | — | Q-19 | SF-1 | PGMEA/DAA |
| | | | (100) | | (9.5) | (3.0) | (2,100/900) |
| | 2-70 | R-70 | P-4 | — | Q-20 | SF-1 | PGMEA/DAA |
| | | | (100) | | (10.0) | (3.0) | (2,100/900) |
| | 2-71 | R-71 | P-4 | — | Q-21 | SF-1 | PGMEA/DAA |
| | | | (100) | | (10.0) | (3.0) | (2,100/900) |
| | 2-72 | R-72 | P-4 | — | Q-22 | SF-1 | PGMEA/DAA |
| | | | (100) | | (9.8) | (3.0) | (2,100/900) |
| | 2-73 | R-73 | P-4 | — | Q-26 | SF-1 | PGMEA/DAA |
| | | | (100) | | (10.0) | (3.0) | (2,100/900) |
| | 2-74 | R-74 | P-4 | — | Q-28 | SF-1 | PGMEA/DAA |
| | | | (100) | | (10.0) | (3.0) | (2,100/900) |
| | 2-75 | R-75 | P-4 | PAG-2 | Q-1 | SF-1 | PGMEA/DAA |
| | | | (100) | (5.0) | (17.0) | (3.0) | (2,100/900) |
| | 2-76 | R-76 | P-4 | PAG-2 | Q-2 | SF-1 | PGMEA/DAA |
| | | | (100) | (5.0) | (17.0) | (3.0) | (2,100/900) |
| | 2-77 | R-77 | P-4 | PAG-2 | Q-1 (11.2) | SF-1 | PGMEA/DAA |
| | | | (100) | (5.0) | Q-B (4.8) | (3.0) | (2,100/900) |
| | 2-78 | R-78 | P-4 | PAG-3 | Q-1 | SF-1 | PGMEA/DAA/CyHO |
| | | | (100) | (5.0) | (17.0) | (3.0) | (2,100/600/300) |
| | 2-79 | R-79 | P-4 | PAG-3 | Q-9 (10.5) | SF-1 | PGMEA/DAA/CyHO |
| | | | (100) | (5.0) | Q-B (5.1) | (3.0) | (2,100/600/300) |

TABLE 4

| | | | | Photoacid | Acid diffusion | | |
|---|---|---|---|---|---|---|---|
| | | Resist | Polymer | generator | inhibitor | Surfactant | Solvent |
| | | composition | (pbw) | (pbw) | (pbw) | (pbw) | (pbw) |
| Comparative | 1-1 | CR-1 | P-1 | PAG-1 | Q-A | SF-1 | PGMEA/GBL |
| Example | | | (100) | (8.0) | (2.9) | (3.0) | (1,920/480) |
| | 1-2 | CR-2 | P-1 | PAG-1 | Q-B | SF-1 | PGMEA/GBL |
| | | | (100) | (8.0) | (5.0) | (3.0) | (1,920/480) |
| | 1-3 | CR-3 | P-1 | PAG-1 | Q-C | SF-1 | PGMEA/GBL |
| | | | (100) | (8.0) | (5.0) | (3.0) | (1,920/480) |
| | 1-4 | CR-4 | P-1 | PAG-1 | Q-D | SF-1 | PGMEA/GBL |
| | | | (100) | (8.0) | (5.0) | (3.0) | (1,920/480) |
| | 1-5 | CR-5 | P-1 | PAG-1 | Q-E | SF-1 | PGMEA/GBL |
| | | | (100) | (8.0) | (5.0) | (3.0) | (1,920/480) |
| | 1-6 | CR-6 | P-1 | PAG-1 | Q-F | SF-1 | PGMEA/GBL |
| | | | (100) | (8.0) | (5.0) | (3.0) | (1,920/480) |
| | 1-7 | CR-7 | P-1 | PAG-1 | Q-G | SF-1 | PGMEA/GBL |
| | | | (100) | (8.0) | (5.0) | (3.0) | (1,920/480) |
| | 1-8 | CR-8 | P-1 | PAG-1 | Q-H | SF-1 | PGMEA/GBL |
| | | | (100) | (8.0) | (5.0) | (3.0) | (1,920/480) |
| | 1-9 | CR-9 | P-1 | PAG-1 | Q-I | SF-1 | PGMEA/GBL |
| | | | (100) | (8.0) | (5.0) | (3.0) | (1,920/480) |
| | 1-10 | CR-10 | P-1 | PAG-1 | Q-J | SF-1 | PGMEA/GBL |
| | | | (100) | (8.0) | (5.0) | (3.0) | (1,920/480) |
| | 1-11 | CR-11 | P-1 | PAG-1 | Q-K | SF-1 | PGMEA/GBL |
| | | | (100) | (8.0) | (5.0) | (3.0) | (1,920/480) |
| | 1-12 | CR-12 | P-1 | PAG-1 | Q-L | SF-1 | PGMEA/GBL |
| | | | (100) | (8.0) | (5.0) | (3.0) | (1,920/480) |
| | 1-13 | CR-13 | P-1 | PAG-1 | Q-N | SF-1 | PGMEA/GBL |
| | | | (100) | (8.0) | (5.0) | (3.0) | (1,920/480) |
| | 1-14 | CR-14 | P-2 | PAG-2 | Q-A | SF-1 | PGMEA/DAA |
| | | | (100) | (20.0) | (6.0) | (3.0) | (2,100/900) |
| | 1-15 | CR-15 | P-2 | PAG-2 | Q-B | SF-1 | PGMEA/DAA |
| | | | (100) | (20.0) | (10.0) | (3.0) | (2,100/900) |
| | 1-16 | CR-16 | P-2 | PAG-2 | Q-C | SF-1 | PGMEA/DAA |
| | | | (100) | (20.0) | (10.0) | (3.0) | (2,100/900) |
| | 1-17 | CR-17 | P-2 | PAG-2 | Q-D | SF-1 | PGMEA/DAA |
| | | | (100) | (20.0) | (10.0) | (3.0) | (2,100/900) |
| | 1-18 | CR-18 | P-2 | PAG-2 | Q-E | SF-1 | PGMEA/DAA |
| | | | (100) | (20.0) | (10.0) | (3.0) | (2,100/900) |
| | 1-19 | CR-19 | P-2 | PAG-2 | Q-F | SF-1 | PGMEA/DAA |
| | | | (100) | (20.0) | (10.0) | (3.0) | (2,100/900) |
| | 1-20 | CR-20 | P-2 | PAG-2 | Q-G | SF-1 | PGMEA/DAA |
| | | | (100) | (20.0) | (10.0) | (3.0) | (2,100/900) |
| | 1-21 | CR-21 | P-2 | PAG-2 | Q-H | SF-1 | PGMEA/DAA |
| | | | (100) | (20.0) | (10.0) | (3.0) | (2,100/900) |
| | 1-22 | CR-22 | P-2 | PAG-2 | Q-I | SF-1 | PGMEA/DAA |
| | | | (100) | (20.0) | (10.0) | (3.0) | (2,100/900) |
| | 1-23 | CR-23 | P-2 | PAG-2 | Q-J | SF-1 | PGMEA/DAA |
| | | | (100) | (20.0) | (10.0) | (3.0) | (2,100/900) |
| | 1-24 | CR-24 | P-2 | PAG-2 | Q-K | SF-1 | PGMEA/DAA |
| | | | (100) | (20.0) | (10.0) | (3.0) | (2,100/900) |
| | 1-25 | CR-25 | P-2 | PAG-2 | Q-L | SF-1 | PGMEA/DAA |
| | | | (100) | (20.0) | (10.0) | (3.0) | (2,100/900) |

TABLE 5

| | | | | Photoacid | Acid diffusion | | |
|---|---|---|---|---|---|---|---|
| | | Resist | Polymer | generator | inhibitor | Surfactant | Solvent |
| | | composition | (pbw) | (pbw) | (pbw) | (pbw) | (pbw) |
| Comparative | 1-26 | CR-26 | P-2 | PAG-2 | Q-M | SF-1 | PGMEA/DAA |
| Example | | | (100) | (20.0) | (10.0) | (3.0) | (2,100/900) |
| | 1-27 | CR-27 | P-2 | PAG-2 | Q-N | SF-1 | PGMEA/DAA |
| | | | (100) | (20.0) | (10.0) | (3.0) | (2,100/900) |
| | 1-28 | CR-28 | P-2 | PAG-2 | Q-O | SF-1 | PGMEA/DAA |
| | | | (100) | (20.0) | (10.0) | (3.0) | (2,100/900) |
| | 1-29 | CR-29 | P-4 | — | Q-C | SF-1 | PGMEA/DAA |
| | | | (100) | | (10.0) | (3.0) | (2,100/900) |
| | 1-30 | CR-30 | P-4 | — | Q-F | SF-1 | PGMEA/DAA |
| | | | (100) | | (10.0) | (3.0) | (2,100/900) |
| | 1-31 | CR-31 | P-4 | — | Q-H | SF-1 | PGMEA/DAA |
| | | | (100) | | (10.0) | (3.0) | (2,100/900) |
| | 1-32 | CR-32 | P-4 | — | Q-I | SF-1 | PGMEA/DAA |
| | | | (100) | | (10.0) | (3.0) | (2,100/900) |
| | 1-33 | CR-33 | P-4 | — | Q-J | SF-1 | PGMEA/DAA |
| | | | (100) | | (10.0) | (3.0) | (2,100/900) |
| | 1-34 | CR-34 | P-4 | — | Q-K | SF-1 | PGMEA/DAA |
| | | | (100) | | (10.0) | (3.0) | (2,100/900) |
| | 1-35 | CR-35 | P-4 | — | Q-L | SF-1 | PGMEA/DAA |
| | | | (100) | | (10.0) | (3.0) | (2,100/900) |
| | 1-36 | CR-36 | P-4 | — | Q-M | SF-1 | PGMEA/DAA |
| | | | (100) | | (10.0) | (3.0) | (2,100/900) |
| | 1-37 | CR-37 | P-4 | — | Q-N | SF-1 | PGMEA/DAA |
| | | | (100) | | (10.0) | (3.0) | (2,100/900) |

### Examples 3-1 to 3-16 and Comparative Examples 2-1 to 2-13

ArF Lithography Patterning Test

On a silicon substrate, an antireflective coating solution (ARC-29A by Nissan Chemical Corp.) was coated and baked at 180° C. for 60 seconds to form an ARC of 100 nm thick. On the ARC, each of the resist compositions (R-1 to R-16, CR-1 to CR-13) was spin coated and baked on a hotplate at 100° C. for 60 seconds to form a resist film of 90 nm thick.

Using an ArF excimer laser scanner (NSR-S610C by Nikon Corp., NA 1.30, σ 0.94/0.74, dipole 35 deg. illumination, 6% halftone phase shift mask), the resist film was exposed by the immersion lithography. Water was used as the immersion liquid. After to exposure, the resist film was baked (PEB) at 90° C. for 60 seconds and developed in 2.38 wt % TMAH aqueous solution for 60 seconds to form a line-and-space (LS) pattern.

The LS pattern as developed was observed under CD-SEM (CG-5000 by Hitachi High-Technologies Corp.) and evaluated for sensitivity and LWR by the following methods. The results are shown in Table 6.

### Evaluation of Sensitivity

The optimum dose (Eop) is a dose (mJ/cm²) which provides a LS pattern having a line width of 40 nm at a pitch of 80 nm and reported as sensitivity. A smaller value indicates a higher sensitivity.

### Evaluation of LWR

On the L/S pattern formed by exposure in the optimum dose Eop, the line width was measured at longitudinally spaced apart 10 points, from which a 3-fold value (3σ) of standard deviation (σ) was determined and reported as LWR. A smaller value of 3σ indicates a pattern having a lower roughness and more uniform line width. A pattern with a LWR value of 2.5 nm or less is rated good while a pattern with a LWR value in excess of 2.5 nm is rated NG.

TABLE 6

| | | Resist composition | Eop (mJ/cm²) | LWR (nm) |
|---|---|---|---|---|
| Example | 3-1 | R-1 | 32 | Good (2.3) |
| | 3-2 | R-2 | 36 | Good (2.2) |
| | 3-3 | R-3 | 37 | Good (2.1) |
| | 3-4 | R-4 | 36 | Good (2.2) |
| | 3-5 | R-5 | 32 | Good (2.1) |
| | 3-6 | R-6 | 33 | Good (2.3) |
| | 3-7 | R-7 | 33 | Good (2.3) |
| | 3-8 | R-8 | 33 | Good (2.5) |
| | 3-9 | R-9 | 36 | Good (2.1) |
| | 3-10 | R-10 | 35 | Good (2.2) |
| | 3-11 | R-11 | 35 | Good (2.1) |
| | 3-12 | R-12 | 35 | Good (2.4) |
| | 3-13 | R-13 | 36 | Good (2.5) |
| | 3-14 | R-14 | 37 | Good (2.4) |
| | 3-15 | R-15 | 35 | Good (2.1) |
| | 3-16 | R-16 | 35 | Good (2.2) |
| Comparative | 2-1 | CR-1 | 46 | NG (3.3) |
| Example | 2-2 | CR-2 | 42 | NG (2.7) |
| | 2-3 | CR-3 | 35 | NG (3.2) |
| | 2-4 | CR-4 | 36 | NG (2.8) |
| | 2-5 | CR-5 | 37 | NG (2.7) |
| | 2-6 | CR-6 | 39 | NG (2.8) |
| | 2-7 | CR-7 | 34 | NG (3.1) |
| | 2-8 | CR-8 | 34 | NG (3.1) |
| | 2-9 | CR-9 | 38 | NG (2.7) |
| | 2-10 | CR-10 | 37 | NG (2.7) |
| | 2-11 | CR-11 | 35 | NG (2.9) |
| | 2-12 | CR-12 | 38 | NG (2.6) |
| | 2-13 | CR-13 | 35 | NG (2.8) |

As is evident from Table 6, the chemically amplified resist compositions containing onium salt compounds within the scope of the invention exhibit a good balance of sensitivity and LWR. The resist compositions are useful as the ArF immersion lithography material.

### Examples 4-1 to 4-63 and Comparative Examples 3-1 to 3-24

EUV Lithography Test

Each of the resist compositions (R-17 to R-79, CR-14 to CR-37) was spin coated on a silicon substrate having a 20-nm coating of silicon-containing spin-on hard mask SHB-A940 (silicon content 43 wt %, Shin-Etsu Chemical Co., Ltd.) and prebaked on a hotplate at 105° C. for 60 seconds to form a resist film of 50 nm thick. Using an EUV scanner NXE3300 (ASML, NA 0.33, σ 0.9/0.6, quadrupole illumination), the resist film was exposed to EUV through a mask bearing a hole pattern having a pitch 46 nm+20% bias (on-wafer size). The resist film was baked (PEB) on a hotplate at 85° C. for 60 seconds and developed in a to 2.38 wt % TMAH aqueous solution for 30 seconds to form a hole pattern having a size of 23 nm.

The hole pattern as developed was observed under CD-SEM (CG-5000 by Hitachi High-Technologies Corp.) and evaluated for sensitivity and CDU by the following methods. The results are shown in Tables 7 to 9.

### Evaluation of Sensitivity

The optimum dose (Eop) is a dose (mJ/cm²) which provides a hole pattern having a hole size of 23 nm and reported as sensitivity. A smaller value indicates a higher sensitivity.

### Evaluation of CDU

For the hole pattern at the optimum dose (Eop), the size of 50 holes within the same dose shot was measured, from which a 3-fold value (3σ) of standard deviation (σ) was computed and reported as CDU. A smaller value of CDU indicates better dimensional uniformity of hole pattern. The sample was rated good for a CDU value of up to 3.0 nm and NG for a CDU value in excess of 3.0 nm.

TABLE 7

| | | Resist composition | Eop (mJ/cm²) | CDU (nm) |
|---|---|---|---|---|
| Example | 4-1 | R-17 | 30 | Good (2.7) |
| | 4-2 | R-18 | 30 | Good (2.7) |
| | 4-3 | R-19 | 33 | Good (2.8) |
| | 4-4 | R-20 | 32 | Good (2.8) |
| | 4-5 | R-21 | 31 | Good (2.8) |
| | 4-6 | R-22 | 34 | Good (2.7) |
| | 4-7 | R-23 | 28 | Good (2.9) |
| | 4-8 | R-24 | 33 | Good (2.7) |
| | 4-9 | R-25 | 30 | Good (2.7) |
| | 4-10 | R-26 | 31 | Good (2.8) |
| | 4-11 | R-27 | 32 | Good (2.9) |
| | 4-12 | R-28 | 31 | Good (2.8) |
| | 4-13 | R-29 | 31 | Good (2.8) |
| | 4-14 | R-30 | 30 | Good (2.7) |
| | 4-15 | R-31 | 31 | Good (2.7) |
| | 4-16 | R-32 | 33 | Good (2.6) |
| | 4-17 | R-33 | 31 | Good (2.8) |
| | 4-18 | R-34 | 34 | Good (2.6) |
| | 4-19 | R-35 | 32 | Good (2.9) |
| | 4-20 | R-36 | 31 | Good (2.7) |
| | 4-21 | R-37 | 32 | Good (2.8) |
| | 4-22 | R-38 | 34 | Good (3.0) |
| | 4-23 | R-39 | 34 | Good (2.8) |
| | 4-24 | R-40 | 31 | Good (2.8) |
| | 4-25 | R-41 | 34 | Good (2.6) |
| | 4-26 | R-42 | 30 | Good (2.7) |
| | 4-27 | R-43 | 31 | Good (2.9) |
| | 4-28 | R-44 | 32 | Good (3.0) |
| | 4-29 | R-45 | 30 | Good (2.6) |
| | 4-30 | R-46 | 29 | Good (2.5) |
| | 4-31 | R-47 | 31 | Good (2.8) |
| | 4-32 | R-48 | 30 | Good (2.6) |

TABLE 8

| | | Resist composition | Eop (mJ/cm²) | CDU (nm) |
|---|---|---|---|---|
| Example | 4-33 | R-49 | 32 | Good (2.6) |
| | 4-34 | R-50 | 30 | Good (2.5) |
| | 4-35 | R-51 | 30 | Good (2.7) |
| | 4-36 | R-52 | 30 | Good (2.7) |
| | 4-37 | R-53 | 29 | Good (2.6) |
| | 4-38 | R-54 | 32 | Good (2.7) |
| | 4-39 | R-55 | 27 | Good (2.6) |
| | 4-40 | R-56 | 27 | Good (2.5) |
| | 4-41 | R-57 | 28 | Good (2.7) |
| | 4-42 | R-58 | 27 | Good (2.6) |
| | 4-43 | R-59 | 24 | Good (2.8) |
| | 4-44 | R-60 | 26 | Good (2.6) |
| | 4-45 | R-61 | 26 | Good (2.5) |
| | 4-46 | R-62 | 26 | Good (2.4) |
| | 4-47 | R-63 | 27 | Good (2.7) |
| | 4-48 | R-64 | 26 | Good (2.6) |
| | 4-49 | R-65 | 27 | Good (2.6) |
| | 4-50 | R-66 | 25 | Good (2.5) |
| | 4-51 | R-67 | 25 | Good (2.5) |
| | 4-52 | R-68 | 27 | Good (2.7) |
| | 4-53 | R-69 | 29 | Good (2.6) |
| | 4-54 | R-70 | 27 | Good (2.8) |
| | 4-55 | R-71 | 29 | Good (2.7) |
| | 4-56 | R-72 | 28 | Good (2.8) |
| | 4-57 | R-73 | 26 | Good (2.6) |
| | 4-58 | R-74 | 28 | Good (2.9) |
| | 4-59 | R-75 | 23 | Good (2.6) |
| | 4-60 | R-76 | 23 | Good (2.6) |
| | 4-61 | R-77 | 24 | Good (2.5) |
| | 4-62 | R-78 | 25 | Good (2.4) |
| | 4-63 | R-79 | 24 | Good (2.3) |

TABLE 9

| | | Resist composition | Eop (mJ/cm²) | CDU (nm) |
|---|---|---|---|---|
| Comparative | 3-1 | RC-14 | 43 | NG (3.6) |
| Example | 3-2 | RC-15 | 34 | NG (3.1) |
| | 3-3 | RC-16 | 33 | NG (3.5) |
| | 3-4 | RC-17 | 33 | NG (3.3) |
| | 3-5 | RC-18 | 32 | NG (3.3) |
| | 3-6 | RC-19 | 37 | NG (3.2) |
| | 3-7 | RC-20 | 38 | NG (3.4) |
| | 3-8 | RC-21 | 37 | NG (3.4) |
| | 3-9 | RC-22 | 32 | NG (3.3) |
| | 3-10 | RC-23 | 32 | NG (3.3) |
| | 3-11 | RC-24 | 34 | NG (3.3) |
| | 3-12 | RC-25 | 32 | NG (3.1) |
| | 3-13 | RC-26 | 33 | NG (3.3) |
| | 3-14 | RC-27 | 34 | NG (3.3) |
| | 3-15 | RC-28 | 25 | NG (3.6) |
| | 3-16 | RC-29 | 29 | NG (3.3) |
| | 3-17 | RC-30 | 32 | NG (3.2) |
| | 3-18 | RC-31 | 30 | NG (3.3) |
| | 3-19 | RC-32 | 32 | NG (3.4) |
| | 3-20 | RC-33 | 31 | NG (3.3) |
| | 3-21 | RC-34 | 31 | NG (3.4) |
| | 3-22 | RC-35 | 30 | NG (3.1) |
| | 3-23 | RC-36 | 33 | NG (3.2) |
| | 3-24 | RC-37 | 30 | NG (3.3) |

As is evident from Tables 7 to 9, the chemically amplified resist compositions containing onium salt compounds within the scope of the invention exhibit high sensitivity and satisfactory values of CDU. The resist compositions are useful as the EUV lithography material.

Japanese Patent Application No. 2019-224690 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. An onium salt compound having the formula (1): 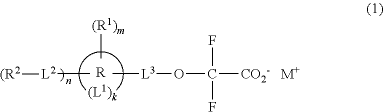
wherein m, n and k are each independently 0 or a positive integer, m+n+k is at least 1,
R¹ is halogen, trifluoromethyl or trifluoromethoxy,
R² is hydrogen or a C₁-C₁₅ hydrocarbyl group which may contain a heteroatom,
L¹ is —C(═O)—, —C(═O)—O—, —S(═O)—, —S(═O)₂— or —S(═O)₂—O—,
L² is *—C(═O)—, *—C(═O)—O—, *—S(═O)—, *—S(═O)₂— or *—S(═O)₂—O—, * designates a valence bond to the ring R,
L³ is a single bond or C₁-C₁₅ hydrocarbylene group, some hydrogen in the hydrocarbylene group may be substituted by a heteroatom-containing moiety, —CH₂— in the hydrocarbylene group may be replaced by —O—, —C(═O)—, —S—, —S(═O)—, —S(═O)₂— or —N(R^{N})—, with the proviso that when L³ is a hydrocarbylene group, the carbon atom bonding to —OCF₂CO₂⁻ in the formula does not bond to a heteroatom other than the oxygen atom in the formula, R^{N} is hydrogen or a C₁-C₁₀ hydrocarbyl group, some hydrogen in the hydrocarbyl group R^{N} may be substituted by a heteroatom-containing moiety, —CH₂— in the hydrocarbyl group R^{N} may be replaced by —O—, —C(═O)—, or —S(═O)₂—,
the ring R is a (m+n+1)-valent cyclic hydrocarbon group when k=0, and a (m+n+1)-valent cyclic hydrocarbon group containing k number of L¹ when k is a positive integer, some hydrogen in the cyclic hydrocarbon group may be substituted by a heteroatom-containing moiety, —CH₂— in the cyclic hydrocarbon group may be replaced by —O— or —S—, and
M⁺ is a sulfonium or iodonium cation.

2. The onium salt compound of claim 1 wherein L³ is a single bond.

3. The onium salt compound of claim 1 wherein the ring R is an aromatic hydrocarbon group.

4. The onium salt compound of claim 1 wherein m is an integer of at least 1.

5. The onium salt compound of claim 1, having the formula (2): 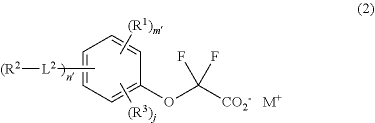
wherein R¹, R², L², and M⁺ are as defined above,
m′ is an integer of 0 to 5, n′ is an integer of 0 to 5, and j is an integer of 0 to 4, 1≤m′+n′≤5 and 1≤m′+n′+j≤5,
R³ is hydrogen, hydroxyl, carboxyl or a C₁-C₁₅ hydrocarbyl group, some hydrogen in the hydrocarbyl group may be substituted by a heteroatom-containing moiety, —CH₂— in the hydrocarbyl group may be replaced by —O— or —C(═O)—, and when j is an integer of 2 to 4, a plurality of R³ may be the same or different, or two R³ may bond together to form a ring with the carbon atoms to which they are attached.

6. The onium salt compound of claim 5 wherein m′ is an integer of at least 1.

7. The onium salt compound of claim 1 wherein R¹ is iodine.

8. The onium salt compound of claim 1 wherein M⁺ is a cation having any one of the following formulae (M-1) to (M-4): 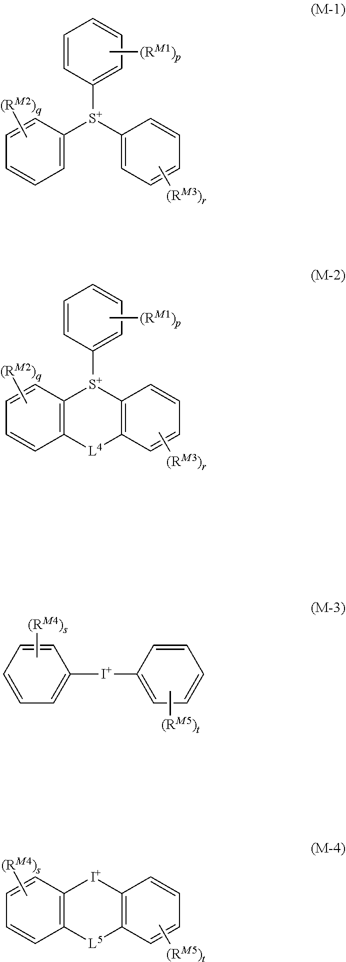
wherein R^{M1}, R^{M2}, R^{M3}, R^{M4}, and R^{M5} are each independently halogen, hydroxyl, or a C₁-C₁₅ hydrocarbyl group, some hydrogen in the hydrocarbyl group may be substituted by a heteroatom-containing moiety, —CH₂— in the hydrocarbyl group may be replaced by —O—, —C(═O)—, —S—, —S(═O)—, —S(═O)₂— or —N(R^{N})—,
L⁴ and L⁵ are each independently a single bond, —CH₂—, —O—, —C(═O)—, —S—, —S(═O)—, —S(═O)₂— or —N(R^{N})—,
R^{N} is hydrogen or a C₁-C₁₀ hydrocarbyl group, some hydrogen in the hydrocarbyl group may be substituted by a heteroatom-containing moiety, —CH₂— in the hydrocarbyl group may be replaced by —O—, —C(═O)— or —S(═O)₂—,
p, q, r, s and t are each independently an integer of 0 to 5,
when p is 2 or more, a plurality of R^{M1} may be the same or different, and two R^{M1} may bond together to form a ring with the carbon atoms on the benzene ring to which they are attached, when q is 2 or more, a plurality of R^{M2} may be the same or different, and two R^{M2} may bond together to form a ring with the carbon atoms on the benzene ring to which they are attached, when r is 2 or more, a plurality of R^{M3} may be the same or different, and two R^{M3} may bond together to form a ring with the carbon atoms on the benzene ring to which they are attached, when s is 2 or more, a plurality of R^{M4} may be the same or different, and two R^{M4} may bond together to form a ring with the carbon atoms on the benzene ring to which they are attached, when t is 2 or more, a plurality of R^{M5} may be the same or different, and two R^{M5} may bond together to form a ring with the carbon atoms on the benzene ring to which they are attached.

9. The onium salt compound of claim 8, having the following formula (3) or (4): 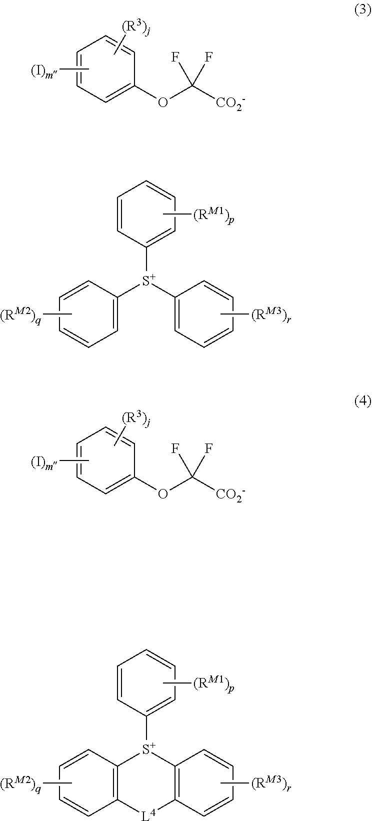
wherein R^{M1}, R^{M2}, R^{M3}, R³, L⁴, p, q, and r are as defined above, m″ is an integer of 1 to 5, and j is an integer of 0 to 4, and m″+j is from 1 to 5.

10. An acid diffusion inhibitor comprising the onium salt compound of claim 1.

11. A chemically amplified resist composition comprising (A) a base polymer adapted to change its solubility in a developer under the action of an acid, (B) a photoacid generator, (C) an acid diffusion inhibitor comprising the onium salt compound of claim 1, and (D) an organic solvent.

12. A chemically amplified resist composition comprising (A′) a base polymer adapted to change its solubility in a developer under the action of an acid, the base polymer comprising recurring units having a function of generating an acid upon exposure to light, (C) an acid diffusion inhibitor comprising the onium salt compound of claim 1, and (D) an organic solvent.

13. The resist composition of claim 11 wherein the base polymer comprises recurring units having the formula (a) or recurring units having the formula (b): 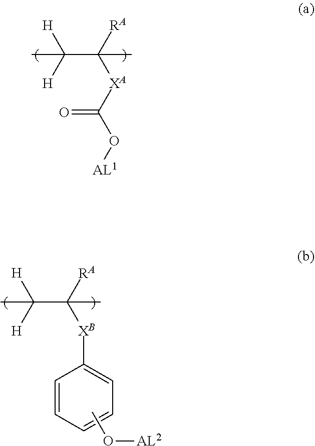
wherein R^{A} is hydrogen or methyl, X^{A} is a single bond, phenylene group, naphthylene group or (backbone)-C(═O)—O—X^{A1}—, X^{A1} is a C₁-C₁₅ hydrocarbylene group which may contain a hydroxyl moiety, ether bond, ester bond or lactone ring, X^{B} is a single bond or ester bond, AL¹ and AL² are each independently an acid labile group.

14. The resist composition of claim 13 wherein the acid labile group has the formula (L1): 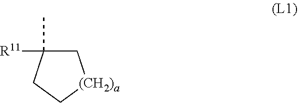
wherein R¹¹ is a C₁-C₇ hydrocarbyl group in which —CH₂— may be replaced by —O—, a is 1 or 2, and the broken line designates a valence bond.

15. The resist composition of claim 11 wherein the base polymer comprises recurring units having the formula (c): 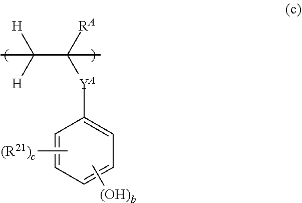
wherein R^{A} is hydrogen or methyl, Y^{A} is a single bond or ester bond, R²¹ is fluorine, iodine or a C₁-C₁₀ hydrocarbyl group in which —CH₂— may be replaced by —O— or —C(═O)—, b is an integer of 1 to 5, c is an integer of 0 to 4, and b+c is from 1 to 5.

16. The resist composition of claim 12 wherein the recurring units having a function of generating an acid upon exposure to light are units of at least one type selected from the formulae (d1) to (d4): 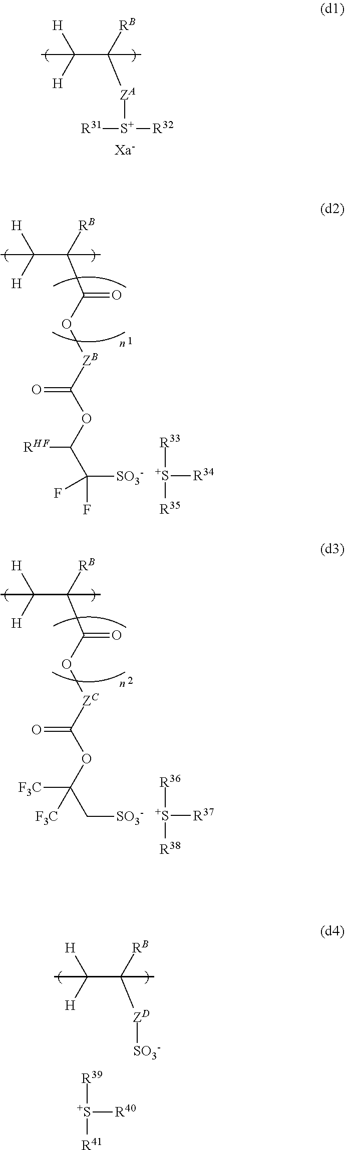
wherein R^{B} is hydrogen, fluorine, methyl or trifluoromethyl,
Z^{A} is a single bond, phenylene group, —O—Z^{A1}—, —C(═O)—O—Z^{A1}— or —C(═O)—NH—Z^{A1}—, Z^{A1} is a C₁-C₂₀ hydrocarbylene group which may contain a heteroatom,
Z^{B} and Z^{C} are each independently a single bond or a C₁-C₂₀ hydrocarbylene group which may contain a heteroatom,
Z^{D} is a single bond, methylene, ethylene, phenylene, fluorinated phenylene, —O—Z^{D1}—, —C(═O)—O—Z^{D1}— or —C(═O)—NH—Z^{D1}—, Z^{D1} is an optionally substituted phenylene group,
R³¹ to R⁴¹ are each independently a C₁-C₂₀ hydrocarbyl group which may contain a heteroatom, any two of Z^{A}, R³¹ and R³² may bond together to form a ring with the sulfur atom to which they are attached, any two of R³³, R³⁴ and R³⁵, any two of R³⁶, R³⁷ and R³⁸, and any two of R³⁹, R⁴⁰ and R⁴¹ may bond together to form a ring with the sulfur atom to which they are attached,
R^{HF} is hydrogen or trifluoromethyl,
n¹ is 0 or 1, n¹ is 0 when Z^{B} is a single bond, n² is 0 or 1, n² is 0 when Z^{C} is a single bond, and
Xa⁻ is a non-nucleophilic counter ion.

17. A pattern forming process comprising the steps of applying the chemically amplified resist composition of claim 11 to form a resist film on a substrate, exposing a selected region of the resist film to KrF excimer laser, ArF excimer laser, EB or EUV, and developing the exposed resist film in a developer.

18. The pattern forming process of claim 17 wherein the developing step uses an alkaline aqueous solution as the developer, thereby forming a positive pattern in which an exposed region of the resist film is dissolved away and an unexposed region of the resist film is not dissolved.

19. The pattern forming process of claim 17 wherein the developing step uses an organic solvent as the developer, thereby forming a negative pattern in which an unexposed region of the resist film is dissolved away and an exposed region of the resist film is not dissolved.

20. The pattern forming process of claim 19 wherein the organic solvent is at least one solvent selected from the group consisting of 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate.
